(19) United States
(12) Patent Application Publication
(10) Pub. No.: US 2009/0298834 A1
(43) Pub. Date: Dec. 3, 2009

(54) 4-(AMINOMETHYL)CYCLOHEXANAMINE DERIVATIVES AS CALCIUM CHANNEL BLOCKERS

(76) Inventors: Hassan PAJOUHESH, West Vancouver (CA); Ramesh Kaul, Burnaby (CA); Mike Grimwood, North Vancouver (CA); Jason Tan, Vancouver (CA); Yuanxi Zhou, Richmond (CA)

Correspondence Address: MORRISON & FOERSTER LLP, 12531 HIGH BLUFF DRIVE, SUITE 100, SAN DIEGO, CA, 92130-2040, US

(21) Appl. No.: 12/420,785

(22) Filed: Apr. 8, 2009

Related U.S. Application Data
(60) Provisional application No. 61/058,183, filed on Jun. 2, 2008.
(60) Provisional application No. 61/058,185, filed on Jun. 2, 2008.

(51) Int. Cl. A61K 31/5375; C07C 233/74; A61K 31/166; A61K 31/27; C07C 271/24; C07D 295/108

(52) U.S. Cl. 514/237.5; 564/186; 514/617; 514/490; 560/27; 544/165

(57) ABSTRACT

Methods and compounds effective in ameliorating conditions characterized by unwanted calcium channel activity, particularly unwanted T-type calcium channel activity are disclosed. Specifically, a series of compounds containing 4-(aminomethyl)cyclohexanamine derivatives as shown in formula (1).

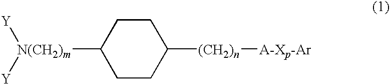

### CROSS-REFERENCE TO RELATED APPLICATIONS

This application claims benefit of Provisional Application Ser. Nos. 61/058,183, filed 2 Jun. 2008, and 61/058,185, filed 2 Jun. 2008. The contents of these documents are incorporated herein by reference.

### TECHNICAL FIELD

The invention relates to compounds useful in treating conditions associated with calcium channel function, and particularly conditions associated with T-type calcium channel activity. More specifically, the invention concerns compounds containing substituted 4-(aminomethyl)cyclohexanamine derivatives that are useful in treatment of conditions such as cardiovascular disease, epilepsy and pain.

### BACKGROUND ART

The entry of calcium into cells through voltage-gated calcium channels mediates a wide variety of cellular and physiological responses, including excitation-contraction coupling, hormone secretion and gene expression (Miller, R. J., Science (1987) 235:46-52; Augustine, G. J. et al., Annu Rev Neurosci (1987) 10: 633-693). In neurons, calcium channels directly affect membrane potential and contribute to electrical properties such as excitability, repetitive firing patterns and pacemaker activity. Calcium entry further affects neuronal functions by directly regulating calcium-dependent ion channels and modulating the activity of calcium-dependent enzymes such as protein kinase C and calmodulin-dependent protein kinase II. An increase in calcium concentration at the presynaptic nerve terminal triggers the release of neurotransmitter, which also affects neurite outgrowth and growth cone migration in developing neurons.

Calcium channels mediate a variety of normal physiological functions, and are also implicated in a number of human disorders. Examples of calcium-mediated human disorders include but are not limited to congenital migraine, cerebellar ataxia, angina, epilepsy, hypertension, ischemia, and some arrhythmias. The clinical treatment of some of these disorders has been aided by the development of therapeutic calcium channel antagonists (e.g., dihydropyridines, phenylalkyl amines, and benzothiazapines all target L-type calcium channels) (Janis, R. J. & Triggle, D. J., In Calcium Channels: Their Properties, Functions, Regulation nad Clinical Relevance (1991) CRC Press, London).

Native calcium channels have been classified by their electrophysiological and pharmacological properties into T-, L-, N-, P/ Q- and R-types (reviewed in Catterall, W., Annu Rev Cell Dev Biol (2000) 16: 521-555; Huguenard, J. R., Annu Rev Physiol (1996) 58: 329-348). T-type (or low voltage-activated) channels describe a broad class of molecules that transiently activate at negative potentials and are highly sensitive to changes in resting potential.

The L-, N- and P/Q-type channels activate at more positive potentials (high voltage-activated) and display diverse kinetics and voltage-dependent properties (Catterall (2000); Huguenard (1996)). T-type channels can be distinguished by having a more negative range of activation and inactivation, rapid inactivation, slow deactivation, and smaller single-channel conductances. There are three subtypes of T-type calcium channels that have been molecularly, pharmacologically, and elecrophysiologically identified: these subtypes have been termed (X1G, α_{1H}, and α_{1I} (alternately called Cav 3.1, Cav 3.2 and Cav 3.3 respectively).

T-type calcium channels are involved in various medical conditions. In mice lacking the gene expressing the α_{1G} subunit, resistance to absence seizures was observed (Kim, C. et al., Mol Cell Neurosci (2001) 18(2): 235-245). Other studies have also implicated the α_{1H} subunit in the development of epilepsy (Su, H. et al., J Neurosci (2002) 22: 3645-3655). There is strong evidence that some existing anticonvulsant drugs, such as ethosuximide, function through the blockade of T-type channels (Gomora, J. C., et al., Mol Pharmacol (2001) 60: 1121-1132).

Low voltage-activated calcium channels are highly expressed in tissues of the cardiovascular system. Mibefradil, a calcium channel blocker 10-30 fold selective for T-type over L-type channels, was approved for use in hypertension and angina. It was withdrawn from the market shortly after launch due to interactions with other drugs (Heady, T. N., et al., Jpn J Pharmacol. (2001) 85:339-350).

Growing evidence suggests T-type calcium channels are also involved in pain (see for example: US Patent Application No. 2003/086980; PCT Patent Application Nos. WO 03/007953 and WO 04/000311). Both mibefradil and ethosuximide have shown anti-hyperalgesic activity in the spinal nerve ligation model of neuropathic pain in rats (Dogrul, A., et al., Pain (2003) 105:159-168). In addition to cardiovascular disease, epilepsy (see also US Patent Application No. 2006/025397), and chronic and acute pain, T-type calcium channels have been implicated in diabetes (US Patent Application No. 2003/125269), certain types of cancer such as prostate cancer (PCT Patent Application Nos. WO 05/086971 and WO 05/77082), sleep disorders (US Patent Application No. 2006/003985), Parkinson's disease (US Patent Application No. 2003/087799); psychosis such as schizophrenia (US Patent Application No. 2003/087799), overactive bladder (Sui, G.-P., et al., British Journal of Urology International (2007) 99(2): 436-441; see also US 2004/197825), renal disease (Hayashi, K., et al., Journal of Pharmacological Sciences (2005) 99: 221-227), neuroprotection and male birth control.

All patents, patent applications and publications are herein incorporated by reference in their entirety.

### DISCLOSURE OF THE INVENTION

The invention relates to compounds useful in treating conditions modulated by calcium channel activity and in particular conditions mediated by T-type channel activity. The compounds of the invention are N-piperidinyl acetamide derivatives with structural features that enhance the calcium channel blocking activity of the compounds. Thus, in one aspect, the invention is directed to a method of treating conditions mediated by calcium channel activity by administering to patients in need of such treatment at least one compound of formula (1):

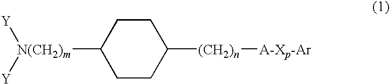

or a pharmaceutically acceptable salt or conjugate thereof, wherein

A is C(O)NH or NHC(O);

X is an optionally substituted alkylene (1-4C), heteroalkylene (2-4C), alkenylene (2-4C), or heteroakenylene (2-4C);

m, n and p are independently 0 or 1;

Ar is an optionally substituted aryl (6-10C) or heteroaryl (5-12 ring members);

each Y is independently H, SR′, SOR′, SO₂R′, wherein each R′ is independently H or an optionally substituted group selected from alkyl (1-6C), alkenyl (2-6C), alkynyl (2-6C), heteroalkyl (2-6C), heteroalkenyl (2-6), heteroalkynyl (2-6C); or each Y is an optionally substituted group selected from alkyl (1-10C), alkenyl (2-10C), alkynyl (2-10C), heteroalkyl (2-10C), heteroalkenyl (2-10C), heteroalkynyl (2-10C), aryl (6-12C)-alkyl (1-6C) or heteroaryl (5-12 ring members)-alkyl (1-6C); or two Y may together form an optionally substituted heterocyclic ring (4-6 ring members);

wherein the optional substituents on X, Y and Ar may be one or more halo, CN, NO₂, CF₃, OCF₃, COOR′, CONR′₂, OR′, SR′, SOR′, SO₂R′, NR′₂, NR′(CO)R′, NR′C(O)OR′, NR′C(O)NR′₂, NR′SO₂NR′₂, NR′SO₂R′, wherein each R′ is independently H or an optionally substituted group selected from alkyl (1-6C), alkenyl (2-6C), alkynyl (2-6C), heteroalkyl (2-6C) heteroalkenyl (2-6), heteroalkynyl (2-6C); or each substituent is alkyl (1-6C), alkenyl (2-6C), alkynyl (2-6C), heteroalkyl (2-6C), heteroalkenyl (2-6C), heteroalkynyl (2-6C); aryl (6-10C), heteroaryl (5-12 ring members), O-aryl (6-10C), O-heteroaryl (5-12 ring members), aryl (6-12C)-alkyl (1-6C) or heteroaryl (5-12 ring members)-alkyl (1-6C); and wherein optional substituents on X and Y may be additionally selected from ═O, ═NOR′.

The invention is also directed to the use of compounds of formula (1) for the preparation of medicaments for the treatment of conditions requiring modulation of calcium channel activity, and in particular T-type calcium channel activity. In another aspect, the invention is directed to pharmaceutical compositions containing compounds of formula (1) in admixture with a pharmaceutically acceptable excipient with the additional provisos that Ar is not a naphthyl and that the two Y do not together form a pyrrolidin-2-onyl ring. In yet another aspect, the invention is directed to the use of these compositions for treating conditions requiring modulation of calcium channel activity, and particularly T-type calcium channel activity. The invention is also directed to compounds of formula (1) useful to modulate calcium channel activity, particularly T-type channel activity.

### DETAILED DESCRIPTION

As used herein, the term “alkyl,” “alkenyl” and “alkynyl” include straight-chain, branched-chain and cyclic monovalent substituents, as well as combinations of these, containing only C and H when unsubstituted. Examples include methyl, ethyl, isobutyl, cyclohexyl, cyclopentylethyl, 2-propenyl, 3-butynyl, and the like. Typically, the alkyl, alkenyl and alkynyl groups contain 1-10C (alkyl) or 2-10C (alkenyl or alkynyl). In some embodiments, they contain 1-8C, 1-6C, 1-4C, 1-3C or 1-2C (alkyl); or 2-8C, 2-6C, 2-4C or 2-3C (alkenyl or alkynyl). Further, any hydrogen atom on one of these groups can be replaced with a halogen atom, and in particular a fluoro or chloro, and still be within the scope of the definition of alkyl, alkenyl and alkynyl. For example, CF₃ is a 1C alkyl. These groups may be also be substituted by other substituents.

Heteroalkyl, heteroalkenyl and heteroalkynyl are similarly defined and contain at least one carbon atom but also contain one or more O, S or N heteroatoms or combinations thereof within the backbone residue whereby each heteroatom in the heteroalkyl, heteroalkenyl or heteroalkynyl group replaces one carbon atom of the alkyl, alkenyl or alkynyl group to which the heteroform corresponds. In some embodiments, the heteroalkyl, heteroalkenyl and heteroalkynyl groups have C at each terminus to which the group is attached to other groups, and the heteroatom(s) present are not located at a terminal position. As is understood in the art, these heteroforms do not contain more than three contiguous heteroatoms. In some embodiments, the heteroatom is O or N.

The designated number of carbons in heteroforms of alkyl, alkenyl and alkynyl includes the heteroatom count. For example, if heteroalkyl is defined as 1-6C, it will contain 1-6 C, N, O, or S atoms such that the heteroalkyl contains at least one C atom and at least one heteroatom, for example 1-5C and 1N or 1-4C and 2N. Similarly, when heteroalkyl is defined as 1-6C or 1-4C, it would contain 1-5C or 1-3C respectively, i.e., at least one C is replaced by O, N or S. Accordingly, when heteroalkenyl or heteroalkynyl is defined as 2-6C (or 2-4C), it would contain 2-6 or 2-4 C, N, O, or S atoms, since the heteroalkenyl or heteroalkynyl contains at least one carbon atom and at least one heteroatom, e.g. 2-5C and 1N or 2-4C and 2O. Further, heteroalkyl, heteroalkenyl or heteroalkynyl substituents may also contain one or more carbonyl groups. Examples of heteroalkyl, heteroalkenyl and heteroalkynyl groups include CH₂OCH₃, CH₂N(CH₃)₂, CH₂OH, (CH₂)ₙNR₂, OR, COOR, CONR₂, (CH₂)ₙOR, (CH₂)ₙCOR, (CH₂)ₙCOOR, (CH₂)ₙSR, (CH₂)ₙSOR, (CH₂)ₙSO₂R, (CH₂)ₙCONR₂, NRCOR, NRCOOR, OCONR₂, OCOR and the like wherein the group contains at least one C and the size of the substituent is consistent with the definition of alkyl, alkenyl and alkynyl.

“Aromatic” moiety or “aryl” moiety refers to any monocyclic or fused ring bicyclic system which has the characteristics of aromaticity in terms of electron distribution throughout the ring system and includes a monocyclic or fused bicyclic moiety such as phenyl or naphthyl; “heteroaromatic” or “heteroaryl” also refers to such monocyclic or fused bicyclic ring systems containing one or more heteroatoms selected from O, S and N. The inclusion of a heteroatom permits inclusion of 5-membered rings to be considered aromatic as well as 6-membered rings. Thus, typical aromatic/heteroaromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, isoxazolyl, benzoxazolyl, benzoisoxazolyl, imidazolyl and the like. Because tautomers are theoretically possible, phthalimido is also considered aromatic. Typically, the ring systems contain 5-12 ring member atoms or 6-10 ring member atoms. In some embodiments, the aromatic or heteroaromatic moiety is a 6-membered aromatic rings system optionally containing 1-2 nitrogen atoms. More particularly, the moiety is an optionally substituted phenyl, pyridyl, indolyl, pyrimidyl, pyridazinyl, benzothiazolyl or benzimidazolyl, pyrazolyl, imidazolyl, isoxazolyl, thiazolyl, benzothiazolyl, indolyl. Even more particularly, such moiety is phenyl, pyridyl, or pyrimidyl and even more particularly, it is phenyl.

“O-aryl” or “O-heteroaryl” refers to aromatic or heteroaromatic systems which are coupled to another residue through an oxygen atom. A typical example of an O-aryl is phenoxy. Similarly, “arylalkyl” refers to aromatic and heteroaromatic systems which are coupled to another residue through a carbon chain, saturated or unsaturated, typically of 1-8C, 1-6C or more particularly 1-4C or 1-3C when saturated or 2-8C, 2-6C, 2-4C or 2-3C when unsaturated, including the heteroforms thereof. For greater certainty, arylalkyl thus includes an aryl or heteroaryl group as defined above connected to an alkyl, heteroalkyl, alkenyl, heteroalkenyl, alkynyl or heteroalkynyl moiety also as defined above. Typical arylalkyls would be an aryl(6-12C)alkyl(1-8C), aryl(6-12C)alkenyl(2-8C), or aryl(6-12C)alkynyl(2-8C), plus the heteroforms. A typical example is phenylmethyl, commonly referred to as benzyl.

Typical optional substituents on aromatic or heteroaromatic groups include independently halo, CN, NO₂, CF₃, OCF₃, COOR′, CONR′₂, OR′, SR′, SOR′, SO₂R′, NR′₂, NR′(CO)R′, NR′C(O)OR′, NR′C(O)NR′₂, NR′SO₂NR′₂, or NR′SO₂R′, wherein each R′ is independently H or an optionally substituted group selected from alkyl, alkenyl, alkynyl, heteroalkyl, heteroalkenyl, heteroalkynyl, heteroaryl, and aryl (all as defined above); or the substituent may be an optionally substituted group selected from alkyl, alkenyl, alkynyl, heteroalkyl, heteroalkenyl, heteroalkynyl, aryl, heteroaryl, O-aryl, O-heteroaryl and arylalkyl.

Optional substituents on a non-aromatic group, are typically selected from the same list of substituents suitable for aromatic or heteroaromatic groups and may further be selected from ═O and ═NOR′ where R′ is H or an optionally substituted group selected from alkyl, alkenyl, alkynyl, heteroalkyl, heteroalkenyl, heteroalkynyl, heteroaryl, and aryl (all as defined above).

Halo may be any halogen atom, especially F, Cl, Br, or I, and more particularly it is fluoro, chloro or bromo and even more particularly it is fluoro or chloro.

In general, any alkyl, alkenyl, alkynyl, or aryl (including all heteroforms defined above) group contained in a substituent may itself optionally be substituted by additional substituents. The nature of these substituents is similar to those recited with regard to the substituents on the basic structures above. Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments, and is not included. However, alkyl substituted by aryl, amino, halo and the like would be included.

A is C(O)NH or NHC(O). “p” is 0 or 1 indicating that X is present when n is 1 and X is absent when p is 0. X is an optionally substituted alkylene (1-4C), heteroalkylene (2-4C), alkenylene (2-4C), or heteralkenylene (2-4C). In more particular embodiments X is absent (i.e. n=0) or X is an optionally substituted alkylene (1-2C) or X is an optionally substituted alkenylene(2C). For example, in more particular embodiments, X is methylene. When X is present, the optional substituents on X are as defined above, however, in particular embodiments X may be unsubstituted.

“m” and “n” may each be 0 or 1. In more particular embodiments, at least one of m and n must be 1. In concurrent or alternate embodiments, A is NHC(O) if n is 0.

Ar is an optionally substituted aryl (6-10C) or heteroaryl (5-12 ring members). In particular embodiments, Ar is an optionally substituted phenyl, oxadiazolyl, thiazolyl, pyridinyl, or isoxazolyl. In more particular embodiments, Ar is an optionally substituted phenyl. Optional substituents on Ar are as defined above, however, in more particular embodiments such optional substituents may independently be selected from fluoro, chloro, trifluoromethyl, methyl, ethyl, trifluoromethoxy, t-butyl, t-butyloxy, methoxy, phenyl, or tolyl.

Each Y is independently H, SR′, SOR′, SO₂R′, wherein each R′ is independently H or an optionally substituted group selected from alkyl, alkenyl, alkynyl, heteroalkyl, heteroalkenyl, heteroalkynyl; or each Y is an optionally substituted group selected from alkyl, alkenyl, alkynyl, heteroalkyl, heteroalkenyl,heteralkynyl, aryl-alkyl, heteroaryl-alkyl; or two Y may together form an optionally substituted heterocyclic ring. In some embodiments, at least one Y is a hydrogen whereas in other embodiments both Y are hydrogen. In many embodiments, at least one Y is an alkyl, heteroalkyl, arylalkyl or heteroarylalkyl. In many embodiments, a carbonyl in Y is adjacent to the N. In some embodiments, two Y together form an optionally substituted heterocyclic ring, such as a pyrrolidinyl, piperidinyl, oxazolidinyl, morpholino. In more particular embodiments, the two Y groups together form an optionally substituted 2-oxooxazolidinyl.

In some preferred embodiments, the compound of the invention is of formula (2) as follows:

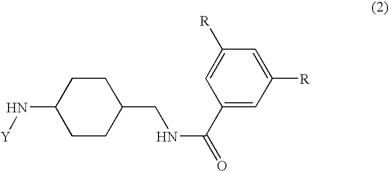

or a pharmaceutically acceptable salt or conjugate thereof, wherein Y is as defined above and each R is independently H, fluoro, chloro, trifluoromethyl, methyl, ethyl, trifluoromethoxy, t-butyl, t-butyloxy or methoxy.

In some preferred embodiments, two or more of the particularly described groups are combined into one compound: it is often suitable to combine one of the specified embodiments of one feature as described above with a specified embodiment or embodiments of one or more other features as described above. For example, a specified embodiment includes a compound of formula (1) with Ar equal to phenyl, and another specified embodiment has n equal to 1. Thus one preferred embodiment combines both of these features together, i.e., Ar is phenyl in combination with n=1. In some specific embodiments, one Y is H and in others A is NHC(O). Thus additional preferred embodiments include Y as H in combination with any of the preferred combinations set forth above; other preferred combinations include A as NHC(O) in combination with any of the preferred combinations set forth above.

The compounds of the invention may be in the form of pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

In some cases, the compounds of the invention contain one or more chiral centers. The invention includes each of the isolated stereoisomeric forms as well as mixtures of stereoisomers in varying degrees of chiral purity, including racemic mixtures. It also encompasses the various diastereomers and tautomers that can be formed.

Compounds of formula (1) are also useful for the manufacture of a medicament useful to treat conditions characterized by undesired T-type calcium channel activities.

In addition, the compounds of the invention may be coupled through conjugation to substances designed to alter the pharmacokinetics, for targeting, or for other reasons. Thus, the invention further includes conjugates of these compounds. For example, polyethylene glycol is often coupled to substances to enhance half-life; the compounds may be coupled to liposomes covalently or noncovalently or to other particulate carriers. They may also be coupled to targeting agents such as antibodies or peptidomimetics, often through linker moieties. Thus, the invention is also directed to the compounds of formula (1) when modified so as to be included in a conjugate of this type.

### Modes of Carrying Out the Invention

The compounds of formula (1) are useful in the methods of the invention and, while not bound by theory, are believed to exert their desirable effects through their ability to modulate the activity of calcium channels, particularly the activity of T-type calcium channels. This makes them useful for treatment of certain conditions where modulation of T-type calcium channels is desired, including: cardiovascular disease; epilepsy; diabetes; cancer; pain, including both chronic and acute pain; sleep disorders; Parkinson's disease; psychosis such as schizophrenia; overactive bladder; renal disease, neuroprotection, addiction and male birth control.

Cardiovascular disease as used herein includes but is not limited to hypertension, pulmonary hypertension, arrhythmia (such as atrial fibrillation and ventricular fibrillation), congestive heart failure, and angina pectoris.

Epilepsy as used herein includes but is not limited to partial seizures such as temporal lobe epilepsy, absence seizures, generalized seizures, and tonic/clonic seizures.

Cancer as used herein includes but is not limited to breast carcinoma, neuroblastoma, retinoblastoma, glioma, prostate carcinoma, esophageal carcinoma, fibrosarcoma, colorectal carcinoma, pheochromocytoma, adrenocarcinoma, insulinoma, lung carcinoma, melanoma, and ovarian cancer.

Acute pain as used herein includes but is not limited to nociceptive pain and post-operative pain. Chronic pain includes but is not limited by: peripheral neuropathic pain such as post-herpetic neuralgia, diabetic neuropathic pain, neuropathic cancer pain, failed back-surgery syndrome, trigeminal neuralgia, and phantom limb pain; central neuropathic pain such as multiple sclerosis related pain, Parkinson disease related pain, post-stroke pain, post-traumatic spinal cord injury pain, and pain in dementia; musculoskeletal pain such as osteoarthritic pain and fibromyalgia syndrome; inflammatory pain such as rheumatoid arthritis and endometriosis; headache such as migraine, cluster headache, tension headache syndrome, facial pain, headache caused by other diseases; visceral pain such as interstitial cystitis, irritable bowel syndrome and chronic pelvic pain syndrome; and mixed pain such as lower back pain, neck and shoulder pain, burning mouth syndrome and complex regional pain syndrome.

For greater certainty, in treating osteoarthritic pain, joint mobility will also improve as the underlying chronic pain is reduced. Thus, use of compounds of the present invention to treat osteoarthritic pain inherently includes use of such compounds to improve joint mobility in patients suffering from osteoarthritis.

Addiction includes but is not limited to dependence, withdrawal and/or relapse of cocaine, opioid, alcohol and nicotine

It is known that calcium channel activity is involved in a multiplicity of disorders, and particular types of channels are associated with particular conditions. The association of T-type channels in conditions associated with neural transmission would indicate that compounds of the invention which target T-type receptors are most useful in these conditions. Many of the members of the genus of compounds of formula (1) exhibit high affinity for T-type channels. Thus, as described below, they are screened for their ability to interact with T-type channels as an initial indication of desirable function. It is particularly desirable that the compounds exhibit IC₅₀ values of <1 μM. The IC₅₀ is the concentration which inhibits 50% of the calcium, barium or other permeant divalent cation flux at a particular applied potential.

In order to be maximally useful in treatment, it is also helpful to assess the side reactions which might occur. Thus, in addition to being able to modulate a particular calcium channel, it is desirable that the compound has very low activity with respect to the hERG K⁺ channel which is expressed in the heart. Compounds that block this channel with high potency may cause reactions which are fatal. Thus, for a compound that modulates the calcium channel, it is preferred that the hERG K⁺ channel is not inhibited. Some inhibition of the hERG K⁺ channel may be tolerated in a drug as long as the compound is sufficiently selective for the target of interest over the hERG K⁺ channel. For example, 10 fold selectivity of a T-type calcium channel over the hERG K⁺ channel would be beneficial and more preferably 30 fold selectivity or 100 fold selectivity.

Similarly, it would be undesirable for the compound to inhibit cytochrome p450 since this enzyme is required for drug detoxification. Finally, the compound will be evaluated for calcium ion channel type specificity by comparing its activity among the various types of calcium channels, and specificity for one particular channel type is preferred. The compounds which progress through these tests successfully are then examined in animal models as actual drug candidates.

The compounds of the invention modulate the activity of calcium channels; in general, said modulation is the inhibition of the ability of the channel to transport calcium. As described below, the effect of a particular compound on calcium channel activity can readily be ascertained in a routine assay whereby the conditions are arranged so that the channel is activated, and the effect of the compound on this activation (either positive or negative) is assessed. Typical assays are described hereinbelow in Example 15.

Libraries and Screening

The compounds of the invention can be synthesized individually using methods known in the art per se, or as members of a combinatorial library.

Synthesis of combinatorial libraries is now commonplace in the art. Suitable descriptions of such syntheses are found, for example, in Wentworth, Jr., P., et al., Current Opinion in Biol. (1993) 9:109-115; Salemme, F. R., et al., Structure (1997) 5:319-324. The libraries contain compounds with various substituents and various degrees of unsaturation, as well as different chain lengths. The libraries, which contain, as few as 10, but typically several hundred members to several thousand members, may then be screened for compounds which are particularly effective against a specific subtype of calcium channel, e.g., the N-type channel. In addition, using standard screening protocols, the libraries may be screened for compounds that block additional channels or receptors such as sodium channels, potassium channels and the like.

Methods of performing these screening functions are well known in the art. These methods can also be used for individually ascertaining the ability of a compound to agonize or antagonize the channel. Typically, the channel to be targeted is expressed at the surface of a recombinant host cell such as human embryonic kidney cells. The ability of the members of the library to bind the channel to be tested is measured, for example, by the ability of the compound in the library to displace a labeled binding ligand such as the ligand normally associated with the channel or an antibody to the channel. More typically, ability to antagonize the channel is measured in the presence of calcium, barium or other permeant divalent cation and the ability of the compound to interfere with the signal generated is measured using standard techniques. In more detail, one method involves the binding of radiolabeled agents that interact with the calcium channel and subsequent analysis of equilibrium binding measurements including, but not limited to, on rates, off rates, K_{d} values and competitive binding by other molecules.

Another method involves the screening for the effects of compounds by electrophysiological assay whereby individual cells are impaled with a microelectrode and currents through the calcium channel are recorded before and after application of the compound of interest.

Another method, high-throughput spectrophotometric assay, utilizes loading of the cell lines with a fluorescent dye sensitive to intracellular calcium concentration and subsequent examination of the effects of compounds on the ability of depolarization by potassium chloride or other means to alter intracellular calcium levels.

As described above, a more definitive assay can be used to distinguish inhibitors of calcium flow which operate as open channel blockers, as opposed to those that operate by promoting inactivation of the channel or as resting channel blockers. The methods to distinguish these types of inhibition are more particularly described in the examples below. In general, open-channel blockers are assessed by measuring the level of peak current when depolarization is imposed on a background resting potential of about −100 mV in the presence and absence of the candidate compound. Successful open-channel blockers will reduce the peak current observed and may accelerate the decay of this current. Compounds that are inactivated channel blockers are generally determined by their ability to shift the voltage dependence of inactivation towards more negative potentials. This is also reflected in their ability to reduce peak currents at more depolarized holding potentials (e.g., −70 mV) and at higher frequencies of stimulation, e.g., 0.2 Hz vs. 0.03 Hz. Finally, resting channel blockers would diminish the peak current amplitude during the very first depolarization after drug application without additional inhibition during the depolarization.

Accordingly, a library of compounds of formula (1) can be used to identify a compound having a desired combination of activities that includes activity against at least one type of calcium channel. For example, the library can be used to identify a compound having a suitable level of activity on T-type calcium channels while having minimal activity on HERG K+channels.

Utility and Administration

For use as treatment of human and animal subjects, the compounds of the invention can be formulated as pharmaceutical or veterinary compositions. Depending on the subject to be treated, the mode of administration, and the type of treatment desired—e.g., prevention, prophylaxis, therapy; the compounds are formulated in ways consonant with these parameters. A summary of such techniques is found in Remington's Pharmaceutical Sciences, latest edition, Mack Publishing Co., Easton, Pa., incorporated herein by reference.

In general, for use in treatment, the compounds of formula (1) may be used alone, as mixtures of two or more compounds of formula (1) or in combination with other pharmaceuticals. An example of other potential pharmaceuticals to combine with the compounds of formula (1) would include pharmaceuticals for the treatment of the same indication but having a different mechanism of action from T-type calcium channel blocking. For example, in the treatment of pain, a compound of formula (1) may be combined with another pain relief treatment such as an NSAID, or a compound which selectively inhibits COX-2, or an opioid, or an adjuvant analgesic such as an antidepressant. Another example of a potential pharmaceutical to combine with the compounds of formula (1) would include pharmaceuticals for the treatment of different yet associated or related symptoms or indications. Depending on the mode of administration, the compounds will be formulated into suitable compositions to permit facile delivery.

The compounds of the invention may be prepared and used as pharmaceutical compositions comprising an effective amount of at least one compound of formula (1) admixed with a pharmaceutically acceptable carrier or excipient, as is well known in the art. Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, preservatives and the like. The compounds can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, as is understood in the art.

For administration to animal or human subjects, the dosage of the compounds of the invention is typically 0.01-15 mg/kg, preferably 0.1-10 mg/kg. However, dosage levels are highly dependent on the nature of the condition, drug efficacy, the condition of the patient, the judgment of the practitioner, and the frequency and mode of administration. Optimization of the dosage for a particular subject is within the ordinary level of skill in the art.

Synthesis of the Invention Compounds

The following reaction schemes and examples are intended to illustrate the synthesis of a representative number of compounds. Accordingly, the following examples are intended to illustrate but not to limit the invention. Additional compounds not specifically exemplified may be synthesized using conventional methods in combination with the methods described hereinbelow.

### EXAMPLE 1

### Synthesis of Synthesis of N-((trans-4-(2-hydroxy-3,3-dimethylbutylamino)cyclohexyl)methyl)-3,5 -bis(trifluoromethyl)benzamide (Compound 3)

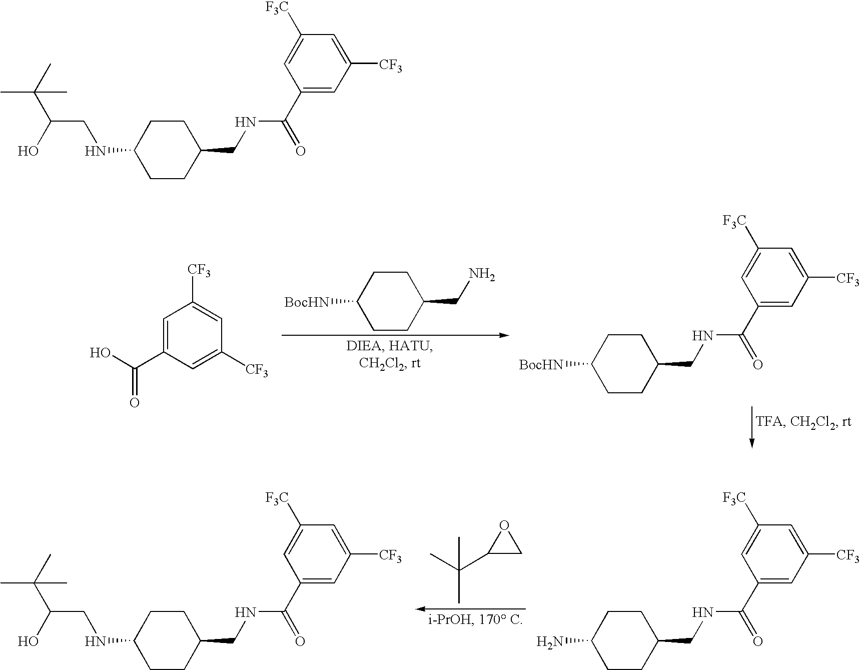

### A. Synthesis of tert-butyl trans-4-((3,5-bis(trifluoromethyl)benzamido)methyl)cyclohexylcarbamate

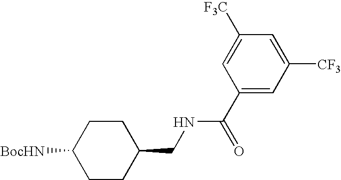

To a solution of 3,5-bis(trifluoromethyl)benzoic acid (2.0 g, 7.8 mmol) in CH₂Cl₂ (50 mL) was added DIEA (4.0 mL, 23.4 mmol), tert-butyl trans-4-(aminomethyl)cyclohexylcarbamate (1.8 g, 7.8 mmol), and HATU (3.8 g, 10.1 mmol). The resultant mixture was stirred at RT overnight. The reaction was concentrated and sat. NaHCO₃ aq (50 mL) was added. The aqueous layer was extracted with EtOAc (2×50 mL) and the combined organic layers was dried over Na₂SO₄, filtered, and concentrated to give crude tert-butyl trans-4-((3,5-bis(trifluoromethyl)benzamido)methyl)cyclohexylcarbamate. Purification by Biotage using a mixture of EtOAc:hexanes (1:4) provided pure tert-butyl trans-4-((3,5-bis(trifluoromethyl)benzamido)methyl)cyclohexylcarbamate (2.1 g, 57%).

### B. Synthesis of -((trans-4-aminocyclohexyl)methyl)-3,5-bis(trifluoromethyl)benzamide

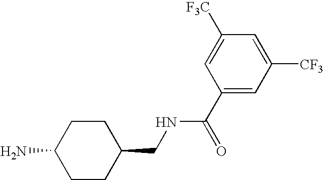

To a solution of N-((trans-4-aminocyclohexyl)methyl)-3,5-bis(trifluoromethyl)benzamide (2.1 g, 4.5 mmol) in CH₂Cl₂ (25 mL) was added trifluoroacetic acid (25 mL) and the resultant mixture was stirred at RT overnight. The reaction mixture was concentrated and sat. NaHCO₃ (50 mL) was added. The aqueous layer was extracted with EtOAc (2×50 mL) and the combined organic layers was dried over Na₂SO₄, filtered, and concentrated to give of N-((trans-4-aminocyclohexyl)methyl)-3,5-bis(trifluoromethyl)benzamide (1.25 g, 76%) which required no further purification.

### C. Synthesis of N-((trans-4-(2-hydroxy-3,3-dimethylbutylamino)cyclohexyl)methyl)-3,5-bis(trifluoromethyl)benzamide (Compound 3)

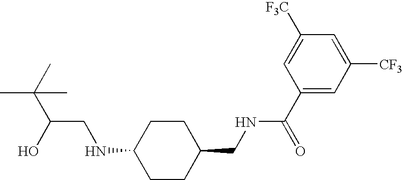

A solution of N-((trans-4-aminocyclohexyl)methyl)-3,5-bis(trifluoromethyl)benzamide (0.1 g, 0.27 mmol) and 2-tert-butyloxirane (54 mg, 0.54 mmol) in i-PrOH (0.5 mL) was irradiated under microwave conditions at 170° C. for 30 min. The reaction mixture was concentrated and the crude residue was purified by HiTOPs to give N-((trans-4-(2-hydroxy-3,3-dimethylbutylamino)cyclohexyl)methyl)-3,5-bis(trifluoromethyl)benzamide.

### EXAMPLE 2

### Synthesis of N-((trans-4-(2-hydroxybenzylamino)cyclohexyl)methyl)-3,5-bis(trifluoromethyl)benzamide (Compound 25)

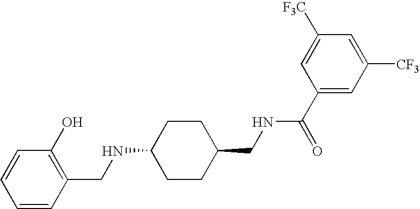

To a solution of N-((trans-4-aminocyclohexyl)methyl)-3,5-bis(trifluoromethyl)benzamide (0.1 g, 0.27 mmol) and 2-hydroxybenzaldehyde (33 mg, 0.27 mmol) in CH₂Cl₂ (5 mL) was added AcOH (0.1 mL) followed by NaBH(OAc)3 (80 mg, 0.38 mmol). The reaction mixture was stirred at RT overnight. The reaction was quenched with sat. NH₄Cl aq. (5 mL) and the organic layer was separated and concentrated. The resultant residue was purified by HiTOPs to give pure N-((trans-4-(2-hydroxybenzylamino)cyclohexyl)methyl)-3,5-bis(trifluoromethyl)benzamide.

### EXAMPLE 3

### Synthesis of N-((trans-4-(2-(tert-butylamino)-2-oxoethylamino)cyclohexyl)methyl)-3,5-bis(trifluoromethyl)benzamide (Compound 55)

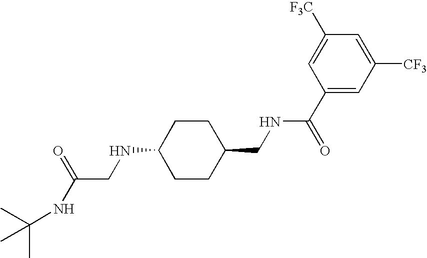

A solution of N-((trans-4-aminocyclohexyl)methyl)-3,5-bis(trifluoromethyl)benzamide (50 mg, 0.15 mmol), N-tert-butyl-2-chloroacetamide (22 mg, 0.15 mmol) and DIEA (0.03 mL, 0.16 mmol) in DMF (0.5 mL) was stirred at RT overnight. The reaction mixture was concentrated and the resultant residue was purified by HiTOPs to give N-((trans-4-(3 ,3-dimethyl-2-oxobutylamino)cyclohexyl)methyl)-3,5-bis(trifluoromethyl)benzamide.

### EXAMPLE 4

### Synthesis of N-((trans-4-(2-(2-cyclopropylacetamido)ethylamino)cyclohexyl)methyl)-3,5-bis(trifluoromethyl)benzamide (Compound 103)

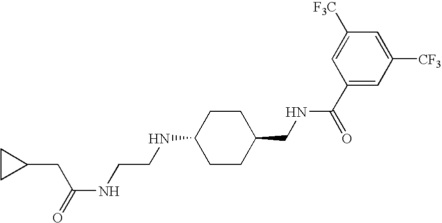

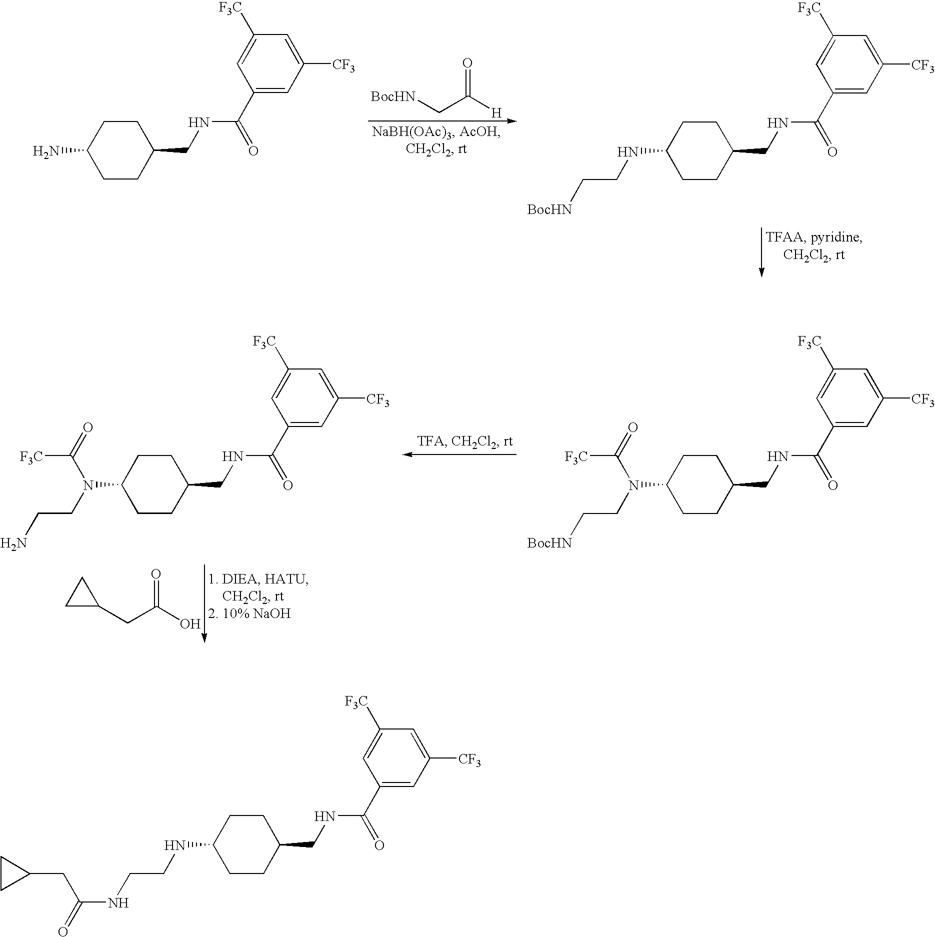

### A. Synthesis of tert-butyl 2-(trans-4-((3,5-bis(trifluoromethyl)benzamido)methyl)cyclohexylamino)ethylcarbamate

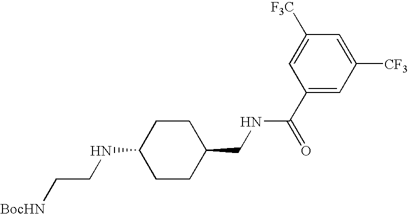

To a solution N-((trans-4-aminocyclohexyl)methyl)-3,5-bis(trifluoromethyl)benzamide (1.5 g, 4.2 mmol) and tert-butyl 2-oxoethylcarbamate (0.67 g, 4.2 mmol) in CH₂Cl₂ (8 mL) was added AcOH (1 mL) followed by NaBH(OAc)3 (1.15 g, 5.46 mmol). The reaction mixture was stirred at RT overnight. The reaction was quenched with sat. NH₄Cl aq. (15 mL) and the organic layer was separated and concentrated. The resultant residue was purified by Biotage using 5% MeOH/CH₂Cl₂ gradient. Pure fractions were pooled to give tert-butyl 2-(trans-4-((3,5-bis(trifluoromethyl)benzamido)methyl)cyclohexylamino)ethylcarbamate (1.52 g, 71%).

### B. Synthesis of tert-butyl 2-(N-(trans-4-((3,5-bis(trifluoromethyl)benzamido)methyl)cyclohexyl)-2,2,2-trifluoroacetamido)ethylcarbamate

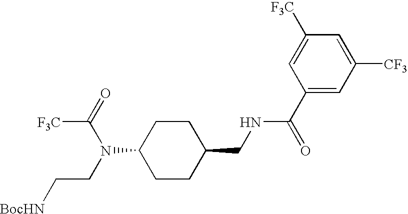

To a solution of tert-butyl 2-(trans-4-((3,5-bis(trifluoromethyl)benzamido)methyl)cyclohexylamino)ethylcarbamate (0.1 g, 0.2 mmol) in CH₂Cl₂ (10 mL) was added pyridine (2 mL) followed by trifluoroacetic anhydride (1 mL). The reaction mixture was stirred at RT overnight. The reaction mixture was concentrated and the resultant residue was partitioned between sat. NaHCO₃ aq. (20 mL) and CH₂Cl₂ (2×10 mL). The combined organic layers were dried over Na₂SO₄, filtered and concentrated. Purification by Biotage using a mixture of EtOAc:hexanes (1:4) provided tert-butyl 2-(N-(trans-4-((3,5-bis(trifluoromethyl)benzamido)methyl)cyclohexyl)-2,2,2-trifluoroacetamido)ethylcarbamate (69 mg, 57%).

### C. Synthesis of N-((trans-4-(N-(2-aminoethyl)-2,2,2-trifluoroacetamido)cyclohexyl)methyl)-3,5-bis(trifluoromethyl)benzamide

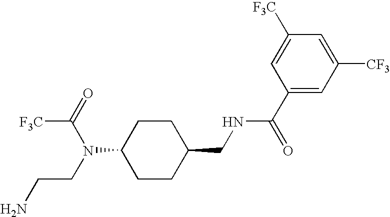

To a solution of tert-butyl 2-(N-(trans-4-((3,5-bis(trifluoromethyl)benzamido)methyl)cyclohexyl)-2,2,2-trifluoroacetamido)ethylcarbamate (69 mg, 0.11 mol) in CH₂Cl₂ (3 mL) was added trifluoroacetic acid (3 mL). The reaction mixture was stirred at RT overnight. The reaction was concentrated and the crude residue was partitioned between sat. NaHCO₃ aq. (10 mL) and EtOAc (2×10 mL). The combined organic layers were dried over Na₂SO₄, filtered and concentrated to give N-((trans-4-(N-(2-aminoethyl)-2,2,2-trifluoroacetamido)cyclohexyl)methyl)-3,5-bis(trifluoromethyl)benzamide (54 mg, 100%) which required no further purification.

### D. Synthesis of N-((trans-4-(2-(2-cyclopropylacetamido)ethylamino)cyclohexyl)methyl)-3,5-bis(trifluoromethyl)benzamide (Compound 103)

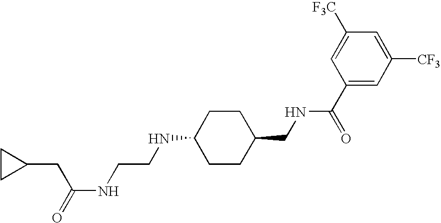

To a solution of N-((trans-4-(N-(2-aminoethyl)-2,2,2-trifluoroacetamido)cyclohexyl)methyl)-3,5-bis(trifluoromethyl)benzamide (80 mg, 0.16 mmol) in CH₂Cl₂ (5 mL) was added DIEA (0.08 mL, 0.47 mmol) followed by HATU (80 mg, 0.2 mmol). After stirring at RT for 16 h, the reaction mixture was concentrated and the resultant residue was redissolved in MeOH (2 mL). A solution of 10% NaOH aq. was then added and the reaction was stirred at RT overnight. MeOH was removed and the aqueous layer was extracted with CH₂Cl₂ (2 mL). The organic layer was concentrated and the resultant residue was purified by HiTOPs to give N-((trans-4-(2-(2-cyclopropylacetamido)ethylamino)cyclohexyl)methyl)-3,5-bis(trifluoromethyl)benzamide.

### EXAMPLE 5

### Synthesis of butyl trans-4-(2-(3,5-bis(trifluoromethyl)phenylamino)-2-oxoethyl)cyclohexylcarbamate (Compound 177)

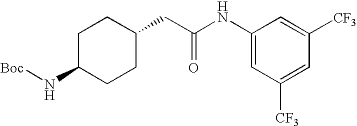

A solution of 2-(trans-4-(tert-butoxycarbonylamino)cyclohexyl)acetic acid (5.00 g, 19.4 mmol), 3,5-bis(trifluoromethyl)aniline (3.34 mL, 21.4 mmol), EDC (3.32 g, 21.4 mmol), TEA (2.98 mL, 21.4 mmol), and DMAP (cat.) in CH₂Cl₂ (60 mL) was stirred at room temperature for two days. The reaction mixture was then concentrated to ˜30 mL, diluted with EtOAc, washed with saturated aqueous NH₄Cl (2 times), dried with Na₂SO₄, filtered, and the solvent was removed in vacuo. The resulting crude material was purified by automated flash chromatography (pet ether: EtOAc) to provide tert-butyl trans-4-(2-(3,5-bis(trifluoromethyl)phenylamino)-2-oxoethyl)cyclohexylcarbamate (2.57 g, 28%)..

### EXAMPLE 6

### Synthesis of 2-(trans-4-aminocyclohexyl)-N-(3,5-bis(trifluoromethyl)phenyl)acetamide (Compound 178)

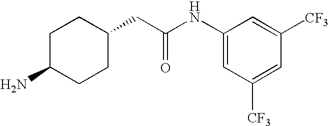

A solution of tert-butyl trans-4-(2-(3,5-bis(trifluoromethyl)phenylamino)-2-oxoethyl)cyclohexylcarbamate (2.57 g, 5.49 mmol) in CH₂Cl₂ was treated with 2M HCl in Et₂O (40 mL) and stirred overnight. The solvent was removed under reduced pressure, the residue was dissolved with EtOAc, washed with saturated aqueous NaHCO₃, dried with Na₂SO₄, filtered, and the solvent was again removed to provide 2-(trans-4-aminocyclohexyl)-N-(3,5-bis(trifluoromethyl)phenyl)acetamide in excellent yield.

### EXAMPLE 7

### Synthesis of (S)—N-(cis-4-(2-(3,5-bis(trifluoromethyl)phenylamino)-2-oxoethyl)cyclohexyl)-2-hydroxy-3,3 -dimethylbutanamide (Compound 108)

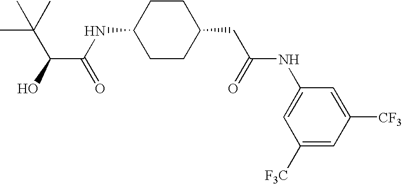

A solution of 2-(cis-4-aminocyclohexyl)-N-(3,5-bis(trifluoromethyl)phenyl)acetamide (140 mg, 0.380 mmol), (S)-2-hydroxy-3,3-dimethylbutanoic acid (151 mg, 1.14 mmol), HATU (433 mg, 1.14 mmol), and TEA (212 μL, 1.52 mmol) in DMF (5 mL) was stirred at room temperature for 2 days. The reaction mixture was diluted with EtOAc, washed with saturated aqueous NaHCO₃ (2 times), dried with Na₂SO₄, filtered, and the solvent was removed under reduced pressure. The crude residue was prepurified by automated flash chromatography (MeOH:CH₂Cl₂), then purified by reverse phase HPLC to provide (S)—N-(cis-4-(2-(3,5-bis(trifluoromethyl)phenylamino)-2-oxoethyl)cyclohexyl)-2-hydroxy-3,3-dimethylbutanamide.

### EXAMPLE 8

### Synthesis of (2R,5S)—N-(trans-4-((3,5-bis(trifluoromethyl)benzamido)methyl)cyclohexyl)-5-phenylpyrrolidine-2-carboxamide (Compound 137)

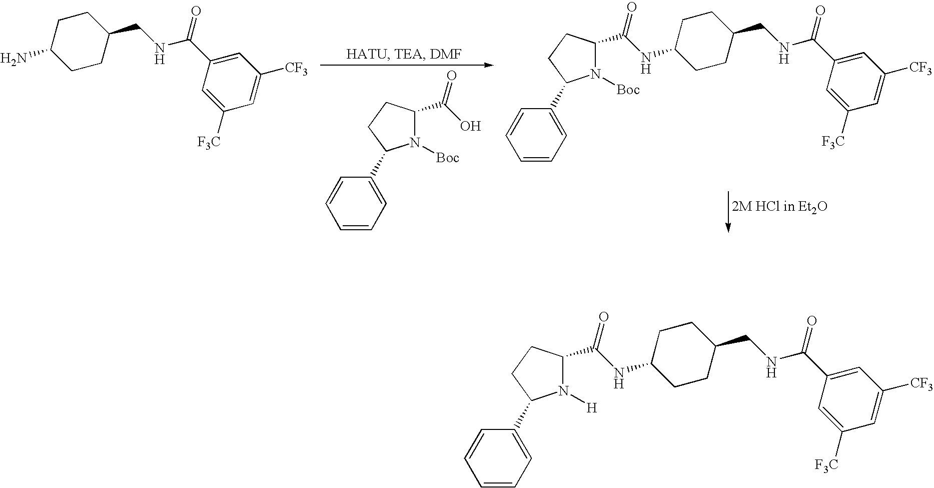

A solution of N-((trans-4-aminocyclohexyl)methyl)-3,5-bis(trifluoromethyl)benzamide (50 mg, 0.14 mmol), (2R,5S)-1-(tert-butoxycarbonyl)-5-phenylpyrrolidine-2-carboxylic acid (47 mg, 0.16 mmol), HATU (77 mg, 0.20 mmol), and TEA (95 μL, 0.68 mmol) in DMF (2.5 mL) was stirred at room temperature for 2 days. The reaction mixture was diluted with saturated aqueous NaHCO₃ (5 mL), EtOAc (5 mL), and mixed vigourously. The layers were allowed to separate, and then cooled to −20° C. in the freezer. Once the aqueous layer had frozen, the organic layer was poured off and the solvent was removed under reduced pressure to provide (2R,5S)-tert-butyl 2-(trans-4-((3,5-bis(trifluoromethyl)benzamido)methyl)cyclohexylcarbamoyl)-5 -phenylpyrrolidine-1-carboxylate as a crude oil. The crude oil was dissolved with CH₂Cl₂ (1 mL) and treated with 2M HCl in Et₂O (3 mL). After stirring overnight, the solvent was removed in vacuo and the residue was purified by reverse phase HPLC to provide the product, (2R,5S)—N-(trans-4-((3,5-bis(trifluoromethyl)benzamido)methyl)cyclohexyl)-5-phenylpyrrolidine-2-carboxamide.

### EXAMPLE 9

### Synthesis of N-(3,5-bis(trifluoromethyl)phenyl)-2-(cis-4-(2-hydroxy-3,3-dimethylbutylamino)cyclohexyl)acetamide (Compound 150)

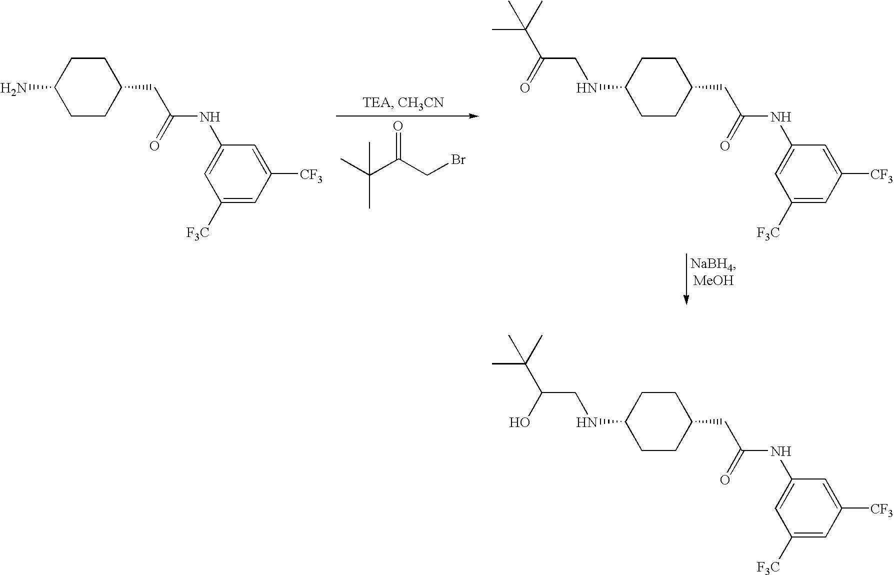

### A. Synthesis of N-(3,5-bis(trifluoromethyl)phenyl)-2-(cis-4-(3,3-dimethyl-2-oxobutylamino)cyclohexyl)acetamide

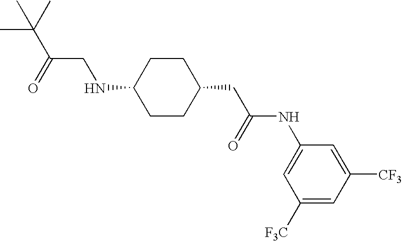

A solution of 2-(cis-4-aminocyclohexyl)-N-(3,5-bis(trifluoromethyl)phenyl)acetamide (241 mg, 0.645 mmol) and TEA (182 μL, 1.31 mmol) in CH3CN (20 mL) was treated with 1-bromo-3,3-dimethylbutan-2-one (88 μL, 0.65 mmol). After stirring for two days at room temperature, the reaction mixture was diluted with EtOAc, washed with saturated aqueous NaHCO3, dried with Na2SO4, filtered, and the solvent was removed under reduced pressure. The residue was purified by automated flash chromatography to provide the product, N-(3,5-bis(trifluoromethyl)phenyl)-2-(cis-4-(3,3-dimethyl-2-oxobutylamino)cyclohexyl)acetamide, in good yield.

### B. Synthesis of N-(3,5-bis(trifluoromethyl)phenyl)-2-(cis-4-(2-hydroxy-3,3-dimethylbutylamino)cyclohexyl)acetamide (Compound 150)

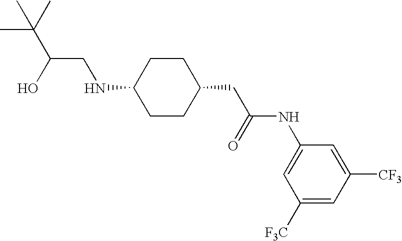

A solution of N-(3,5-bis(trifluoromethyl)phenyl)-2-(cis-4-(3,3-dimethyl-2-oxobutylamino)cyclohexyl)acetamide (107 mg, 0.229 mmol) in MeOH (1.5 mL) was treated with NaBH₄ (17 mg, 0.46 mmol). The reaction was stirred for 40 minutes, then quenched with water, and extracted with EtOAc. The organic layer was dried with Na₂SO₄, filtered, and the solvent removed in vacuo to provide the crude product. The residue was purified by reverse phase HPLC to yield the product, N-(3,5-bis(trifluoromethyl)phenyl)-2-(cis-4-(2-hydroxy-3,3-dimethylbutylamino)cyclohexyl)acetamide.

### EXAMPLE 10

### Synthesis of N-(4-p-tolylthiazol-2-yl)-2-(cis-4-(3,3,3-trifluoro-2-hydroxy-2-(trifluoromethyl)propylamino)cyclohexyl)acetamide (Compound 152)

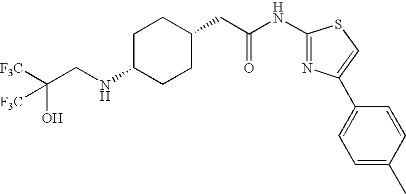

To a solution of 2-(cis-4-aminocyclohexyl)-N-(4-p-tolylthiazol-2-yl)acetamide (192 mg, 0.58 mmol) in i-PrOH (5 mL) was added 2,2-bis(trifluoromethyl)oxirane (0.2 mL, 1.1 mmol). The resulting mixture was subjected to microwave 1 h at 100° C. and then concentrated. Purification by HiTOPs gave N-(4-p-tolylthiazol-2-yl)-2-(cis-4-(3,3,3-trifluoro-2-hydroxy-2-(trifluoromethyl)propylamino)cyclohexyl)acetamide as final compound.

### EXAMPLE 11

### Synthesis of 2-(cis-4-(2-oxo-5,5-bis(trifluoromethyl)oxazolidin-3-yl)cyclohexyl)-N-(4-p-tolylthiazol-2-yl)acetamide (Compound 176)

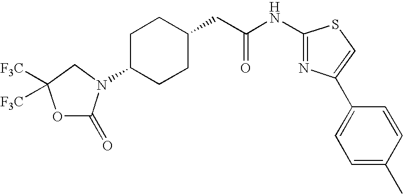

To a solution of N-(4-p-tolylthiazol-2-yl)-2-(cis-4-(3,3,3-trifluoro-2-hydroxy-2-(trifluoromethyl)propylamino)cyclohexyl)acetamide (133 mg, 0.26 mmol) and DIPEA (0.07 mL, 0.38 mmol) in CH₂Cl₂ (2 mL) was added triphosgen (38 mg, 0.25 mmol) at room temperature. The resulting mixture was allowed to stir at room temperature for 2 hours then diluted with ethyl acetate. The organic was washed by sat. NaHCO₃, then brine and dried over Na₂SO₄. Evaporation of solvent and purification by HiTOPs gave 2-(cis-4-(2-oxo-5,5-bis-(trifluoromethyl)-oxazolidin-3-yl)-cyclohexyl)-N-(4-p-tolylthiazol-2-yl)acetamide as desired product.

### EXAMPLE 12

### Synthesis of 3,5-bis(trifluoromethyl)-N-((trans-4-(2-(trifluoromethylsulfonamido)ethylamino)cyclohexyl)methyl)benzamide (Compound 155)

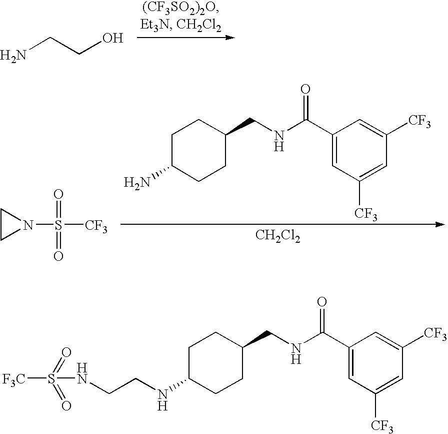

At −78° C., to a solution of 2-aminoethanol (0.84 g, 13.8 mmol) and triethylamine (3.85 mL, 27.7 mmol) in CH₂Cl₂ (100 mL) was added slowly trifluoromethanesulfonic anhydride (8.4 g, 29.8 mmol). The reaction mixture was stirred at −78° C. for 2 hrs, warmed to −40° C. and stirred at −40° C. overnight. The reaction mixture was then diluted with CH₂Cl₂ (50 mL), washed with 0.1N aqueous HCl (2×150 mL) and saturated aqueous NaHCO₃ (150 mL) and dried over anhydrous Na₂SO₄. After filtration, the filtrate was concentrated at 0° C. to 25 mL. 1 mL of this stock solution (˜0.6 mmol) was then added to pre-dissolved N-((trans-4-aminocyclohexyl)methyl)-3,5-bis(trifluoromethyl)benzamide (0.2 g, 0.55 mmol) in 3 mL of CH₂Cl₂. The mixture was stirred at RT for 3 h. The reaction mixture was concentrated and the resultant residue was purified by HiTOPs to give pure 3,5-bis(trifluoromethyl)-N-((trans-4-(2(trifluoromethylsulfonamido)ethylamino)cyclohexyl)methyl)benzamide.

### EXAMPLE 13

### Synthesis of N-((trans-4-((2-(tert-butylamino)-2-oxoethylamino)methyl)cyclohexyl)methyl)-3,5-bis(trifluoromethyl)benzamide (Compound 164)

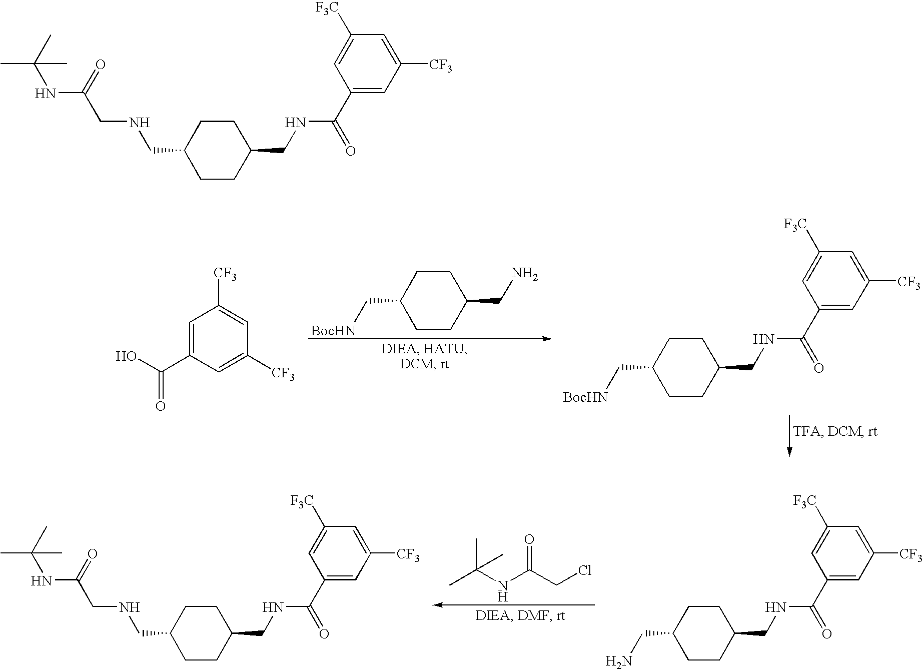

### A. Synthesis of tert-butyl(trans-4-((3,5-bis(trifluoromethyl)benzamido)methyl)cyclohexyl)methylcarbamate

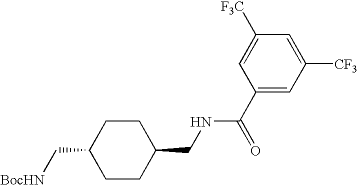

To a solution of 3,5-bis(trifluoromethyl)benzoic acid (2.3 g, 8.8 mmol) in DCM (50 mL) was added DIEA (4.6 mL, 26.4 mmol), tert-butyl (trans-4-(aminomethyl)cyclohexyl)methylcarbamate (2.1 g, 8.8 mmol), and HATU (4.3 g, 11.4 mmol). The resultant mixture was stirred at RT overnight. The reaction was concentrated and sat. NaHCO₃ aq (50 mL) was added. The aqueous layer was extracted with EtOAc (2×50 mL) and the combined organic layers was dried over Na₂SO₄, filtered, and concentrated to give crude tert-butyl trans-4-((3,5-bis(trifluoromethyl)benzamido)methyl)cyclohexylcarbamate. Purification by Biotage using a mixture of EtOAc:hexanes (1:4) provided 3.1 g (73%) of pure tert-butyl (trans-4-((3,5-bis(trifluoromethyl)benzamido)methyl)cyclohexyl)methylcarbamate.

### B. Synthesis of N-((trans-4-(aminomethyl)cyclohexyl)methyl)-3,5-bis(trifluoromethyl)benzamide

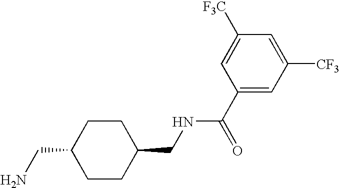

To a solution of tert-butyl(trans-4-((3,5-bis(trifluoromethyl)benzamido)methyl)cyclohexyl)methylcarbamate (3.1 g, 6.6 mmol) in DCM (25 mL) was added trifluoroacetic acid (25 mL) and the resultant mixture was stirred at RT overnight. The reaction mixture was concentrated and sat. NaHCO₃ (50 mL) was added. The aqueous layer was extracted with EtOAc (2×50 mL) and the combined organic layers was dried over Na₂SO₄, filtered, and concentrated. Purification by Biotage using a mixture of MeOH:DCM (2:98 to 1:5) afforded 1.8 g (75%) of N-((trans-4-(aminomethyl)cyclohexyl)methyl)-3,5-bis(trifluoromethyl)benzamide.

### C. Synthesis of N-((trans-4-((2-(tert-butylamino)-2-oxoethylamino)methyl)cyclohexyl)methyl)-3,5-bis(trifluoromethyl)benzamide (Compound 164)

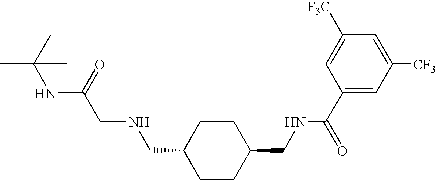

A solution of N-((trans-4-(aminomethyl)cyclohexyl)methyl)-3,5-bis(trifluoromethyl)benzamide (40 mg, 0.1 mmol), N-tert-butyl-2-chloroacetamide (15 mg, 0.1 mmol) and DIEA (0.017 mL, 0.1 mmol) in DMF (0.5 mL) was stirred ar RT overnight. The reaction mixture was concentrated and the resultant residue was purified by HiTOPs to give pure N-((trans-4-((2-(tert-butylamino)-2-oxoethylamino)methyl)cyclohexyl)methyl)-3,5-bis(trifluoromethyl)benzamide.

### EXAMPLE 14

Following the general procedures set forth in Examples 1-13, the following compounds listed in Table 1 below were prepared. Mass spectrometry was employed with the final compound and at various stages throughout the synthesis as a confirmation of the identity of the product obtained (M+1). For the mass spectrometric analysis, samples were prepared at an approximate concentration of 1 μg/mL in acetonitrile with 0.1% formic acid. Samples were then manually infused into an Applied Biosystems API3000 triple quadrupole mass spectrometer and scanned in Q1 in the range of 50 to 700 m/z.

TABLE 1

| | | | Mass |
|---|---|---|---|
| Cmpd | | | Spec |
| No. | Name | Structure | (m/z) |
| 1 | N-(cis-4-(2-hydroxy-3,3- dimethylbutylamino)cyclohexyl)methyl)- 3,5-bis(trifluoromethyl)benzamide | 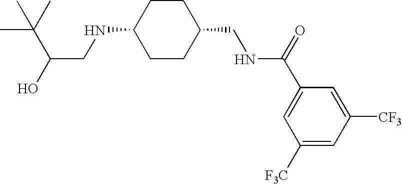 | 469.22 |
| 2 | 3-fluoro-N-((cis-4-(2-hydroxy-3,3- dimethylbutylamino)cyclohexyl)methyl)- 5-(trifluoromethyl)benzamide | 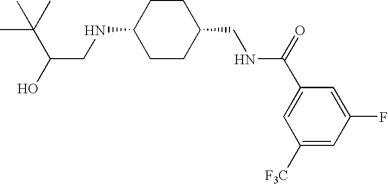 | 419.22 |
| 3 | N-((trans-4-(2-hydroxy-3,3- dimethylbutylamino)cyclohexyl)methyl)- 3,5-bis(trifluoromethyl)benzamide | 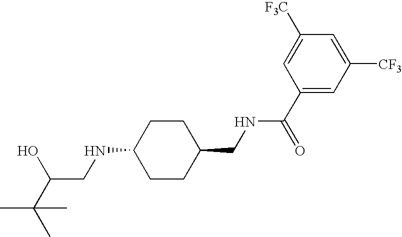 | 469.22 |
| 4 | N-((trans-4-(2-hydroxy-2- methylpropylamino)cyclohexyl)methyl)- 3,5-bis(trifluoromethyl)benzamide | 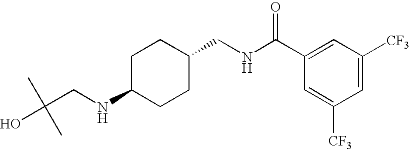 | 441.19 |
| 5 | 3-fluoro-N-((cis-4-(4-hydroxy-3,5- dimethylbenzylamino)cyclohexyl)methyl)- 5-(trifluoromethyl)benzamide | 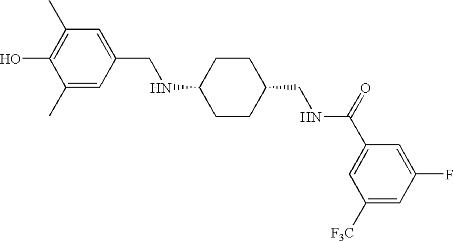 | 453.21 |
| 6 | tert-butyl 1-((cis-4-((3,5- bis(trifluoromethyl)benzamido)methyl) cyclohexylamino)methyl) cyclopentylcarbamate | 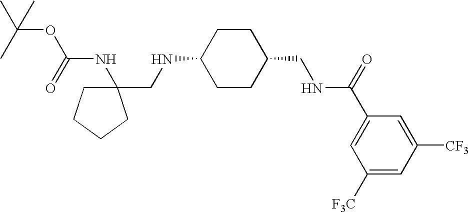 | 566.27 |
| 7 | N-((cis-4-(2- hydroxybenzylamino)cyclohexyl)methyl)- 3,5-bis(trifluoromethyl)benzamide | 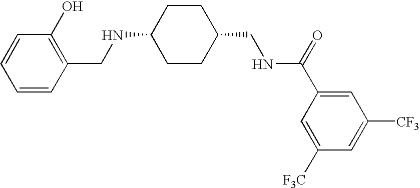 | 475.17 |
| 8 | N-((cis-4-((2-fluoropyridin-3- yl)methylamino)cyclohexyl)methyl)- 3,5-bis(trifluoromethyl)benzamide | 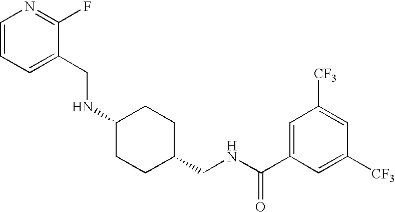 | 478.17 |
| 9 | N-((cis-4-(2-hydroxy-3- methylbenzylamino)cyclohexyl)methyl)- 3,5-bis(trifluoromethyl)benzamide | 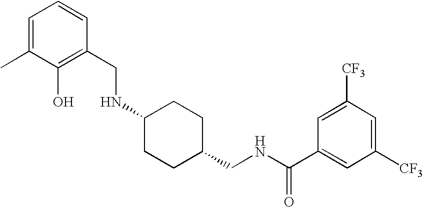 | 489.19 |
| 10 | N-((cis-4-(2- methoxybenzylamino)cyclohexyl)methyl)- 3,5-bis(trifluoromethyl)benzamide | 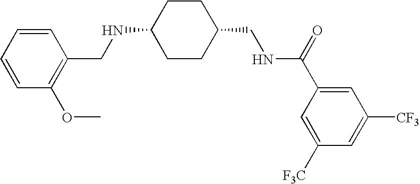 | 489.19 |
| 11 | N-((cis-4-((3-methoxypyridin-2- yl)methylamino)cyclohexyl)methyl)- 3,5-bis(trifluoromethyl)benzamide | 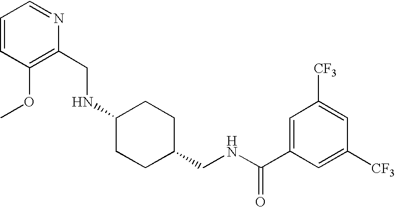 | 490.19 |
| 12 | N-((cis-4-(3-fluoro-2- hydroxybenzylamino)cyclohexyl)methyl)- 3,5-bis(trifluoromethyl)benzamide | 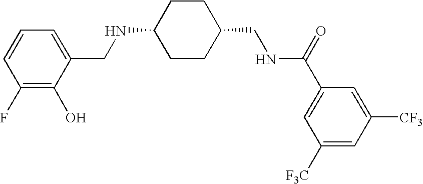 | 493.16 |
| 13 | N-((cis-4-(2-chloro-6- fluorobenzylamino)cyclohexyl)methyl)- 3,5-bis(trifluoromethyl)benzamide | 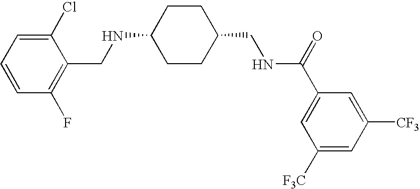 | 511.13 |
| 14 | N-((cis-4-(2,4- dimethoxybenzylamino)cyclohexyl) methyl)-3,5-bis(trifluoromethyl)benzamide | 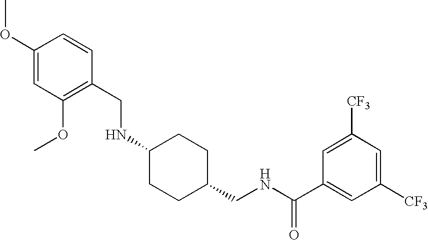 | 519.20 |
| 15 | 3-fluoro-N-((cis-4-(2- hydroxybenzylamino)cyclohexyl)methyl)- 5-(trifluoromethyl)benzamide | 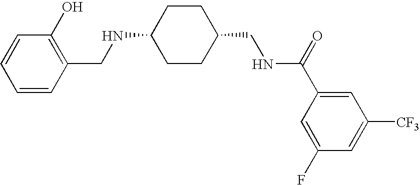 | 425.18 |
| 16 | 3-fluoro-N-((cis-4-((3-hydroxypyridin- 2-yl)methylamino)cyclohexyl)methyl)- 5-(trifluoromethyl)benzamide | 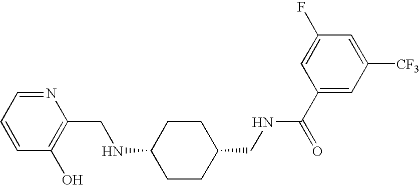 | 426.17 |
| 17 | 3-fluoro-N-((cis-4-((2-fluoropyridin-3- yl)methylamino)cyclohexyl)methyl)-5- (trifluoromethyl)benzamide | 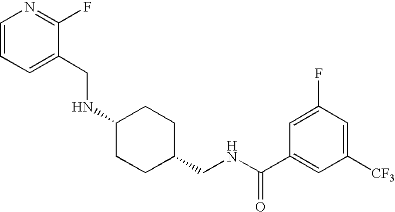 | 428.17 |
| 18 | 3-fluoro-N-((cis-4-(2-hydroxy-3- methylbenzylamino)cyclohexyl)methyl)- 5-(trifluoromethyl)benzamide | 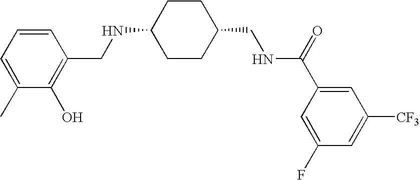 | 439.19 |
| 19 | 3-fluoro-N-((cis-4-(2- methoxybenzylamino)cyclohexyl)methyl)- 5-(trifluoromethyl)benzamide | 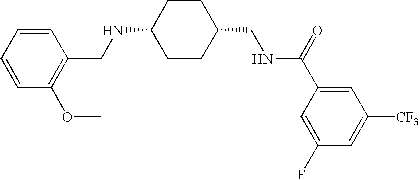 | 439.19 |
| 20 | 3-fluoro-N-((cis-4-((3-methoxypyridin- 2-yl)methylamino)cyclohexyl)methyl)- 5-(trifluoromethyl)benzamide | 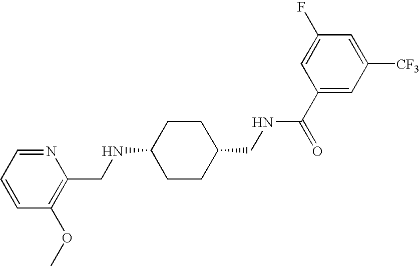 | 440.19 |
| 21 | 3-fluoro-N-((cis-4-(3-fluoro-2- hydroxybenzylamino)cyclohexyl)methyl)- 5-(trifluoromethyl)benzamide | 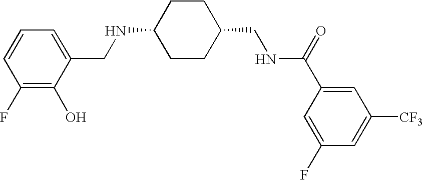 | 443.17 |
| 22 | 3-fluoro-N-((cis-4-(2-fluoro-6- hydroxybenzylamino)cyclohexyl)methyl)- 5-(trifluoromethyl)benzamide | 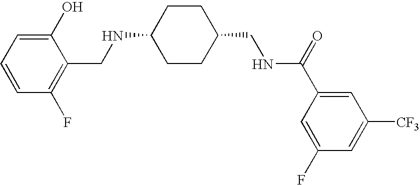 | 443.17 |
| 23 | N-((cis-4-(2-chloro-6- fluorobenzylamino)cyclohexyl)methyl)- 3-fluoro-5-(trifluoromethyl)benzamide | 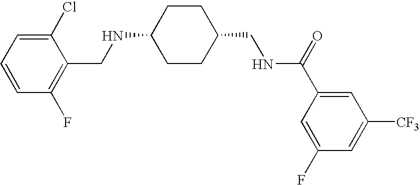 | 461 .13 |
| 24 | N-((cis-4-(2,4- dimethoxybenzylamino)cyclohexyl) methyl)-3-fluoro-5- (trifluoromethyl)benzamide | 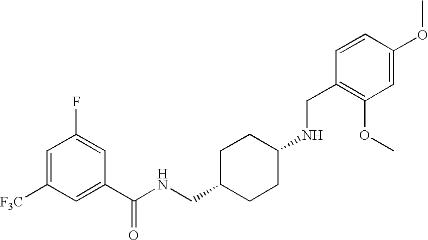 | 469.20 |
| 25 | N-((trans-4-(2- hydroxybenzylamino)cyclohexyl)methyl)- 3,5-bis(trifluoromethyl)benzamide | 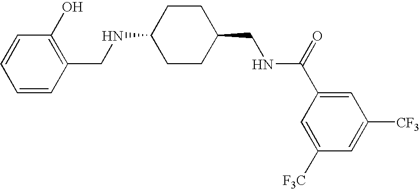 | 475.17 |
| 26 | N-((trans-4-((3-hydroxypyridin-2- yl)methylamino)cyclohexyl)methyl)- 3,5-bis(trifluoromethyl)benzamide | 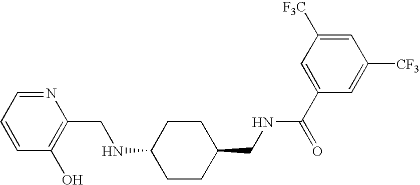 | 476.17 |
| 27 | N-((trans-4-((2-fluoropyridin-3- yl)methylamino)cyclohexyl)methyl)- 3,5-bis(trifluoromethyl)benzamide | 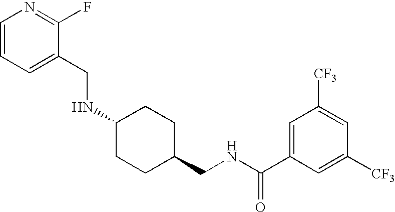 | 478.17 |
| 28 | N-((trans-4-(2-hydroxy-3- methylbenzylamino)cyclohexyl)methyl)- 3,5-bis(trifluoromethyl)benzamide | 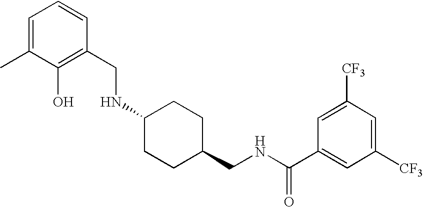 | 489.19 |
| 29 | N-((trans-4-(2- methoxybenzylamino)cyclohexyl)methyl)- 3,5-bis(trifluoromethyl)benzamide | 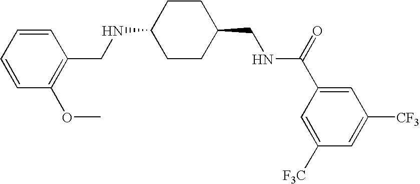 | 489.19 |
| 30 | N-((trans-4-((3-methoxypyridin-2- yl)methylamino)cyclohexyl)methyl)- 3,5-bis(trifluoromethyl)benzamide | 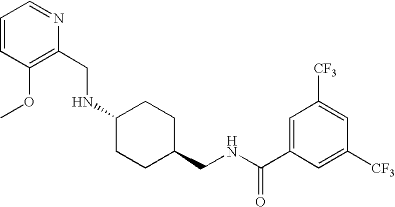 | 490.19 |
| 31 | N-((trans-4-(3-fluoro-2- hydroxybenzylamino)cyclohexyl)methyl)- 3,5-bis(trifluoromethyl)benzamide | 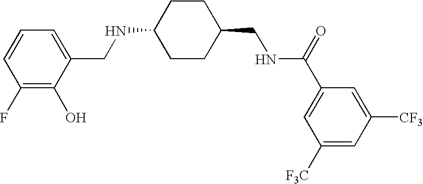 | 493.16 |
| 32 | N-((trans-4-(2-fluoro-6- hydroxybenzylamino)cyclohexyl)methyl)- 3,5-bis(trifluoromethyl)benzamide | 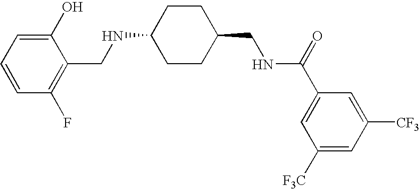 | 493.16 |
| 33 | N-((trans-4-(2-hydroxy-6- methoxybenzylamino)cyclohexyl)methyl)- 3,5-bis(trifluoromethyl)benzamide | 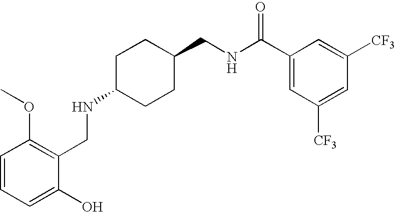 | 505.18 |
| 34 | N-((trans-4-(2-chloro-6- fluorobenzylamino)cyclohexyl)methyl)- 3,5-bis(trifluoromethyl)benzamide | 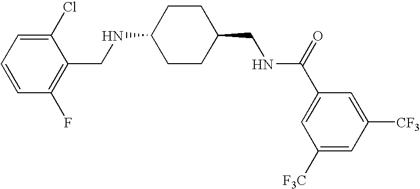 | 511.13 |
| 35 | N-((trans-4-(2,4- dimethoxybenzylamino)cyclohexyl) methyl)-3,5-bis(trifluoromethyl)benzamide | 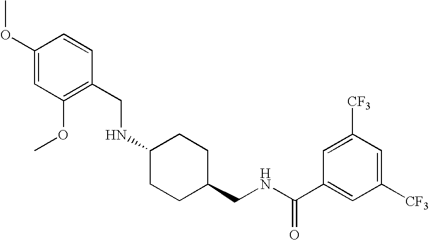 | 519.20 |
| 36 | 2-(cis-4-(2-hydroxy-4- methoxybenzylamino)cyclohexyl)-N-(4- p-tolylthiazol-2-yl)acetamide | 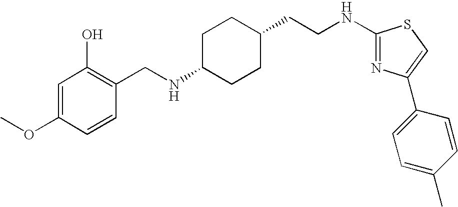 | 466.21 |
| 37 | tert-butyl 1-(cis-4-(2-oxo-2-(4-p- tolylthiazol-2- ylamino)ethyl)cyclohexylamino)methyl) cyclopentylcarbamate | 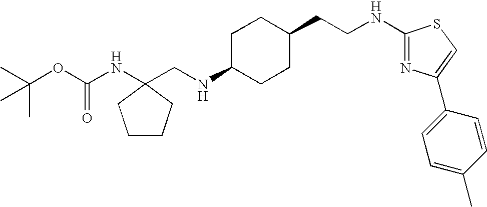 | 527.30 |
| 38 | tert-butyl 2-methyl-1-(cis-4-(2-oxo-2-(4- p-tolylthiazol-2- ylamino)ethyl)cyclohexylamino)propan- 2-ylcarbamate | 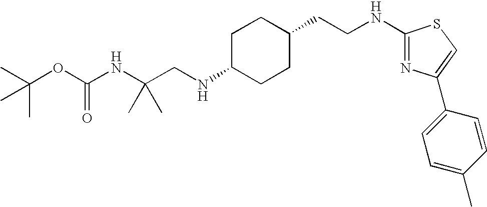 | 501.28 |
| 39 | N-((cis-4-(2-oxo-2-(pyrrolidin-1- yl)ethylamino)cyclohexyl)methyl)-3,5- bis(trifluoromethyl)benzamide | 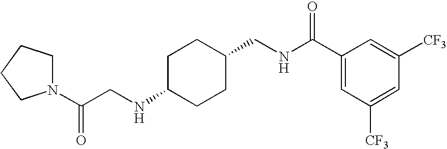 | 480.20 |
| 40 | N-((cis-4-(2-(tert-butylamino)-2- oxoethylamino)cyclohexyl)methyl)-3,5- bis(trifluoromethyl)benzamide | 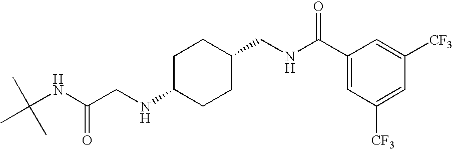 | 482.22 |
| 41 | N-((cis-4-(2-(diethylamino)-2- oxoethylamino)cyclohexyl)methyl)-3,5- bis(trifluoromethyl)benzamide | 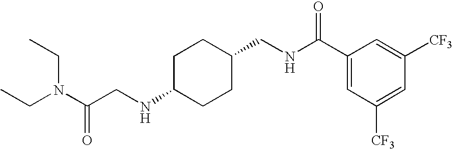 | 482.22 |
| 42 | N-((cis-4-(2-oxo-2-(pentan-3- ylamino)ethylamino)cyclohexyl)methyl)- 3,5-bis(trifluoromethyl)benzamide | 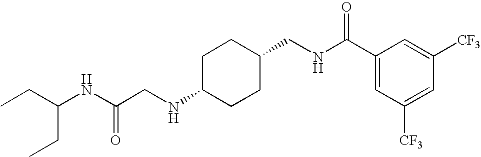 | 496.23 |
| 43 | N-((cis-4-(2-oxo-2-(2,2,2- trifluoroethylamino)ethylamino) cyclohexyl)methyl)-3,5- bis(trifluoromethyl)benzamide | 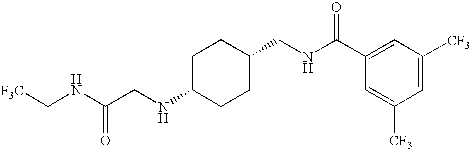 | 508.16 |
| 44 | N-((cis-4-(2- (cyclopentyl(methyl)amino)-2- oxoethylamino)cyclohexyl)methyl)-3,5- bis(trifluoromethyl)benzamide | 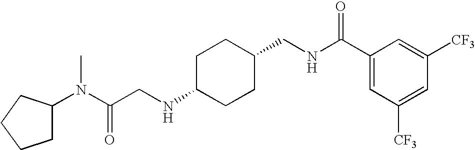 | 508.23 |
| 45 | N-((cis-4-(2-oxo-2-(piperidin-1- ylamino)ethylamino)cyclohexyl)methyl)- 3,5-bis(trifluoromethyl)benzamide | 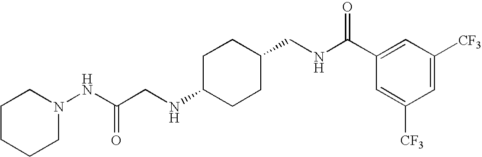 | 509.23 |
| 46 | N-((cis-4-(2-(3,3-difluoropyrrolidin-1- yl)-2- oxoethylamino)cyclohexyl)methyl)-3,5- bis(trifluoromethyl)benzamide | 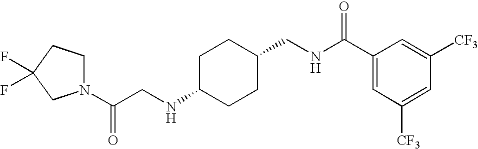 | 516.18 |
| 47 | N-((cis-4-(2-(3-ethylpent-1-yn-3- ylamino)-2- oxoethylamino)cyclohexyl)methyl)-3,5- bis(trifluoromethyl)benzamide | 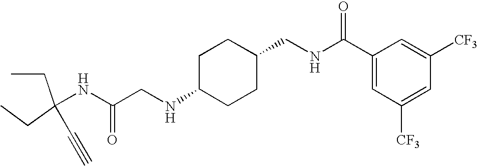 | 520.23 |
| 48 | N-((cis-4-(2-(cyclohexylmethylamino)- 2-oxoethylamine)cyclohexyl)methyl)- 3,5-bis(trifluoromethyl)benzamide | 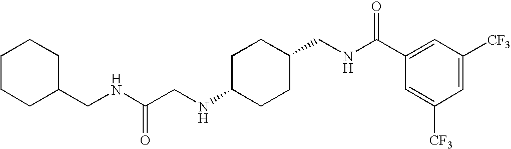 | 522.25 |
| 49 | N-((cis-4-(2- (cyclohexyl(methyl)amino)-2- oxoethylamino)cyclohexyl)methyl)-3,5- bis(trifluoromethyl)benzamide | 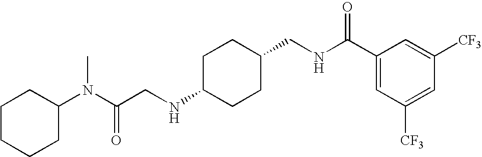 | 522.25 |
| 50 | N-((cis-4-(2-((4- methoxycyclohexyl)(methyl)amino)-2- oxoethylamino)cyclohexyl)methyl)-3,5- bis(trifluoromethyl)benzamide | 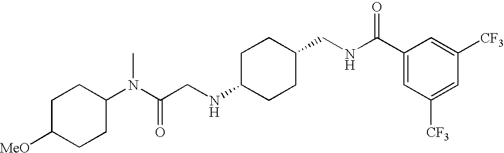 | 552.26 |
| 51 | N-((cis-4-((5-tert-butyl-1,2,4-oxadiazol- 3-yl)methylamino)cyclohexyl)methyl)- 3,5-bis(trifluoromethyl)benzamide | 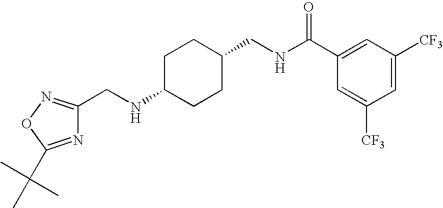 | 507.21 |
| 52 | N-((cis-4-((5-methylisoxazol-3- yl)methylamino)cyclohexyl)methyl)- 3,5-bis(trifluoromethyl)benzamide | 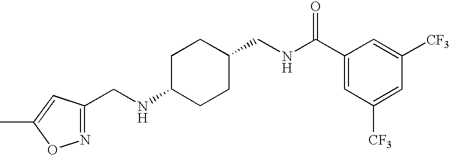 | 464.17 |
| 53 | N-((cis-4-((2-methylthiazol-4- yl)methylamino)cyclohexyl)methyl)- 3,5-bis(trifluoromethyl)benzamide | 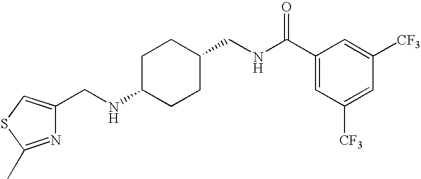 | 480.15 |
| 54 | N-((cis-4-(2-(2-ethylpyrrolidin-1-yl)-2- oxoethylamino)cyclohexyl)methyl)-3,5- bis(trifluoromethyl)benzamide | 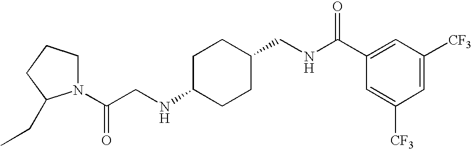 | 508.23 |
| 55 | N-((trans-4-(2-(tert-butylamino)-2- oxoethylamino)cyclohexyl)methyl)-3,5- bis(trifluoromethyl)benzamide | 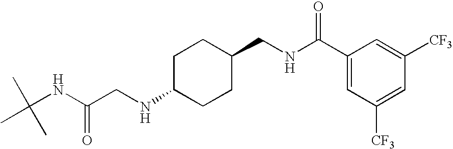 | 482.22 |
| 56 | N-((trans-4-(2-(diethylamino)-2- oxoethylamino)cyclohexyl)methyl)-3,5- bis(trifluoromethyl)benzamide | 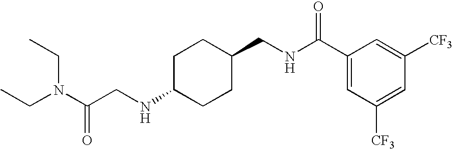 | 482.22 |
| 57 | N-((trans-4-(2-oxo-2-(pentan-3- ylamino)ethylamino)cyclohexyl)methyl)- 3,5-bis(trifluoromethyl)benzamide | 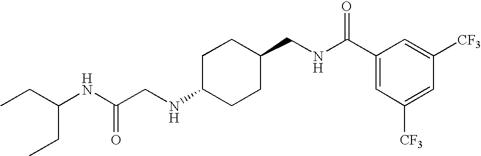 | 496.23 |
| 58 | N-((trans-4-(2-oxo-2-(2,2,2- trifluoroethylamino)ethylamino) cyclohexyl)methyl)-3,5- bis(trifluoromethyl)benzamide | 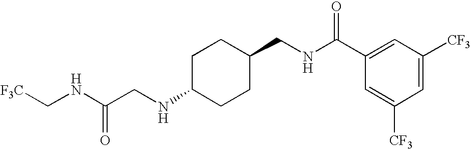 | 508.16 |
| 59 | N-((trans-4-(2- (cyclopentyl(methyl)amino)-2- oxoethylamino)cyclohexyl)methyl)-3,5- bis(trifluoromethyl)benzamide | 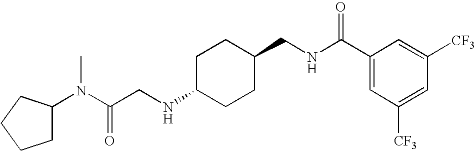 | 508.23 |
| 60 | N-((trans-4-(2-oxo-2-(piperidin-1- ylamino)ethylamino)cyclohexyl)methyl)- 3,5-bis(trifluoromethyl)benzamide | 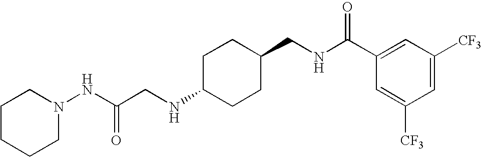 | 509.23 |
| 61 | N-((trans-4-(2-(3,3-difluoropyrrolidin-1- yl)-2- oxoethylamino)cyclohexyl)methyl)-3,5- bis(trifluoromethyl)benzamide | 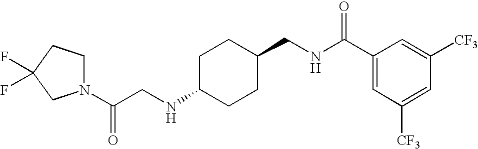 | 516.18 |
| 62 | N-((trans-4-(2-(3-ethylpent-1-yn-3- ylamino)-2- oxoethylamino)cyclohexyl)methyl)-3,5- bis(trifluoromethyl)benzamide | 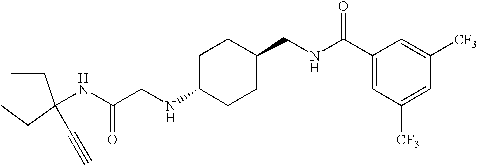 | 520.23 |
| 63 | N-((trans-4-(2- (cyclohexylmethylamino)-2- oxoethylamino)cyclohexyl)methyl)-3,5- bis(trifluoromethyl)benzamide | 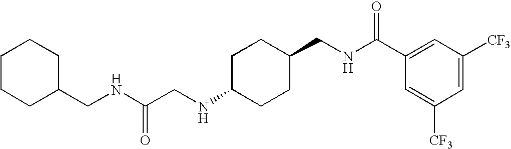 | 522.25 |
| 64 | N-((trans-4-(2- (cyclohexyl(methyl)amino)-2- oxoethylamino)cyclohexyl)methyl)-3,5- bis(trifluoromethyl)benzamide | 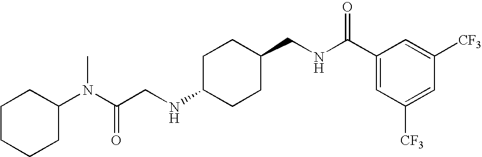 | 522.25 |
| 65 | N-((trans-4-(2-((4- methoxycyclohexyl)(methyl)amino)-2- oxoethylamino)cyclohexyl)methyl)-3,5- bis(trifluoromethyl)benzamide | 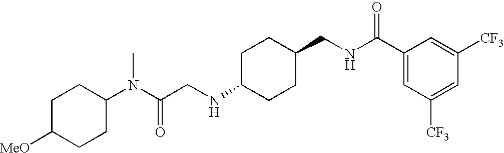 | 552.26 |
| 66 | N-((trans-4-((5-tert-butyl-1,2,4- oxadiazol-3- yl)methylamino)cyclohexyl)methyl)- 3,5-bis(trifluoromethyl)benzamide | 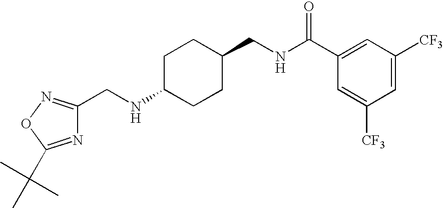 | 507.21 |
| 67 | N-((trans-4-((5-methylisoxazol-3- yl)methylamino)cyclohexyl)methyl)- 3,5-bis(trifluoromethyl)benzamide | 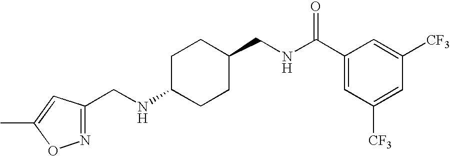 | 464.17 |
| 68 | N-((trans-4-(2-(2-ethylpyrrolidin-1-yl)- 2-oxoethylamino)cyclohexyl)methyl)- 3,5-bis(trifluoromethyl)benzamide | 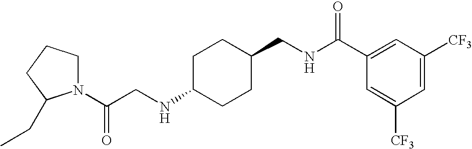 | 508.23 |
| 69 | N-((cis-4-(2-(isopropylamino)-2- oxoethylamino)cyclohexyl)methyl)-3,5- bis(trifluoromethyl)benzamide | 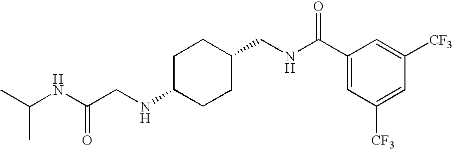 | 468.20 |
| 70 | N-((cis-4-(2-(isobutylamino)-2- oxoethylamino)cyclohexyl)methyl)-3,5- bis(trifluoromethyl)benzamide | 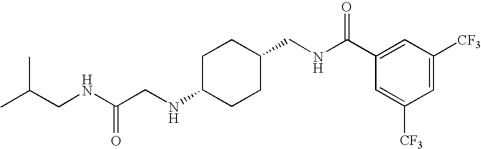 | 482.22 |
| 71 | N-((cis-4-(2-(sec-butylamino)-2- oxoethylamino)cyclohexyl)methyl)-3,5- bis(trifluoromethyl)benzamide | 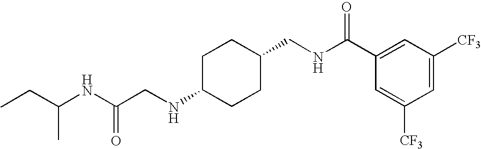 | 482.22 |
| 72 | N-((cis-4-(2-(2-methylpiperidin-1-yl)-2- oxoethylamino)cyclohexyl)methyl)-3,5- bis(trifluoromethyl)benzamide | 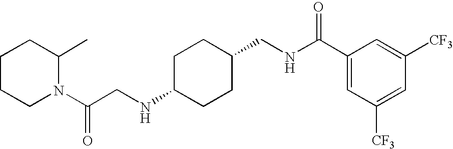 | 508.23 |
| 73 | N-((cis-4-(2-(5-methylpyridin-2- ylamino)-2- oxoethylamino)cyclohexyl)methyl)-3,5- bis(trifluoromethyl)benzamide | 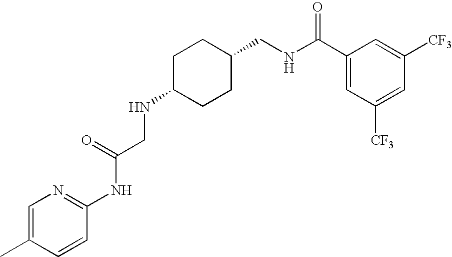 | 517.20 |
| 74 | N-((trans-4-(2-(isopropylamino)-2- oxoethylamino)cyclohexyl)methyl)-3,5- bis(trifluoromethyl)benzamide | 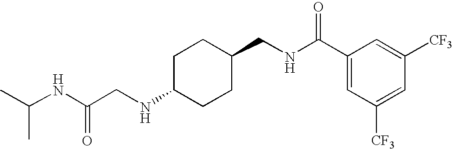 | 468.20 |
| 75 | N-((trans-4-(2-(isobutylamino)-2- oxoethylamino)cyclohexyl)methyl)-3,5- bis(trifluoromethyl)benzamide | 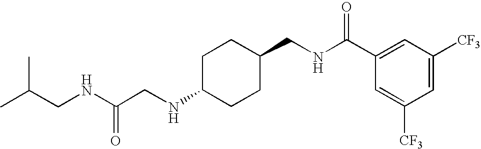 | 482.22 |
| 76 | N-((trans-4-(2-(2-methylpiperidin-1-yl)- 2-oxoethylamino)cyclohexyl)methyl)- 3,5-bis(trifluoromethyl)benzamide | 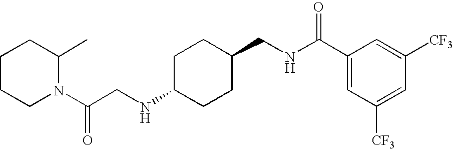 | 508.23 |
| 77 | N-((trans-4-(2-(5-methylpyridin-2- ylamino)-2- oxoethylamino)cyclohexyl)methyl)-3,5- bis(trifluoromethyl)benzamide | 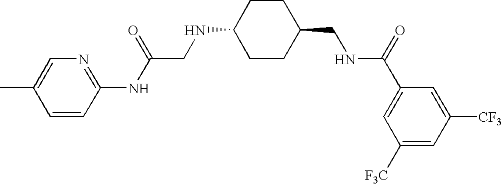 | 517.20 |
| 78 | N-(trans-4-((2-(tert-butylamino)-2- oxoethylamino)methyl)cyclohexyl)-3,5- bis(trifluoromethyl)benzamide | 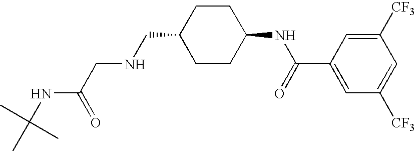 | 482.22 |
| 79 | N-(trans-4-((2-(diethylamino)-2- oxoethylamino)methyl)cyclohexyl)-3,5- bis(trifluoromethyl)benzamide | 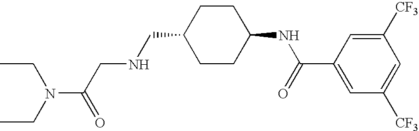 | 482.22 |
| 80 | N-(trans-4-((2-morpholino-2- oxoethylamino)methyl)cyclohexyl)-3,5- bis(trifluoromethyl)benzamide | 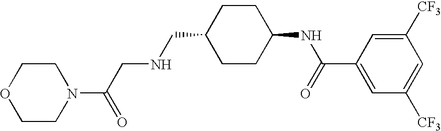 | 496.20 |
| 81 | N-(trans-4-((2-oxo-2-(pentan-3- ylamino)ethylamino)methyl)cyclohexyl)- 3,5-bis(trifluoromethyl)benzamide | 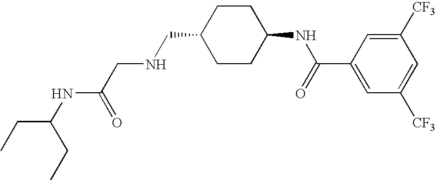 | 496.23 |
| 82 | N-(trans-4-((2-oxo-2-(2,2,2- trifluoroethylamino)ethylamino)methyl) cyclohexyl)-3,5- bis(trifluoromethyl)benzamide | 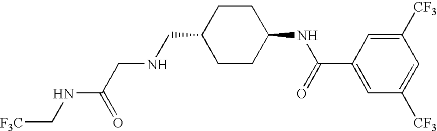 | 508.16 |
| 83 | N-(trans-4-((2- (cyclopentyl(methyl)amino)-2- oxoethylamino)methyl)cyclohexyl)-3,5- bis(trifluoromethyl)benzamide | 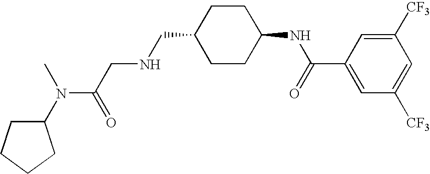 | 508.23 |
| 84 | N-(trans-4-((2-oxo-2-(piperidin-1- ylamino)ethylamino)methyl)cyclohexyl)- 3,5-bis(trifluoromethyl)benzamide | 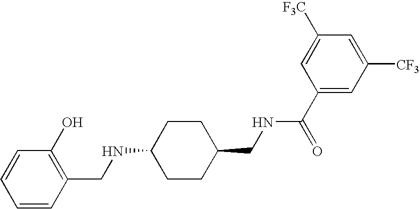 | 509.23 |
| 85 | N-(trans-4-((2-(3-ethylpent-1-yn-3- ylamino)-2- oxoethylamino)methyl)cyclohexyl)-3,5- bis(trifluoromethyl)benzamide | 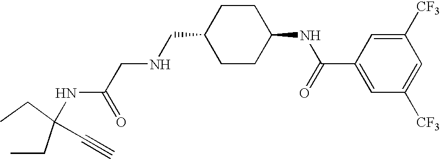 | 520.23 |
| 86 | N-(trans-4-((2- (cyclohexylmethylamino)-2- oxoethylamino)methyl)cyclohexyl)-3,5- bis(trifluoromethyl)benzamide | 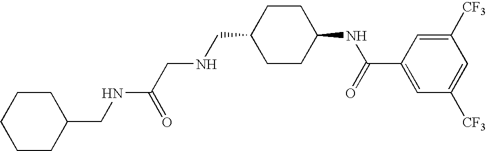 | 522.25 |
| 87 | N-(trans-4-((2- (cyclohexyl(methyl)amino)-2- oxoethylamino)methyl)cyclohexyl)-3,5- bis(trifluoromethyl)benzamide | 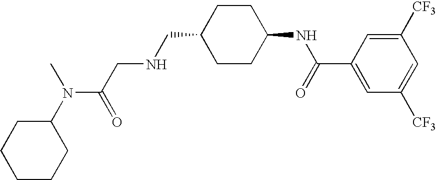 | 522.25 |
| 88 | N-(trans-4-((2-((4- methoxycyclohexyl)(methyl)amino)-2- oxoethylamino)methyl)cyclohexyl)-3,5- bis(trifluoromethyl)benzamide | 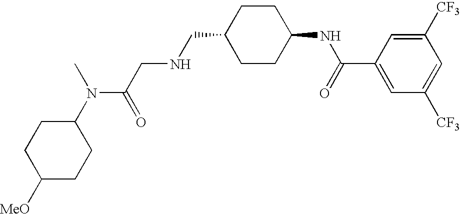 | 552.26 |
| 89 | N-(trans-4-(((5-tert-butyl-1,2,4- oxadiazol-3- yl)methylamino)methyl)cyclohexyl)- 3,5-bis(trifluoromethyl)benzamide | 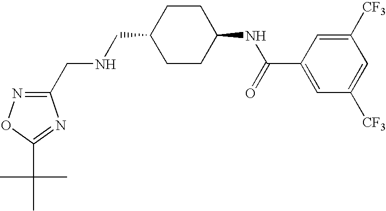 | 507.21 |
| 90 | N-(trans-4-(((5-methylisoxazol-3- yl)methylamino)methyl)cyclohexyl)- 3,5-bis(trifluoromethyl)benzamide | 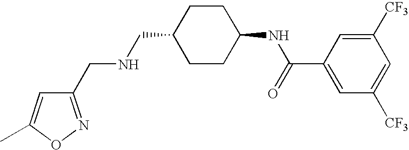 | 464.17 |
| 91 | N-(trans-4-((2-(2-ethylpyrrolidin-1-yl)- 2-oxoethylamino)methyl)cyclohexyl)- 3,5-bis(trifluoromethyl)benzamide | 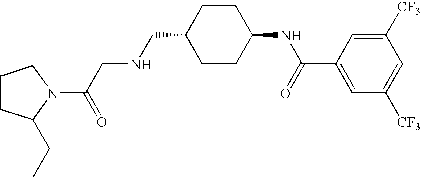 | 508.23 |
| 92 | N-(trans-4-((2-(isopropylamino)-2- oxoethylamino)methyl)cyclohexyl)-3,5- bis(trifluoromethyl)benzamide | 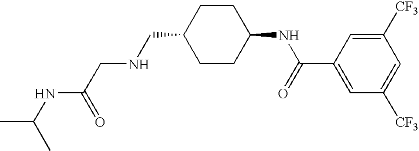 | 468.20 |
| 93 | N-(trans-4-((2-(isobutylamino)-2- oxoethylamino)methyl)cyclohexyl)-3,5- bis(trifluoromethyl)benzamide | 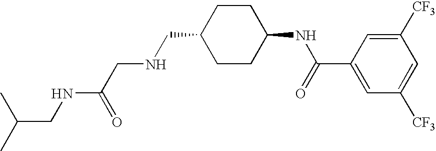 | 482.22 |
| 94 | N-(trans-4-((2-(sec-butylamino)-2- oxoethylamino)methyl)cyclohexyl)-3,5- bis(trifluoromethyl)benzamide | 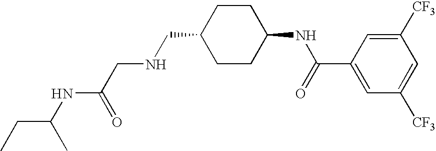 | 482.22 |
| 95 | N-(trans-4-((2-(2-methylpiperidin-1-yl)- 2-oxoethylamino)methyl)cyclohexyl)- 3,5-bis(trifluoromethyl)benzamide | 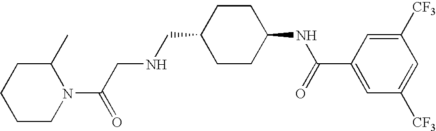 | 508.23 |
| 96 | N-(trans-4-((2-oxo-2-(pyridin-2- ylamino)ethylamino)methyl)cyclohexyl)- 3,5-bis(trifluoromethyl)benzamide | 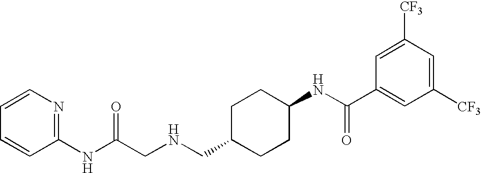 | 517.20 |
| 97 | tert-butyl 2-(cis-4-((3-fluoro-5- (trifluoromethyl)benzamido)methyl) cyclohexylamino)ethylcarbamate | 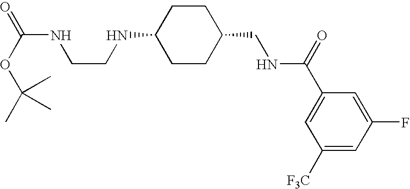 | 462.23 |
| 98 | tert-butyl 2-(cis-4-((3,5- bis(trifluoromethyl)benzamido)methyl) cyclohexylamino)ethylcarbamate | 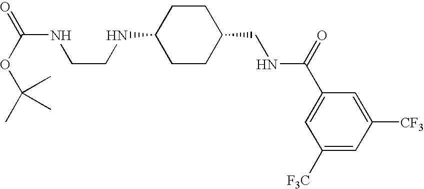 | 512.23 |
| 99 | tert-butyl 2-(cis-4-((3-chloro-5- (trifluoromethyl)benzamido)methyl) cyclohexylamino)ethylcarbamate | 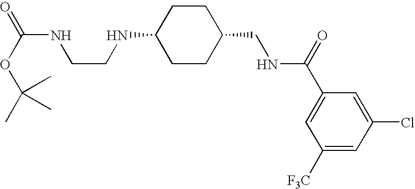 | 478.20 |
| 100 | tert-butyl 2-(cis-4-((3-methoxy-5- (trifluoromethyl)benzamido)methyl) cyclohexylamino)ethylcarbamate | 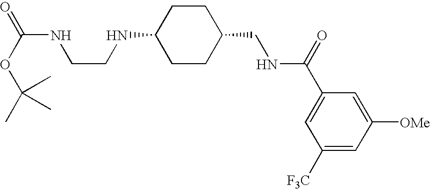 | 474.25 |
| 101 | N-((trans-4-(2-(3,3- dimethylbutanamido)ethylamino) cyclohexyl)methyl)-3,5- bis(trifluoromethyl)benzamide | 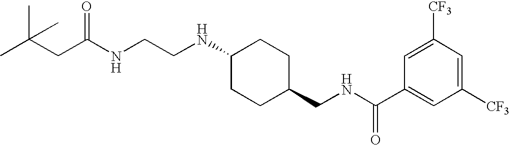 | 510.25 |
| 102 | N-((trans-4-(2-(4,4,4- trifluorobutanamido)ethylamino) cyclohexyl)methyl)-3,5- bis(trifluoromethyl)benzamide | 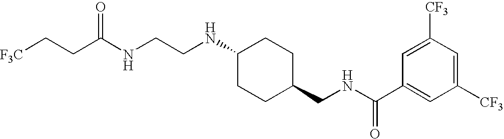 | 536.19 |
| 103 | N-((trans-4-(2-(2- cyclopropylacetamido)ethylamino) cyclohexyl)methyl)-3,5- bis(trifluoromethyl)benzamide | 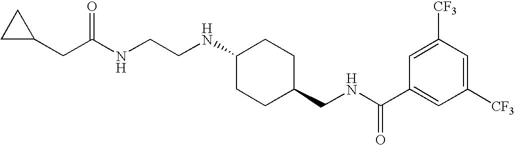 | 494.22 |
| 104 | N-((trans-4-(2- pivalamidoethylamino)cyclohexyl) methyl)-3,5-bis(trifluoromethyl)benzamide | 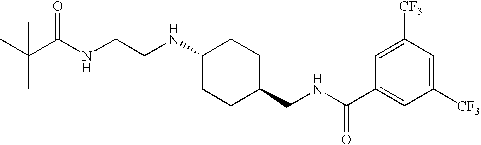 | 496.23 |
| 105 | tert-butyl cis-4-(2-(3,5- bis(trifluoromethyl)phenylamino)-2- oxoethyl)cyclohexylcarbamate | 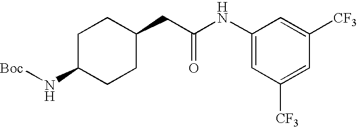 | 469.18 |
| 106 | 2-(cis-4-aminocyclohexyl)-N-(3,5- bis(trifluoromethyl)phenyl)acetamide | 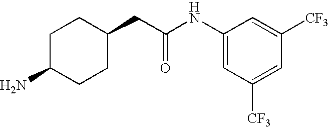 | 369.13 |
| 107 | tert-butyl trans-4-(2-oxo-2-(4-p- tolylthiazol-2- ylamino)ethyl)cyclohexylcarbamate | 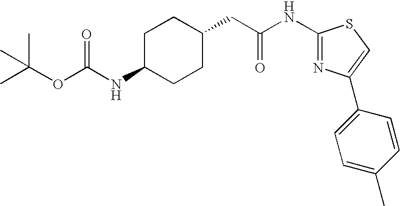 | 430.21 |
| 108 | (S)-N-((trans)-4-(2-(3,5- bis(trifluoromethyl)phenylamino)-2- oxoethyl)cyclohexyl)-2-hydroxy-3,3- dimethylbutanamide | 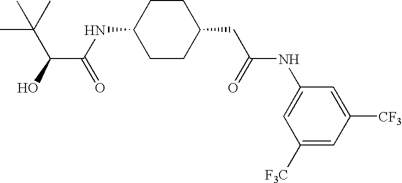 | 483.20 |
| 109 | 1-amino-N-(cis-4-(2-oxo-2-(3- (trifluoromethoxy)benzylamino)ethyl) cyclohexyl)cyclohexanecarboxamide | 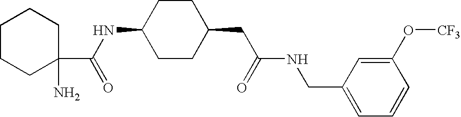 | 456.24 |
| 110 | (R)-N-(cis-4-(2-oxo-2-(3- (trifluoromethoxy)benzylamino)ethyl) cyclohexyl)pyrrolidine-2-carboxamide | 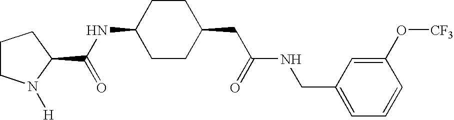 | 428.21 |
| 111 | (S)-N-((cis-4-(2-oxo-2-(3- (trifluoromethoxy)benzylamino)ethyl) cyclohexyl)pyrrolidine-2-carboxamide | 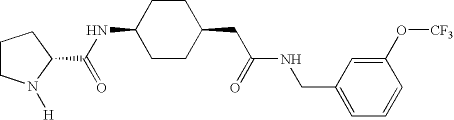 | 428.21 |
| 112 | (1R,2R)-2-amino-N-(cis-4-(2-oxo-2-(3- (trifluoromethoxy)benzylamino)ethyl) cyclohexyl)cyclohexanecarboxamide | 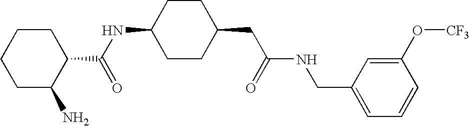 | 456.24 |
| 113 | (S)-N-(cis-4-(2-oxo-2-(3- (trifluoromethoxy)benzylamino)ethyl) cyclohexyl)piperidine-2-carboxamide | 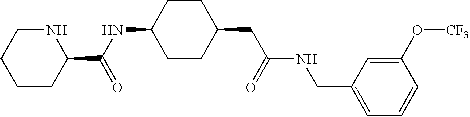 | 442.22 |
| 114 | N-(cis-4-(2-oxo-2-(3- (trifluoromethoxy)benzylamino)ethyl) cyclohexyl)-4-phenylpiperidin-4- carboxamide | 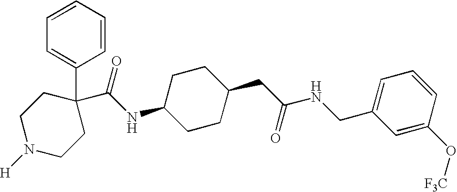 | 518.26 |
| 115 | (1R,2S)-2-amino-N-(cis-4-(2-oxo-2-(3- (trifluoromethoxy)benzylamino)ethyl) cyclohexyl)cyclohexanecarboxamide | 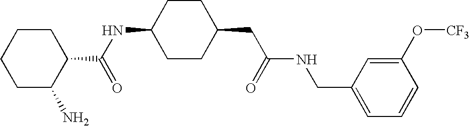 | 456.24 |
| 116 | (2S,4S)-N-(cis-4-(2-oxo-2-(3- (trifluoromethoxy)benzylamino)ethyl) cyclohexyl)-4-phenylpyrrolidine-2- carboxamide | 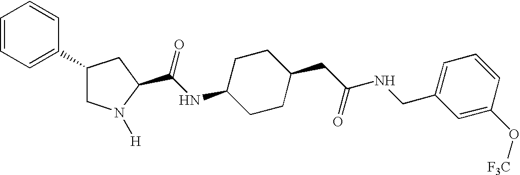 | 504.24 |
| 117 | 1-amino-N-(trans-4-(2-(3,5- bis(trifluoromethyl)phenylamino)-2- oxoethyl)cyclohexyl) cyclohexanecarboxamide | 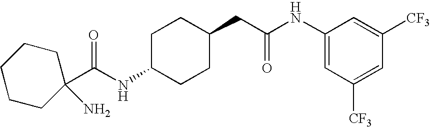 | 494.22 |
| 118 | (R)-N-(trans-4-(2-(3,5- bis(trifluoromethyl)phenylamino)-2- oxoethyl)cyclohexyl)pyrrolidine-2- carboxamide | 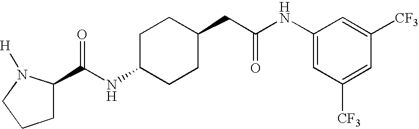 | 466.19 |
| 119 | (S)-N-(trans-4-(2-(3,5- bis(trifluoromethyl)phenylamino)-2- oxoethyl)cyclohexyl)pyrrolidine-2- carboxamide | 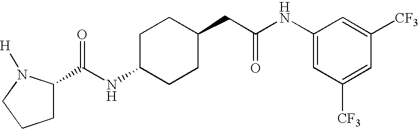 | 466.19 |
| 120 | (S)-N-(trans-4-(2-(3,5- bis(trifluoromethyl)phenylamino)-2- oxoethyl)cyclohexyl)piperidine-2- carboxamide | 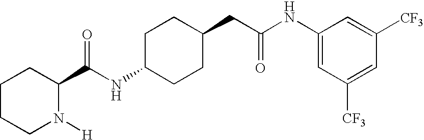 | 480.20 |
| 121 | (S)-N-(trans-4-(2-(3,5- bis(trifluoromethyl)phenylamino)-2- oxoethyl)cyclohexyl)-4- phenylpiperidine-4-carboxamide | 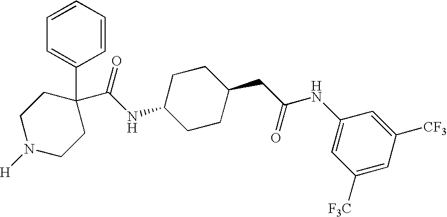 | 556.23 |
| 122 | (1R,2S)-2-amino-N-(trans-4-(2-(3,5- bis(trifluoromethyl)phenylamino)-2- oxoethyl)cyclohexyl) cyclohexanecarboxamide | 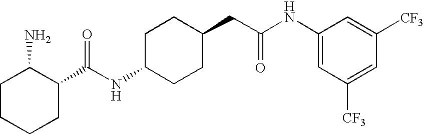 | 494.22 |
| 123 | (2S,4S)-N-(trans-4-(2-(3,5- bis(trifluoromethyl)phenylamino)-2- oxoethyl)cyclohexyl)-4- phenylpyrrolidine-2-carboxamide | 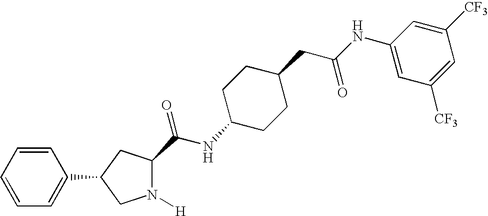 | 542.22 |
| 124 | 1-amino-N-(trans-4-(2-(3,5- bis(trifluoromethyl)phenylamino)-2- oxoethyl)cyclohexyl) cyclopentanecarboxamide | 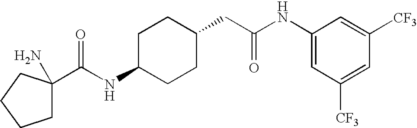 | 480.20 |
| 125 | (2S,4S)-N-(cis-4-((3,5- bis(trifluoromethyl)benzamido)methyl) cyclohexyl)-4-phenylpyrrolidine-2- carboxamide | 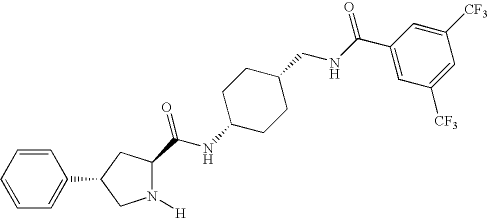 | 542.22 |
| 126 | (2S,4R)-N-(cis-4-((3,5- bis(trifluoromethyl)benzamido)methyl) cyclohexyl)-4-phenylpyrrolidine-2- carboxamide | 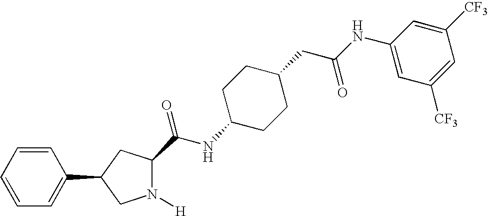 | 542.22 |
| 127 | (2S,5R)-N-(cis-4-((3,5- bis(trifluoromethyl)benzamido)methyl) cyclohexyl)-5-phenylpyrrolidine-2- carboxamide | 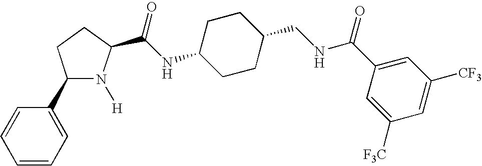 | 542.22 |
| 128 | N-(cis-4-((3,5- bis(trifluoromethyl)benzamido)methyl) cyclohexyl)-4-phenylpiperidine-4- carboxamide | 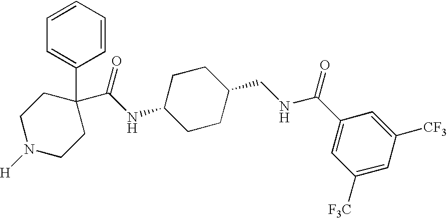 | 556.23 |
| 129 | (2S,4S)-N-(cis-4-(2-oxo-2-(4-p- tolylthiazol-2- ylamino)ethyl)cyclohexyl)-4- phenylpyrrolidine-2-carboxamide | 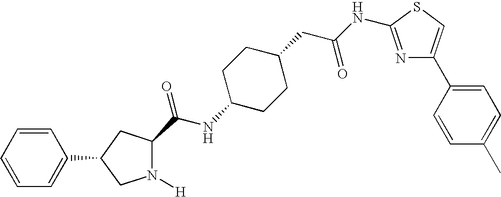 | 503.24 |
| 130 | (2S,4R)-N-(cis-4-(2-oxo-2-(4-p- tolylthiazol-2- ylamino)ethyl)cyclohexyl)-4- phenylpyrrolidine-2-carboxamide | 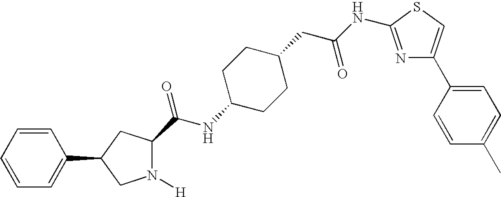 | 503.24 |
| 131 | (2S,5R)-N-(cis-4-(2-oxo-2-(4-p- tolylthiazol-2- ylamino)ethyl)cyclohexyl)-4- phenylpyrrolidine-2-carboxamide | 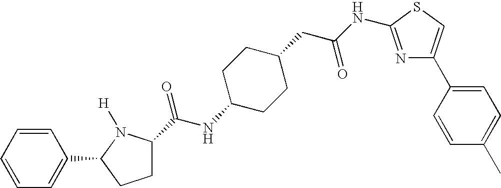 | 503.24 |
| 132 | N-(cis-4-(2-oxo-2-(4-p-tolylthiazol-2- ylamino)ethyl)cyclohexyl)-4- phenylpiperidine-4-carboxamide | 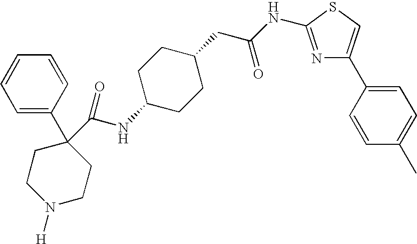 | 517.26 |
| 133 | (2S,4S)-N-(trans-4-((3,5- bis(trifluoromethyl)benzamido)methyl) cyclohexyl)-4-phenylpyrrolidine-2- carboxamide | 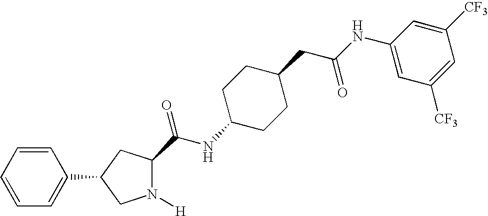 | 542.22 |
| 134 | (2S,4R)-N-(trans-4-((3,5- bis(trifluoromethyl)benzamido)methyl) cyclohexyl)-4-phenylpyrrolidine-2- carboxamide | 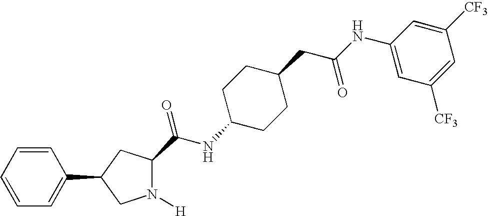 | 542.22 |
| 135 | (2S,5R)-N-(trans-4-((3,5- bis(trifluoromethyl)benzamido)methyl) cyclohexyl)-5-phenylpyrrolidine-2- carboxamide | 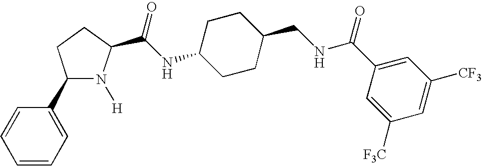 | 542.22 |
| 136 | N-(trans-4-((3,5- bis(trifluoromethyl)benzamido)methyl) cyclohexyl)-4-phenylpiperidine-4- carboxamide | 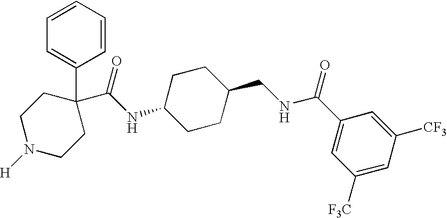 | 556.23 |
| 137 | (2R,5S)-N-(trans-4-((3,5- bis(trifluoromethyl)benzamido)methyl) cyclohexyl)-5-phenylpyrrolidine-2- carboxamide | 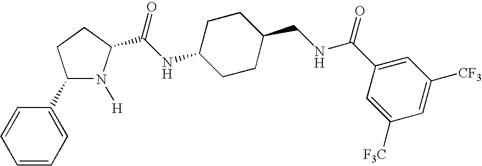 | 542.22 |
| 138 | (2R,5S)-N-(cis-4-(2-oxo-2-(4-p- tolylthiazol-2- ylamino)ethyl)cyclohexyl)-5- phenylpyrrolidine-2-carboxamide | 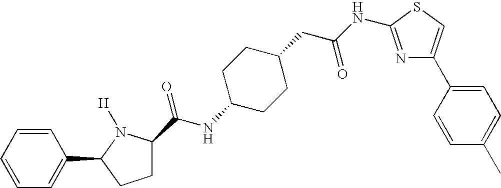 | 503.24 |
| 139 | (2R,5S)-N-(cis-4-((3,5- bis(trifluoromethyl)benzamido)methyl) cyclohexyl)-5-phenylpyrrolidine-2- carboxamide | 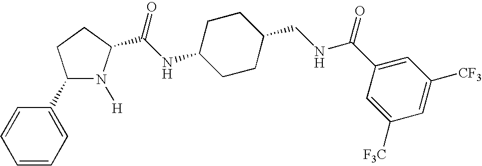 | 542.22 |
| 140 | N-((cis-4-(1- aminocyclopentanecarboxamido) cyclohexyl)methyl)-3,5- bis(trifluoromethyl)benzamide | 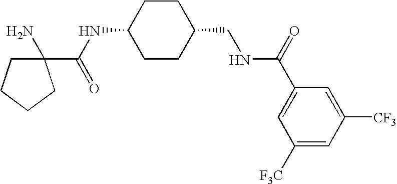 | 480.20 |
| 141 | N-((cis-4-(1- aminocyclopentanecarboxamido) cyclohexyl)methyl)-3-fluoro-5- (trifluoromethyl)benzamide | 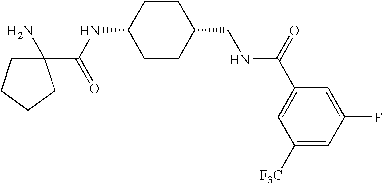 | 430.20 |
| 142 | N-((trans-4-(1- aminocyclopentanecarboxamido) cyclohexyl)methyl)-3,5- bis(trifluoromethyl)benzamide | 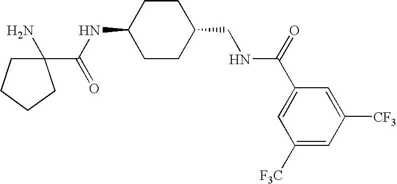 | 480.20 |
| 143 | (S)-N-(cis-4-(2-(3,5- bis(trifluoromethyl)phenylamino)-2- oxoethyl)cyclohexyl)pyrrolidine-2- carboxamide | 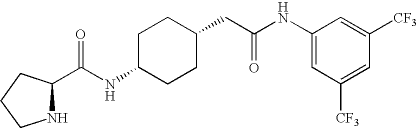 | 466.19 |
| 144 | N-(cis-4-(2-(3,5- bis(trifluoromethyl)phenylamino)-2- oxoethyl)cyclohexyl)piperidine-4- carboxamide | 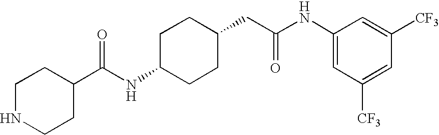 | 480.20 |
| 145 | (2R,4S)-N-(cis-4-(2-3,5-p- bis(trifluoromethyl)phenylamino)-2- oxoethyl)cyclohexyl)-4- hydroxypyrrolidine-2-carboxamide | 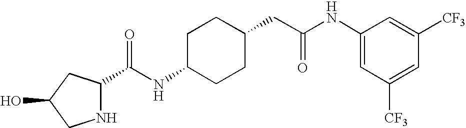 | 482.18 |
| 146 | (R)-N-(cis-4-(2-(3,5- bis(trifluoromethyl)phenylamino)-2- oxoethyl)cyclohexyl)pyrrolidine-2- carboxamide | 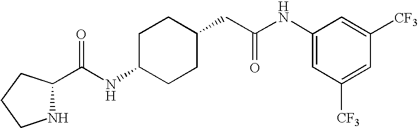 | 466.19 |
| 147 | 4-amino-N-(cis-4-(2-(3,5- bis(trifluoromethyl)phenylamino)-2- oxoethyl)cyclohexyl)tetrahydro-2H- pyran-4-carboxamide | 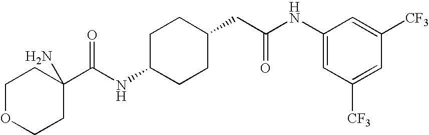 | 496.20 |
| 148 | N-(cis-4-(2-(3,5- bis(trifluoromethyl)phenylamino)-2- oxoethyl)cyclohexyl)-4- hydroxypipendine-4-carboxamide | 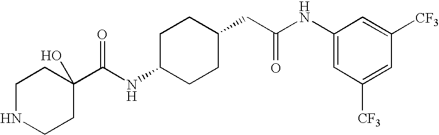 | 496.20 |
| 149 | 1-amino-N-(cis-4-(2-(3,5- bis(trifluoromethyl)phenylamino)-2- oxoethyl)cyclohexyl) cyclopropanecarboxamide | 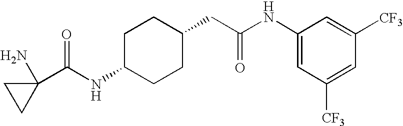 | 452.17 |
| 150 | N-(3,5-bis(trifluoromethyl)phenyl)-2- (cis-4-(2-hydroxy-3,3- dimethylbutylamino)cyclohexyl) acetamide | 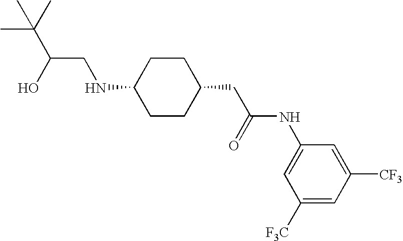 | 469.22 |
| 151 | N-(4-p-tolylthiazol-2-yl)-2-(trans-4- (3,3,3-trifluoro-2-hydroxy-2- (trifluoromethyl)propylamino)cyclohexyl) acetamide | 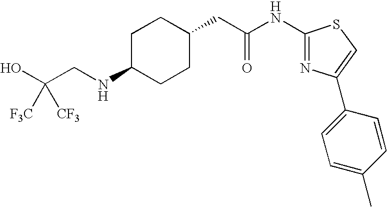 | 510.16 |
| 152 | N-(4-p-tolylthiazol-2-yl)-2-(cis-4-(3,3,3- trifluoro-2-hydroxy-2- (trifluoromethyl)propylamino)cyclohexyl) acetamide | 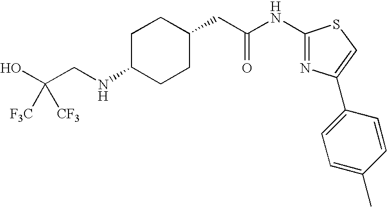 | 510.16 |
| 153 | N-((trans-4-(5-tert-butyl-2- oxooxazolidin-3-yl)cyclohexyl)methyl)- 3,5-bis(trifluoromethyl)benzamide | 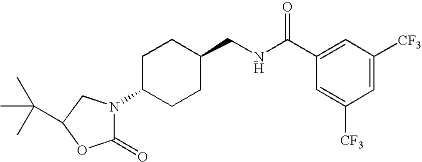 | 495.20 |
| 154 | 3-fluoro-N-((cis-4-(2-oxo-5,5- bis(trifluoromethyl)oxazolidin-3- yl)cyclohexyl)methyl)-5- (trifluoromethyl)benzamide | 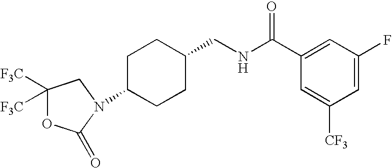 | 525.12 |
| 155 | 3,5-bis(trifluoromethyl)-N-((trans-4-(2- (trifluoromethylsulfonamido)ethylamino) cyclohexyl)methyl)benzamide | 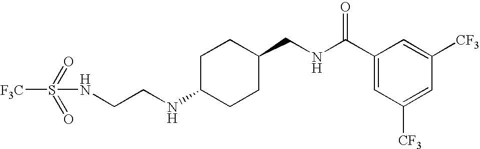 | 544.12 |
| 156 | 3-fluoro-5-(trifluoromethyl)-N-((cis-4-(2- (trifluoromethylsulfonamido)ethylamino) cyclohexyl)methyl)benzamide | 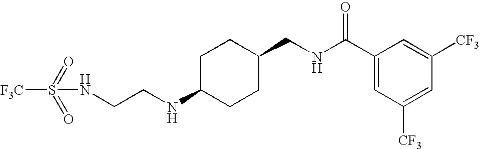 | 494.13 |
| 157 | N-((trans-4-((2-oxo-2-(2,2,2- trifluoroethylamino)ethylamino)methyl) cyclohexyl)methyl)-3,5- bis(trifluoromethyl)benzamide | 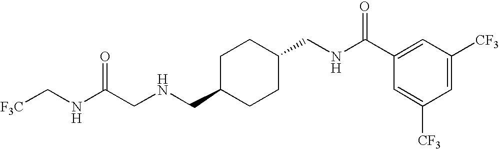 | 522.17 |
| 158 | N-((trans-4-(((5-tert-butyl-1,2,4- oxadiazol-3- yl)methylamino)methyl)cyclohexyl) methyl)-3,5-bis(trifluoromethyl)benzamide | 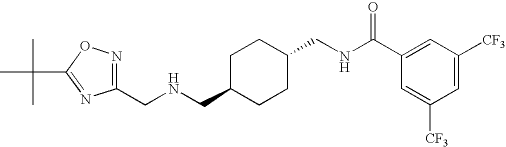 | 521.23 |
| 159 | N-((trans-4-(((5-methylisoxazol-3- yl)methylamino)methyl)cyclohexyl) methyl)-3,5-bis(trifluoromethyl)benzamide | 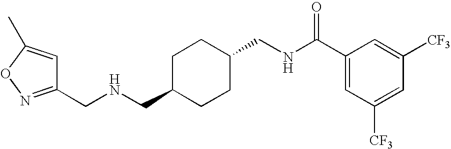 | 478.19 |
| 160 | N-((trans-4-((2-(5-methylpyridin-2- ylamino)-2- oxoethylamino)methyl)cyclohexyl) methyl)-3,5-bis(trifluoromethyl)benzamide | 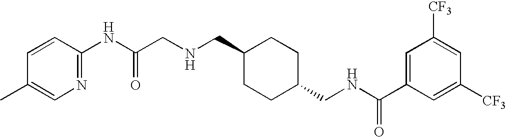 | 531.21 |
| 161 | N-((trans-4-((2-oxo-2-(pentan-3- ylamino)ethylamino)methyl)cyclohexyl) methyl)-3,5- bis(trifluoromethyl)benzamide | 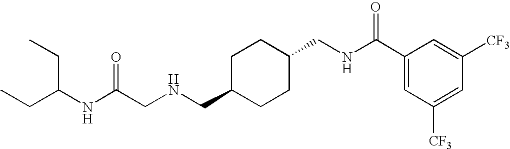 | 510.25 |
| 162 | N-((trans-4-((2-(isobutylamino)-2- oxoethylamino)methyl)cyclohexyl) methyl)-3,5-bis(trifluoromethyl)benzamide | 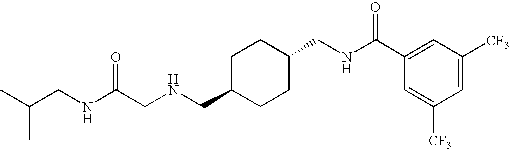 | 496.23 |
| 163 | N-((trans-4-((2-(3-ethylpent-1-yn-3- ylamino)-2- oxoethylamino)methyl)cyclohexyl) methyl)-3,5-bis(trifluoromethyl)benzamide | 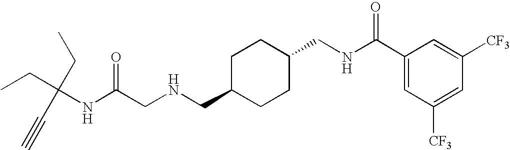 | 534.25 |
| 164 | N-((trans-4-((2-(tert-butylamino)-2- oxoethylamino)methyl)cyclohexyl) methyl)-3,5-bis(trifluoromethyl)benzamide | 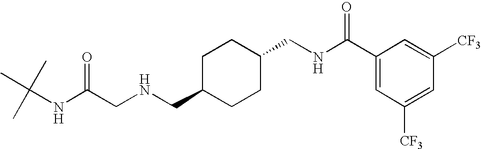 | 496.23 |
| 165 | N-((trans-4-((2- (cyclohexylmethylamino)-2- oxoethylamino)methyl)cyclohexyl) methyl)-3,5-bis(trifluoromethyl)benzamide | 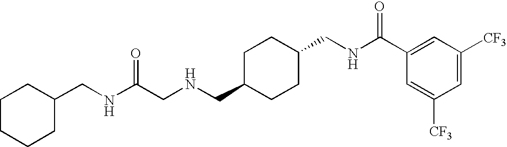 | 536.26 |
| 166 | N-((trans-4-((2-((4- methoxycyclohexyl)(methyl)amino)-2- oxoethylamino)methyl)cyclohexyl) methyl)-3,5-bis(trifluoromethyl)benzamide | 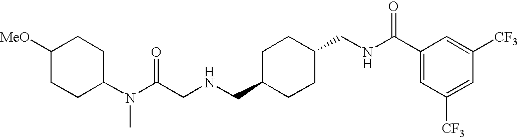 | 566.27 |
| 167 | N-((trans-4-((2- (cyclopentyl(methyl)amino)-2- oxoethylamino)methyl)cyclohexyl) methyl)-3,5-bis(trifluoromethyl)benzamide | 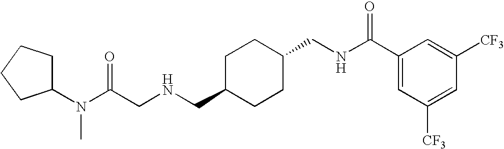 | 522.25 |
| 168 | N-((trans-4-((2- (cyclohexyl(methyl)amino-2- oxoethylamino)methyl)cyclohexyl) methyl)-3,5-bis(trifluoromethyl)benzamide | 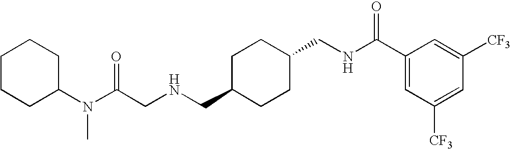 | 536.26 |
| 169 | N-((trans-4-((2-(isopropylamino)-2- oxoethylamino)methyl)cyclohexyl) methyl)-3,5-bis(trifluoromethyl)benzamide | 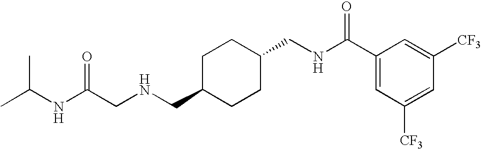 | 482.22 |
| 170 | N-((trans-4-4((2-(sec-butylamino)-2- oxoethylamino)methyl)cyclohexyl) methyl)-3,5-bis(trifluoromethyl)benzamide | 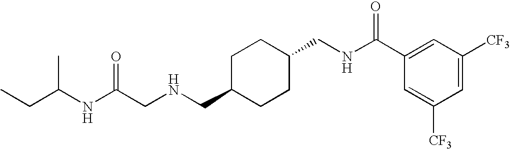 | 496.23 |
| 171 | N-((trans-4-((2-(2-ethylpyrrolidin-1-yl)- 2- oxoethylamino)methyl)cyclohexyl) methyl)-3,5-bis(trifluoromethyl)benzamide | 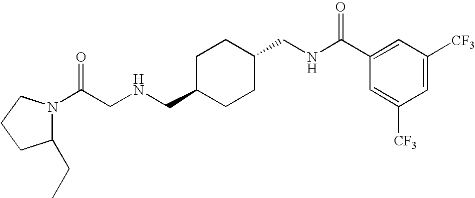 | 522.25 |
| 172 | N-((trans-4-((2-(4-methyl-1,2,5- oxadiazol-3-ylamino)-2- oxoethylamino)methyl)cyclohexyl) methyl)-3,5-bis(trifluoromethyl)benzamide | 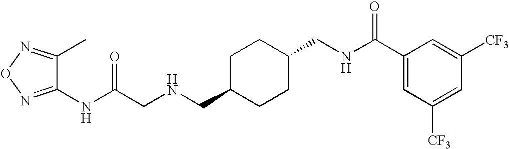 | 522.19 |
| 173 | N-((trans-4-((2-(diethylamino)-2- oxoethylamino)methyl)cyclohexyl) methyl)-3,5-bis(trifluoromethyl)benzamide | 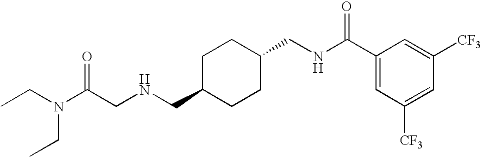 | 496.23 |
| 174 | N-((trans-4-((2-(2-methylpiperidin-1- yl)-2- oxoethylamino)methyl)cyclohexyl) methyl)-3,5-bis(trifluoromethyl)benzamide | 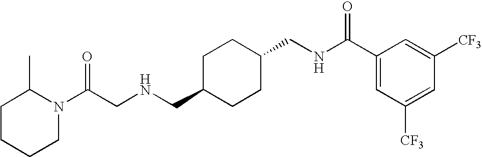 | 522.25 |
| 175 | N-((trans-4-(((2-methylthiazol-4- yl)methylamino)methyl)cyclohexyl) methyl)-3,5-bis(trifluoromethyl) benzamide | 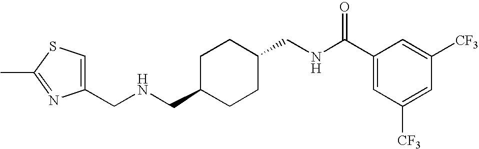 | 494.16 |
| 176 | 2-(4-(2-oxo-5,5- bis(trifluoromethyl)oxazolidin-3- yl)cyclohexyl)-N-(4-p-tolylthiazol-2- yl)acetamide | 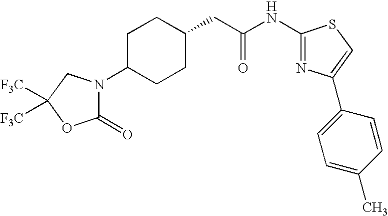 | 536.14 |
| 177 | tert-butyl (trans-4-(2-(3,5- bis(trifluoromethyl)phenylamino)-2- oxoethyl)cyclohexylcarbamate | 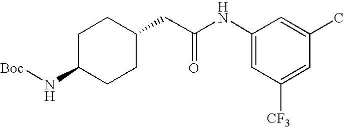 | 469.18 |
| 178 | 2-((trans-4-aminocyclohexyl)-N-(3,5- bis(trifluoromethyl)phenyl)acetamide | 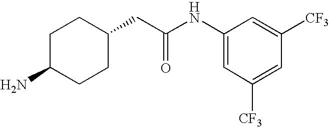 | 369.13 |
| 179 | N-(((1s,4s)-4-(2-(tert-butylamino)-2- oxoethylamino)cyclohexyl)methyl)-3- chlorobenzamide | 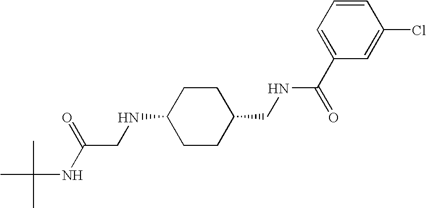 | 380.20 |
| 180 | N-(((1s,4s)-4-(2-(tert-butylamino)-2- oxoethylamino)cyclohexyl)methyl)-3,5- dimethylbenzamide | 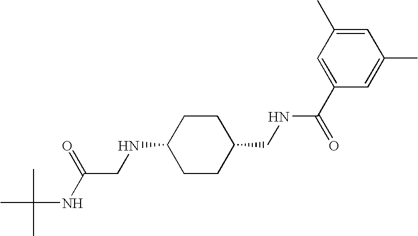 | 374.27 |
| 181 | N-(((1s,4s)-4-(2-(tert-butylamino)-2- oxoethylamino)cyclohexyl)methyl)-3,5- dimethoxybenzamide | 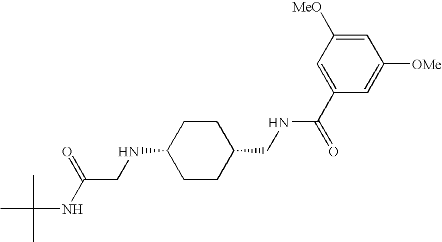 | 406.26 |
| 182 | N-(((1s,4s)-4-(2-(tert-butylamino)-2- oxoethylamino)cyclohexyl)methyl)-3- (trifluoromethyl)benzamide | 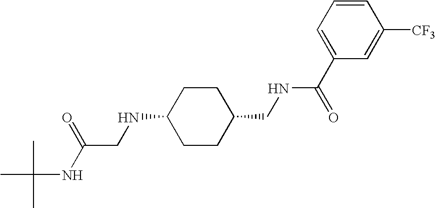 | 414.23 |
| 183 | 3-bromo-N-(((1s,4s)-4-(2-(tert-butylamino)- 2-oxoethylamino)cyclohexyl)methyl) benzamide | 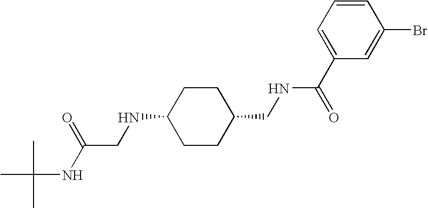 | 424.15 |
| 184 | N-(((1s,4s)-4-(2-(tert-butylamino)-2- oxoethylamino)cyclohexyl)methyl)-3-fluoro- 5-methoxybenzamide | 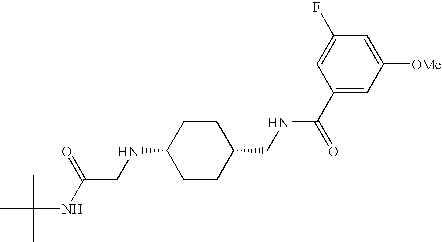 | 394.24 |
| 185 | N-(((1s,4s)-4-(2-(tert-butylamino)-2- oxoethylamino)cyclohexyl)methyl)-3- (trifluoromethoxy)benzamide | 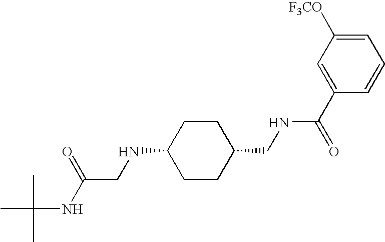 | 430.22 |
| 186 | N-(((1s,4s)-4-(2-(tert-butylamino)-2- oxoethylamino)cyclohexyl)methyl)-3-chloro- 5-fluorobenzamide | 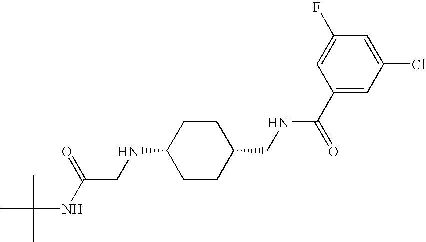 | 398.19 |
| 187 | N-(((1s,4s)-4-(2-(tert-butylamino)-2- oxoethylamino)cyclohexyl)methyl)-3,5- dichlorobenzamide | 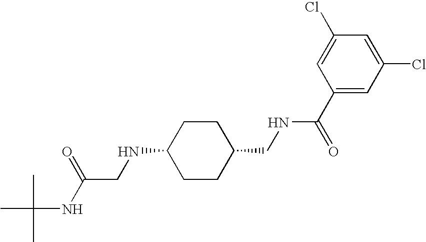 | 414.16 |
| 188 | N-(((1s,4s)-4-(2-(tert-butylamino)-2- oxoethylamino)cyclohexyl)methyl)-3-fluoro- 5-methylbenzamide | 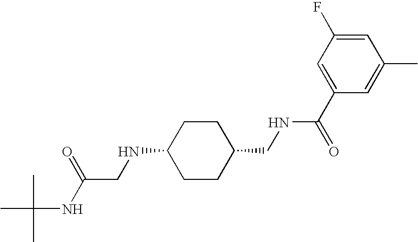 | 378.25 |
| 189 | N-(((1r,4r)-4-(2-(tert-butylamino)-2- oxoethylamino)cyclohexyl)methyl)-3- (trifluoromethoxy)benzamide | 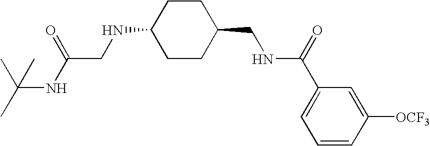 | 430.22 |
| 190 | N-(((1r,4r)-4-(2-(1-methylcyclobutylamino)-2- oxoethylamino)cyclohexyl) methyl)-3- (trifluoromethoxy)benzamide | 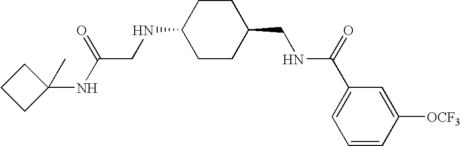 | 442.22 |
| 191 | N-(((1r,4r)-4-(2-(tert-butylamino)-2- oxoethylamino)cyclohexyl)methyl)-3,5- dichlorobenzamide | 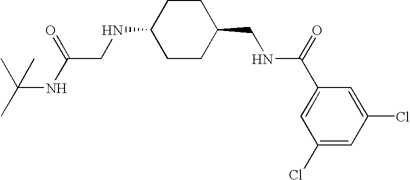 | 414.16 |
| 192 | 3,5-dichloro-N-(((1r,4r)-4-(2-(1- methylcyclobutylamino)-2- oxoethylamino)cyclohexyl)methyl)benzamide | 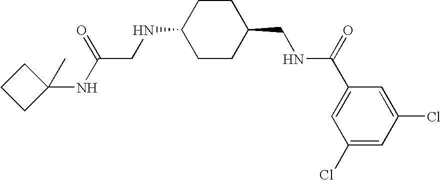 | 426.16 |
| 193 | 3-chloro-5-fluoro-N-(((1s,4s)-4-(2-(1- methylcyclobutylamino)-2- oxoethylamino)cyclohexyl)methyl) benzamide | 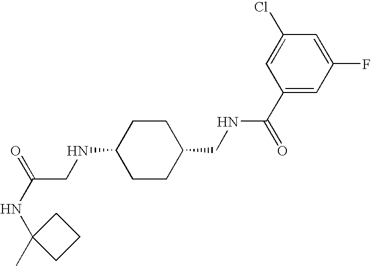 | 410.19 |
| 194 | 3-chloro-N-(((1s,4s)-4-(2-(3,3- dimethylmorpholino)-2- oxoethylamino)cyclohexyl)methyl)-5- fluorobenzamide | 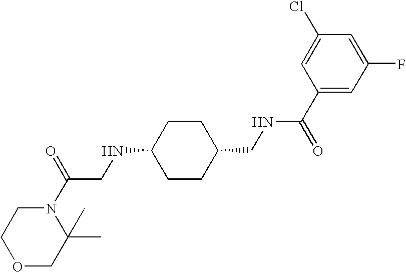 | 440.20 |
| 195 | 3-chloro-N-(((1s,4s)-4-(2-(2,2- dimethylpyrrolidin-1-yl)-2- oxoethylamino)cyclohexyl)methyl)-5- fluorobenzamide | 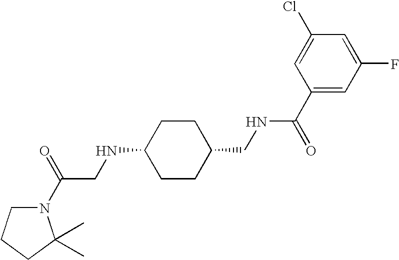 | 424.21 |
| 196 | N-(((1s,4s)-4-(2-(tert-butyl(methyl)amino)-2- oxoethylamino)cyclohexyl)methyl)-3-chloro- 5-fluorobenzamide | 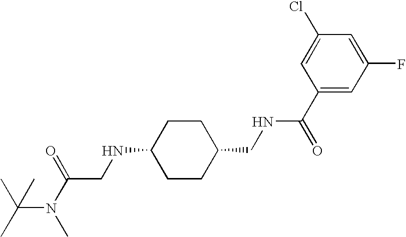 | 412.21 |
| 197 | 3-fluoro-5-methoxy-N-(((1s,4s)-4-(2-(1- methylcyclobutylamino)-2- oxoethylamino)cyclohexyl)methyl) benzamide | 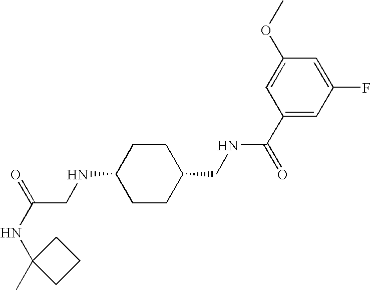 | 406.24 |
| 198 | N-(((1s,4s)-4-(2-(3,3-dimethylmorpholino)-2- oxoethylamino)cyclohexyl)methyl)-3-fluoro- 5-methoxybenzamide | 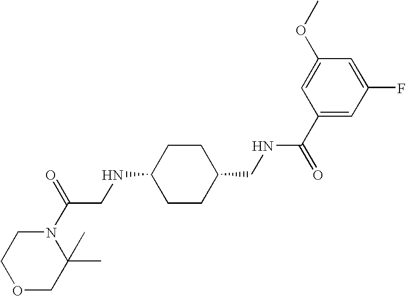 | 436.25 |
| 199 | N-(((1s,4s)-4-(2-(2,2-dimethylpyrrolidin-1-yl)- 2-oxoethylamino)cyclohexyl)methyl)-3- fluoro-5-methoxybenzamide | 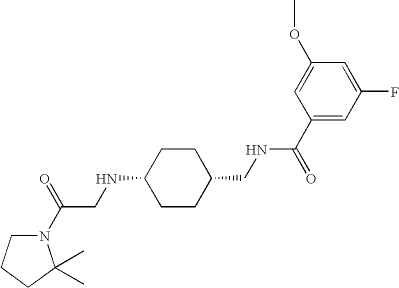 | 420.26 |
| 200 | N-(((1s,4s)-4-(2-(tert-butyl(methyl)amino)-2- oxoethylamino)cyclohexyl)methyl)-3-fluoro- 5-methoxybenzamide | 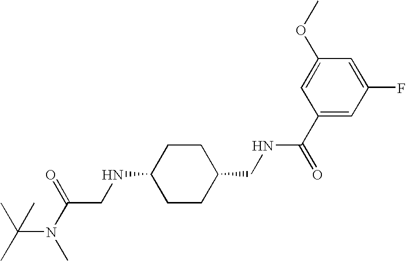 | 408.26 |
| 201 | N-(((1r,4r)-4-(2-(tert-butylamino)-2- oxoethylamino)cyclohexyl)methyl)-3-chloro- 5-fluorobenzamide | 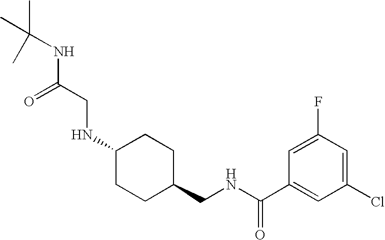 | 398.19 |
| 202 | 3-chloro-5-fluoro-N-(((1r,4r)-4-(2-(1- methylcyclobutylamino)-2- oxoethylamino)cyclohexyl)methyl) benzamide | 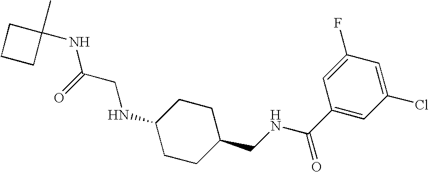 | 410.19 |
| 203 | 3-chloro-N-(((1r,4r)-4-(2-(3,3- dimethylmorpholino)-2- oxoethylamino)cyclohexyl)methyl)-5- fluorobenzamide | 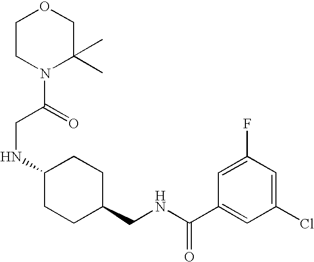 | 440.20 |
| 204 | 3-chloro-N-(((1r,4r)-4-(2-(2,2- dimethylpyrrolidin-1-yl)-2- oxoethylamino)cyclohexyl)methyl)-5- fluorobenzamide | 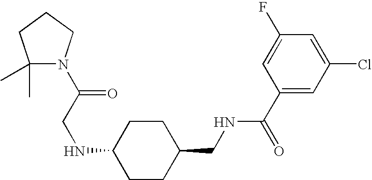 | 424.21 |
| 205 | N-(((1r,4r)-4-(2-(tert-butyl(methyl)amino)-2- oxoethylamino)cyclohexyl)methyl)-3-chloro- 5-fluorobenzamide | 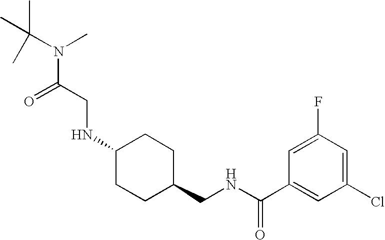 | 412.21 |
| 206 | N-(((1r,4r)-4-(2-(tert-butylamino)-2- oxoethylamino)cyclohexyl)methyl)-3-fluoro- 5-methoxybenzamide | 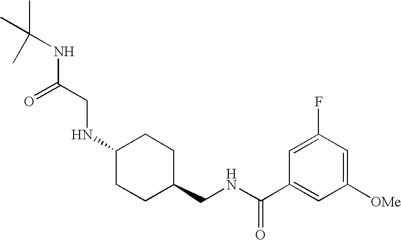 | 394.24 |
| 207 | 3-fluoro-5-methoxy-N-(((1r,4r)-4-(2-(1- methylcyclobutylamino)-2- oxoethylamino)cyclohexyl)methyl) benzamide | 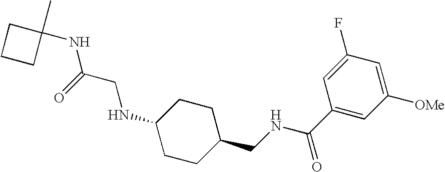 | 406.24 |
| 208 | N-(((1r,4r)-4-(2-(3,3-dimethylmorpholino)-2- oxoethylamino)cyclohexyl)methyl)-3-fluoro- 5-methoxybenzamide | 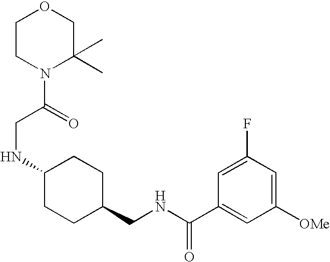 | 436.25 |
| 209 | N-(((1r,4r)-4-(2-(2,2-dimethylpyrrolidin-1-yl)- 2-oxoethylamino)cyclohexyl)methyl)-3- fluoro-5-methoxybenzamide | 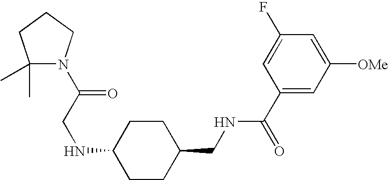 | 420.26 |
| 210 | N-(((1r,4r)-4-(2-(tert-butyl(methyl)amino)-2- oxoethylamino)cyclohexyl)methyl)-3-fluoro- 5-methoxybenzamide | 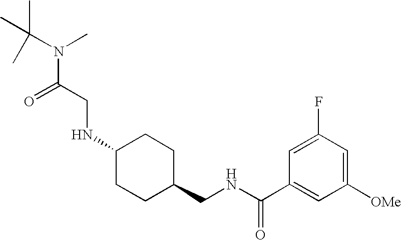 | 408.26 |
| 211 | 3-chloro-5-fluoro-N-(((1r,4r)-4-(2-(methyl(1- methylcyclobutyl)amino)-2- oxoethylamino)cyclohexyl)methyl) benzamide | 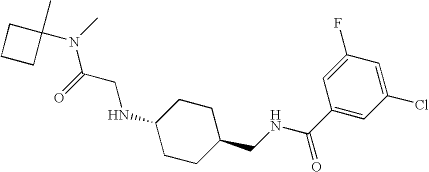 | 424.21 |
| 212 | 3-chloro-5-fluoro-N-(((1s,4s)-4-(2-((1- methoxy-2-methylpropan-2- yl)(methyl)amino)-2- oxoethylamino)cyclohexyl)methyl) benzamide | 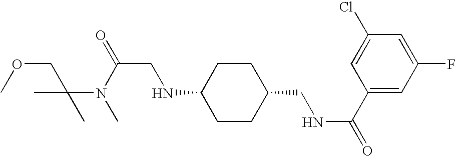 | 442.22 |
| 213 | 3-chloro-5-fluoro-N-(((1s,4s)-4-(2-(methyl(1- methylcyclobutyl)amino)-2- oxoethylamino)cyclohexyl)methyl) benzamide | 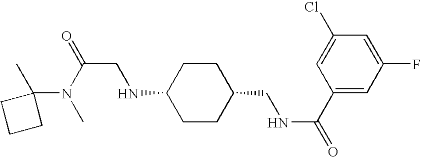 | 424.21 |
| 214 | 3-fluoro-5-methoxy-N-(((1s,4s)-4-(2-((1- methoxy-2-methylpropan-2- yl)(methyl)amino)-2- oxoethylamino)cyclohexyl)methyl) benzamide | 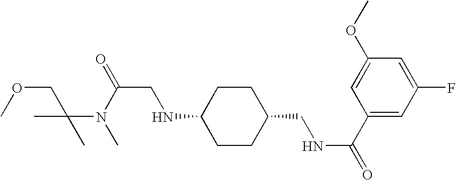 | 438.27 |
| 215 | 3-fluoro-5-methoxy-N-(((1s,4s)-4-(2- (methyl(1-methylcyclobutyl)amino)-2- oxoethylamino)cyclohexyl)methyl) benzamide | 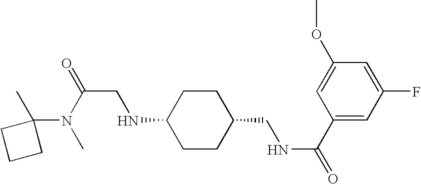 | 420.26 |
| 216 | 3-chloro-5-fluoro-N-(((1s,4s)-4-(2- (methyl(1,1,1-trifluoro-2-methylpropan-2- yl)amino)-2- oxoethylamino)cyclohexyl)methyl) benzamide | 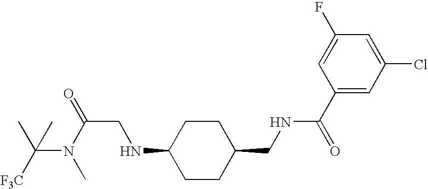 | 466.18 |

### EXAMPLE 15

### T-type Channel Blocking Activities of Various Invention Compounds

A. Transformation of HEK Cells:

T-type calcium channel blocking activity was assayed in human embryonic kidney cells, HEK 293, stably transfected with the T-type calcium channel subunits. Briefly, cells were cultured in Dulbecco's modified eagle medium (DMEM) supplemented with 10% fetal bovine serum, 200 U/ml penicillin and 0.2 mg/ml streptomycin at 37° C. with 5% CO₂. At 85% confluency cells were split with 0.25% trypsin/1 mM EDTA and plated at 10% confluency on glass coverslips. At 12 hours the medium was replaced and the cells stably transfected using a standard calcium phosphate protocol and the appropriate calcium channel cDNA's. Fresh DMEM was supplied and the cells transferred to 28° C./5% CO₂. Cells were incubated for 1 to 2 days prior to whole cell recording.

Standard patch-clamp techniques were employed to identify blockers of T-type currents. Briefly, previously described HEK cell lines stably expressing human α_{1G}, α_{1H} and α_{1I} T-type channels were used for all the recordings (passage #: 4-20, 37° C., 5% CO₂). Whole cell patch clamp experiments were performed using an Axopatch 200B amplifier (Axon Instruments, Burlingame, Calif.) linked to a personal computer equipped with pCLAMP software. Data were analyzed using Clampfit (Axon Instruments) and SigmaPlot 4.0 (Jandel Scientific). To obtain T-type currents, plastic dishes containing semi-confluent cells were positioned on the stage of a ZEISS AXIOVERT S100 microscope after replacing the culture medium with external solution (see below). Whole-cell patches were obtained using pipettes (borosilicate glass with filament, O.D.: 1.5 mm, I.D.: 0.86 mm, 10 cm length), fabricated on a SUTTER P-97 puller with resistance values of ˜5 MΩ (see below for internal solution).

TABLE 2
External Solution 500 ml - pH 7.4, 265.5 mOsm

| Salt | Final mM | Stock M | Final ml | |
|---|---|---|---|---|
| CsCl | 142 | 1 | 71 | |
| CaCl₂ | 2 | 1 | 1 | |
| MgCl₂ | 1 | 1 | 0.5 | |
| HEPES | 10 | 0.5 | 10 | |
| glucose | 10 | — | 0.9 | grams |

TABLE 3
Internal Solution 50 ml - pH 7.3 with CsOH, 270 mOsm

| Salt | Final mM | Stock M | Final ml |
|---|---|---|---|
| Cs-Methanesulfonate | 126.5 | — | 1.442 gr/50 ml |
| MgCl2 | 2 | 1 | 0.1 |
| HEPES | 10 | 0.5 | 1 |
| EGTA-Cs | 11 | 0.25 | 2.2 |
| ATP | 2 | 0.2 | 0.025 |
| | | | (1 aliquot/2.5 ml) |

T-type currents were reliably obtained by using two voltage protocols:
(1) “non-inactivating”, and
(2) “inactivation”

In the non-inactivating protocol, the holding potential is set at −110 mV and with a pre-pulse at −100 mV for 1 second prior to the test pulse at −40 mV for 50 ms. In the inactivation protocol, the pre-pulse is at approximately −85 mV for 1 second, which inactivates about 15% of the T-type channels.

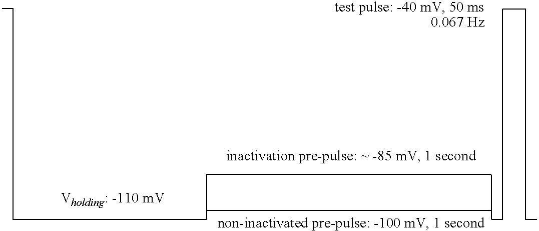

Test compounds were dissolved in external solution, 0.1-0.01% DMSO. After 10 min rest, they were applied by gravity close to the cell using a WPI microfil tubing. The “non-inactivated” pre-pulse was used to examine the resting block of a compound. The “inactivated” protocol was employed to study voltage-dependent block. However, the initial data shown below were mainly obtained using the non-inactivated protocol only. . IC₅₀ values are shown for various compounds of the invention in Table 4 for the drug of interest. Values are shown in μM and values above 10 μM are simply represented as 10 μM. Similarly, IC₅₀ values for α_{1G} below 0.30 μM are simply represented as 0.30 μM.

TABLE 4
T-type Calcium Channel Block

| Compound | α_{1G}(μM) | α_{1H}(μM) |
|---|---|---|
| 1 | 0.39 | 0.58 |
| 2 | 0.45 | 0.55 |
| 3 | 0.47 | 1.02 |
| 4 | 4.54 | 4.43 |
| 5 | | 0.25 |
| 6 | | 0.28 |
| 7 | 0.30 | 1.54 |
| 8 | 3.35 | 4.84 |
| 9 | 0.85 | 0.71 |
| 10 | 0.47 | 0.32 |
| 11 | 1.57 | 2.33 |
| 13 | 0.46 | 2.83 |
| 14 | 0.30 | 0.56 |
| 15 | 0.78 | 1.65 |
| 16 | 4.44 | 10.00 |
| 17 | 7.73 | 4.94 |
| 18 | 0.46 | 1.37 |
| 19 | 0.53 | 1.01 |
| 20 | 3.32 | 5.35 |
| 21 | 0.84 | 4.58 |
| 22 | 0.99 | 3.08 |
| 23 | 0.91 | 2.87 |
| 24 | 0.34 | 0.59 |
| 25 | 0.30 | 2.45 |
| 27 | 1.97 | 9.76 |
| 28 | 0.27 | 2.24 |
| 29 | 0.30 | 0.80 |
| 30 | 1.06 | 5.83 |
| 31 | 0.30 | 2.47 |
| 32 | 0.33 | 3.45 |
| 33 | 0.30 | 0.71 |
| 34 | 0.30 | 1.97 |
| 35 | 0.39 | 0.77 |
| 36 | | 10.00 |
| 37 | | 5.37 |
| 38 | | 7.93 |
| 39 | 1.33 | 0.79 |
| 40 | 0.30 | 0.19 |
| 41 | 0.30 | 0.33 |
| 42 | 0.30 | 0.12 |
| 43 | 0.91 | 0.45 |
| 44 | 0.30 | 0.26 |
| 45 | 0.30 | 0.45 |
| 47 | 0.30 | 0.01 |
| 48 | 0.35 | 0.25 |
| 50 | 4.28 | 2.47 |
| 51 | 1.15 | 0.82 |
| 52 | 1.24 | 1.33 |
| 54 | 0.30 | 0.21 |
| 55 | 0.30 | 0.37 |
| 56 | 0.44 | 0.59 |
| 57 | 0.30 | 0.43 |
| 58 | 1.00 | 0.53 |
| 59 | 0.30 | 0.62 |
| 60 | 1.30 | 1.89 |
| 61 | 1.59 | 1.73 |
| 62 | 0.30 | 0.31 |
| 65 | 8.02 | 7.36 |
| 66 | 1.34 | 3.05 |
| 67 | 5.59 | 2.89 |
| 68 | 0.30 | 0.56 |
| 70 | 0.33 | 0.21 |
| 71 | 0.30 | 0.27 |
| 72 | 0.30 | 0.23 |
| 73 | 0.31 | 0.70 |
| 74 | 2.35 | 0.65 |
| 75 | 0.81 | 0.43 |
| 76 | 0.30 | 0.615 |
| 77 | 0.73 | 1.14 |
| 82 | 2.54 | 2.09 |
| 89 | 1.37 | 1.95 |
| 90 | 3.47 | 3.86 |
| 93 | 1.38 | 1.71 |
| 96 | 0.54 | 1.04 |
| 97 | 0.90 | 0.90 |
| 98 | 0.41 | 0.78 |
| 99 | | 0.44 |
| 100 | 1.03 | 0.85 |
| 101 | 1.51 | 2.05 |
| 102 | 0.97 | 1.81 |
| 103 | 10.95 | 2.081 |
| 104 | | |
| 105 | 0.65 | 6.07 |
| 106 | 6.11 | |
| 108 | 0.45 | 1.53 |
| 109 | 10.00 | |
| 114 | 10.00 | |
| 116 | 2.89 | 10.00 |
| 117 | 1.07 | 10.00 |
| 118 | 4.89 | |
| 119 | 0.88 | 1.53 |
| 120 | 4.44 | |
| 121 | 0.61 | 10.00 |
| 122 | 0.84 | 3.62 |
| 123 | 0.30 | 2.95 |
| 124 | 0.83 | 10.00 |
| 125 | 0.94 | 2.43 |
| 126 | 1.01 | 2.81 |
| 127 | 1.10 | 4.93 |
| 128 | 5.78 | |
| 129 | 5.29 | |
| 130 | 4.11 | |
| 131 | 7.56 | |
| 132 | 1.09 | 10.00 |
| 133 | 0.96 | 4.28 |
| 134 | 1.28 | 6.12 |
| 135 | 1.11 | 5.65 |
| 136 | 3.81 | |
| 137 | 0.70 | 7.85 |
| 138 | 4.88 | |
| 139 | 1.07 | 7.06 |
| 140 | 9.67 | |
| 141 | 10.00 | |
| 142 | 10.00 | |
| 143 | 1.90 | 9.94 |
| 144 | 3.82 | |
| 145 | 7.60 | |
| 146 | 2.79 | 7.87 |
| 147 | 4.59 | |
| 148 | 2.94 | |
| 149 | 4.07 | |
| 150 | 0.30 | 0.524 |
| 151 | 0.48 | 10.00 |
| 152 | 0.30 | 10.00 |
| 153 | 1.18 | 4.01 |
| 154 | | 6.16 |
| 155 | 2.65 | |
| 156 | 10.00 | |
| 179 | 2.92 | 1.32 |
| 181 | 3.65 | 0.33 |
| 182 | 2.37 | 3.42 |
| 183 | 2.24 | 0.82 |
| 185 | 0.79 | 0.33 |
| 186 | 1.18 | 0.26 |
| 187 | 0.30 | 0.06 |
| 188 | 4.28 | 1.23 |
| 189 | 3.66 | 1.66 |
| 190 | 1.63 | 0.62 |
| 191 | 0.53 | 0.18 |
| 192 | 0.34 | 0.12 |
| 193 | 0.72 | 0.23 |
| 194 | 3.40 | 0.40 |
| 195 | 1.10 | 0.17 |
| 196 | 1.62 | 0.21 |
| 197 | 1.94 | 0.26 |
| 198 | 9.92 | 0.63 |
| 199 | 3.80 | 0.35 |
| 200 | 3.35 | 0.47 |
| 201 | 1.80 | 0.57 |
| 202 | 0.97 | 0.31 |
| 203 | 6.13 | 2.16 |
| 204 | 1.58 | 0.65 |
| 205 | 2.02 | 1.10 |
| 206 | 4.42 | 1.03 |
| 207 | 3.82 | 0.92 |
| 208 | 10.00 | 4.33 |
| 209 | 4.05 | 1.60 |
| 210 | 5.61 | 2.39 |
| 211 | 1.18 | 0.54 |
| 212 | 1.58 | 0.42 |
| 213 | 1.03 | 0.33 |
| 214 | 5.82 | 0.71 |
| 215 | 1.99 | 0.41 |

### EXAMPLE 16

### L5/L6 Spinal Nerve Ligation (SNL)—Chung Pain Model

The Spinal Nerve Ligation is an animal model representing peripheral nerve injury generating a neuropathic pain syndrome. In this model experimental animals develop the clinical symptoms of tactile allodynia and hyperalgesia. L5/L6 Spinal nerve ligation (SNL) injury was induced using the procedure of Kim and Chung (Kim, S. H., et al., Pain (1992) 50:355-363) in male Sprague-Dawley rats (Harlan; Indianapolis, Ind.) weighing 200 to 250 grams.

Anaesthesia was induced with 2% isofluorane in O₂ at 2 L/min and maintained with 0.5% isofluorane in O₂. Rats were then shaved and aseptically prepared for surgeries. A 2 cm paraspinal incision was made at the level of L4-S2. L4/L5 was exposed by removing the transverse process above the nerves with a small rongeur. The L5 spinal nerve is the larger of the two visible nerves below the transverse process and lies closest to the spine. The L6 spinal nerve is located beneath the corner of the slope bone. A home-made glass Chung rod was used to hook L5 or L6 and a pre-made slip knot of 4.0 silk suture was placed on the tip of the rod just above the nerve and pulled underneath to allow for the tight ligation. The L5 and L6 spinal nerves were tightly ligated distal to the dorsal root ganglion. The incision was closed, and the animals were allowed to recover for 5 days. Rats that exhibited motor deficiency (such as paw-dragging) or failure to exhibit subsequent tactile allodynia were excluded from further testing.

Sham control rats underwent the same operation and handling as the experimental animals, but without SNL.

Prior to initiating drug delivery, baseline behavioural testing data is obtained. At selected times after infusion of the Test or Control Article behavioural data can then be collected again.

### A. Assessment of Tactile Allodynia—Von Frey

The assessment of tactile allodynia consisted of measuring the withdrawal threshold of the paw ipsilateral to the site of nerve injury in response to probing with a series of calibrated von Frey filaments (innocuous stimuli). Animals were acclimated to the suspended wire-mesh cages for 30 min before testing. Each von Frey filament was applied perpendicularly to the plantar surface of the ligated paw of rats for 5 sec. A positive response was indicated by a sharp withdrawal of the paw. For rats, the first testing filament is 4.31. Measurements were taken before and after administration of test articles. The paw withdrawal threshold was determined by the non-parametric method of Dixon (Dixon, W., Ann. Rev. Pharmacol. Toxicol. (1980) 20:441-462.), in which the stimulus was incrementally increased until a positive response was obtained, and then decreased until a negative result was observed. The protocol was repeated until three changes in behaviour were determined (“up and down” method) (Chaplan, S.R., et al., J. Neuroscience Methods (1994) 53:55-63.). The 50% paw withdrawal threshold was determined as (10^{[Xf+kδ]})/10,000, where X_{f}=the value of the last von Frey filament employed, k=Dixon value for the positive/negative pattern, and δ=the logarithmic difference between stimuli. The cut-off values for rats were no less than 0.2 g and no higher than 15 g (5.18 filament); for mice no less than 0.03 g and no higher than 2.34 g (4.56 filament). A significant drop of the paw withdrawal threshold compared to the pre-treatment baseline is considered tactile allodynia.

FIG. 1 is a plot of the antiallodynic effects of compounds 1, 9 and vehicle (i.p.) tested in SNL L5/L6 rats using von Frey at 60 minutes compared to baseline and post-SNL.

### B. Assessment of Thermal Hypersensitivity—Hargreaves

The method of Hargreaves and colleagues (Hargreaves, K., et al., Pain (1988) 32:77-8) can be employed to assess paw-withdrawal latency to a noxious thermal stimulus.

Rats were allowed to acclimate within a Plexiglas enclosure on a clear glass plate for 30 minutes. A radiant heat source (i.e., halogen bulb coupled to an infrared filter) was then activated with a timer and focused onto the plantar surface of the affected paw of treated rats. Paw-withdrawal latency can be determined by a photocell that halted both lamp and timer when the paw is withdrawn. The latency to withdrawal of the paw from the radiant heat source was determined prior to L5/L6 SNL, 7-14 days after L5/L6 SNL but before drug, as well as after drug administration. A maximal cut-off of 33 seconds is employed to prevent tissue damage. Paw withdrawal latency can be thus determined to the nearest 0.1 second. A significant drop of the paw withdrawal latency from the baseline indicates the status of thermal hyperalgesia. Antinociception is indicated by a reversal of thermal hyperalgesia to the pre-treatment baseline or a significant (p <0.05) increase in paw withdrawal latency above this baseline. Data is converted to % anti hyperalgesia or % anti nociception by the formula: (100×(test latency−baseline latency)/(cut-off−baseline latency) where cut-off is 21 seconds for determining anti hyperalgesia and 40 seconds for determining anti nociception.

FIG. 2 is a plot of the anti-hyperalgesic effects of compounds 1, 9 and vehicle (i.p.) tested in SNL L5/L6 rats using radiant heat at 60 minutes compared to baseline and post-SNL.

### EXAMPLE 17

### Electroconvulsive Shock (ECS) Threshold Test Epilepsy Model

The proconvulsant or anticonvulsant activity of compounds can be evaluated using the electroconvulsive shock threshold test following the method described by Swinyard et al., (J. Pharmacol. Exp. Ther., 106, 319-330, 1952).

To elicit tonic convulsions a rectangular electroconvulsive shock is administered to OF1 mice for 0.4 s at 50 Hz, via corneal electrodes connected to a constant current shock generator (Ugo Basile: Type 7801). The threshold for tonic convulsions is determined as follows: The first animal is exposed to 30 mA. If the first animal does not exhibit tonic convulsions within 5 seconds, the second animal is exposed to 40 mA, and so on (increments of 10 mA) until the first tonic convulsion is observed. Once the first tonic convulsion is observed, the intensity of ECS is decreased by 5 mA for the next animal and then the intensity is decreased or increased by 5 mA from animal to animal depending on whether the previous animal convulsed or not. The minimum intensity given is 5 mA and the maximum intensity given is 95 mA.

Each treatment group consists of a number mice that are all exposed to ECS, but only the first 3 animals are used to estimate the threshold current and are not included in the analysis.

For optimal results, each test substance is evaluated at multiple doses, administered i.p. or p.o., prior to ECS to coincide with times of peak optimal effect (Tmax), and compared with a vehicle control group. Diazepam administered under the same experimental conditions can be used as reference substance and the vehicle alone can be administered as a vehicle control.

The results are reported as the mean intensity administered, number of deaths and percent change from control for each treatment group for approximately 30 minutes after the animal receives the ECS. A positive percent change indicates an anticonvulsant effect whereas a negative percent change indicates a proconvulsant effect. For the test substances, data (intensity) can be analyzed using a one-way ANOVA followed by Dunnett's t test in case of a significant group effect. The effects of the reference substance (diazepam) can be analyzed using a Student's t test.

1. A method to treat a condition modulated by calcium ion channel activity, which method comprises administering to a subject in need of such treatment an amount of the compound of formula (1) effective to ameliorate said condition, wherein said compound is of the formula: 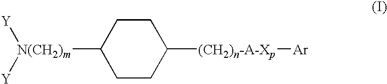
or a pharmaceutically acceptable salt or conjugate thereof, wherein
A is C(O)NH or NHC(O);
X is an optionally substituted alkylene (1-4C), heteroalkylene (2-4C), alkenylene (2-4C), or heteroakenylene (2-4C);
m, n and p are independently 0 or 1;
Ar is an optionally substituted aryl (6-10C) or heteroaryl (5-12 ring members);
each Y is independently H, SR′, SOR′, SO₂R′, wherein each R′ is independently H or an optionally substituted group selected from alkyl (1-6C), alkenyl (2-6C), alkynyl (2-6C), heteroalkyl (2-6C), heteroalkenyl (2-6), heteroalkynyl (2-6C); or each Y is an optionally substituted group selected from alkyl (1-10C), alkenyl (2-10C), alkynyl (2-10C), heteroalkyl (2-10C), heteroalkenyl (2-10C), heteroalkynyl (2-10C), aryl (6-12C)-alkyl (1-6C) or heteroaryl (5-12 ring members)-alkyl (1-6C); or two Y may together form an optionally substituted heterocyclic ring (4-6 ring members);
wherein the optional substituents on X, Y and Ar may be one or more halo, CN, NO₂, CF₃, OCF₃, COOR′, CONR′₂, OR′, SR′, SOR′, SO₂R′, NR′₂, NR′(CO)R′, NR′C(O)OR′, NR′C(O)NR′₂, NR′SO₂NR′₂, NR′SO₂R′, wherein each R′ is independently H or an optionally substituted group selected from alkyl (1-6C), alkenyl (2-6C), alkynyl (2-6C), heteroalkyl (2-6C) heteroalkenyl (2-6), heteroalkynyl (2-6C); or each substituent is alkyl (1-6C), alkenyl (2-6C), alkynyl (2-6C), heteroalkyl (2-6C), heteroalkenyl (2-6C), heteroalkynyl (2-6C); aryl (6-10C), heteroaryl (5-12 ring members), O-aryl (6-10C), O-heteroaryl (5-12 ring members), aryl (6-12C)-alkyl (1-6C) or heteroaryl (5-12 ring members)-alkyl (1-6C); and wherein optional substituents on X and Y may be additionally selected from ═O, ═NOR′.

2. The method of claim 1 wherein said condition is modulated by T-type calcium channel activity.

3. The method of claim 1 wherein said condition is cardiovascular disease, epilepsy, diabetes, cancer, chronic or acute pain, sleep disorders, Parkinson's disease, psychosis, overactive bladder, renal disease, addiction, neuroprotection or male birth control.

4. The method of claim 1 wherein said condition is cardiovascular disease, epilepsy, cancer, or chronic or acute pain.

5. The method of claim 1 wherein Ar is an optionally substituted phenyl, oxadiazolyl, thiazolyl, pyridinyl, or isoxazolyl.

6. The method of claim 1 wherein Ar is an optionally substituted phenyl.

7. The method of claim 1 wherein at least one of m and n is 1.

8. The method of claim 1 wherein A is NHC(O) if n is 0.

9. The method of claim 1 wherein p is 0.

10. The method of claim 1 wherein p is 1.

11. The method of claim 10 wherein X is an optionally substituted alkylene (1-2C) or an optionally substituted alkenylene (2C).

12. The method of claim 10 wherein X is methylene.

13. The method of claim 1 wherein the optional substituents on Ar are independently selected from fluoro, chloro, trifluoromethyl, methyl, ethyl, trifluoromethoxy, t-butyl, t-butyloxy, methoxy, phenyl, or tolyl.

14. The method of claim 1 wherein at least one Y is H.

15. The method of claim 1 wherein one Y is an optionally substituted alkyl (1-10C), heteroalkyl (2-10C), aryl(6-10C)alkyl(1-6C), heteroaryl (5-12 ring members)-alkyl (1-6C).

16. The method of claim 1 wherein Y is an optionally substituted alkyl (1-10C), or heteroalkyl (2-10C).

17. The method of claim 1 wherein Y is an optionally substituted aryl(6-10C)alkyl(1-3C) or heteroaryl(5-12 ring members)-alkyl (1-3C).

18. The method of claim 1 wherein two Y together form an optionally substituted heterocyclic ring (4-6 ring members).

19. The method of claim 1 wherein the compound is of formula 2: 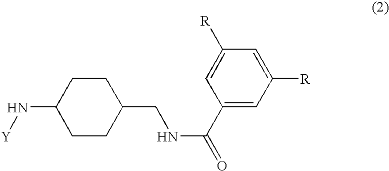
or a pharmaceutically acceptable salt or conjugate thereof, wherein Y is as defined in claim 1 and each R is independently H, fluoro, chloro, trifluoromethyl, methyl, ethyl, trifluoromethoxy, t-butyl, t-butyloxy or methoxy.

20. The method of claim 1 wherein the compound is: 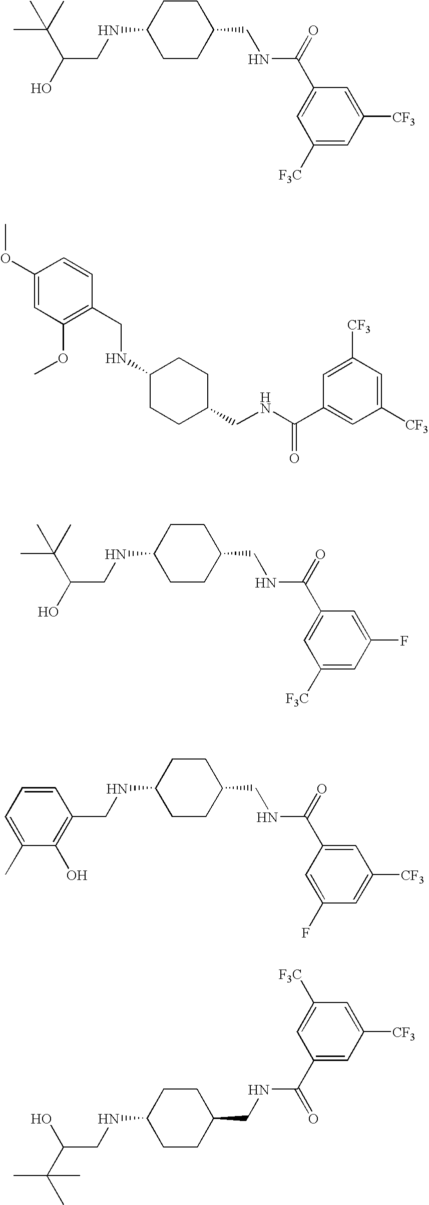 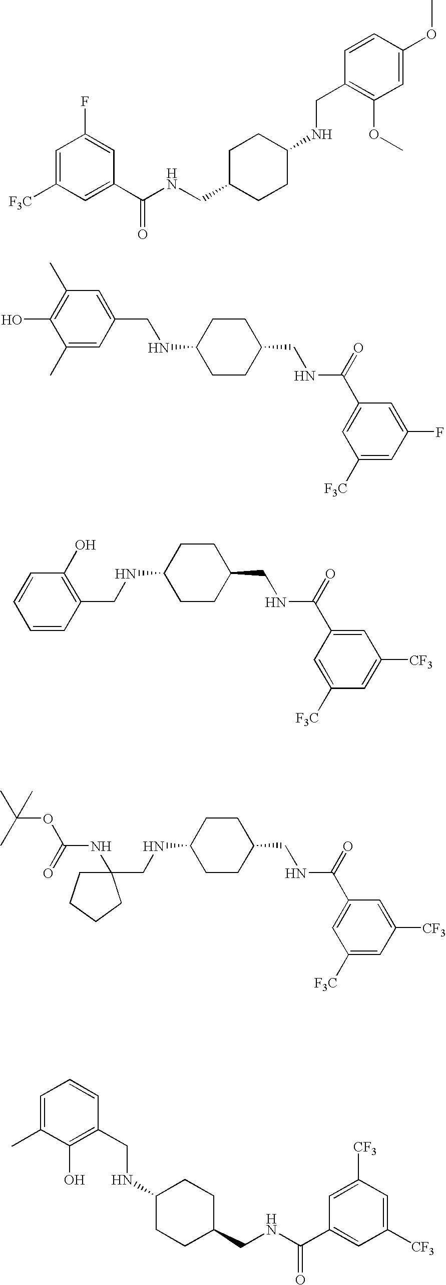 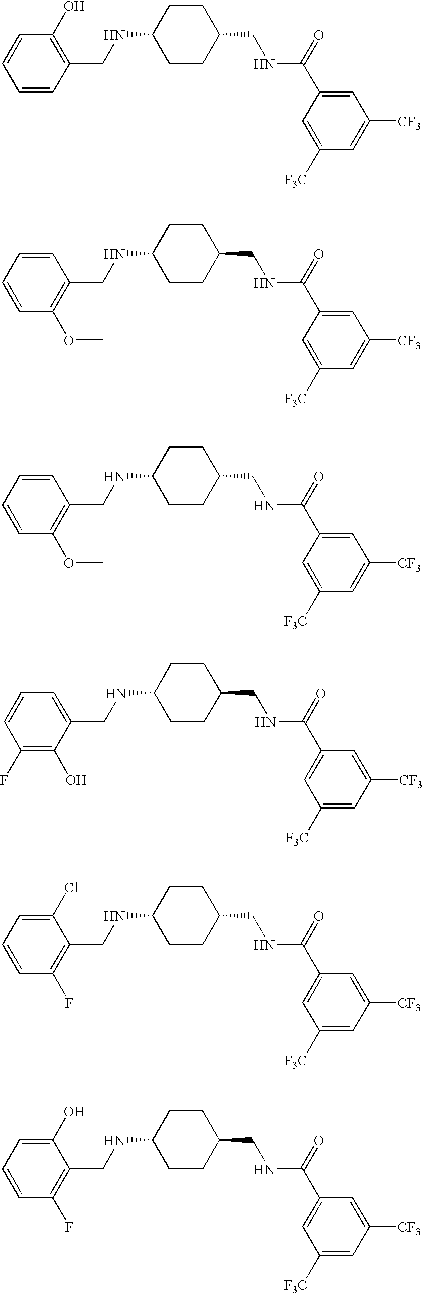 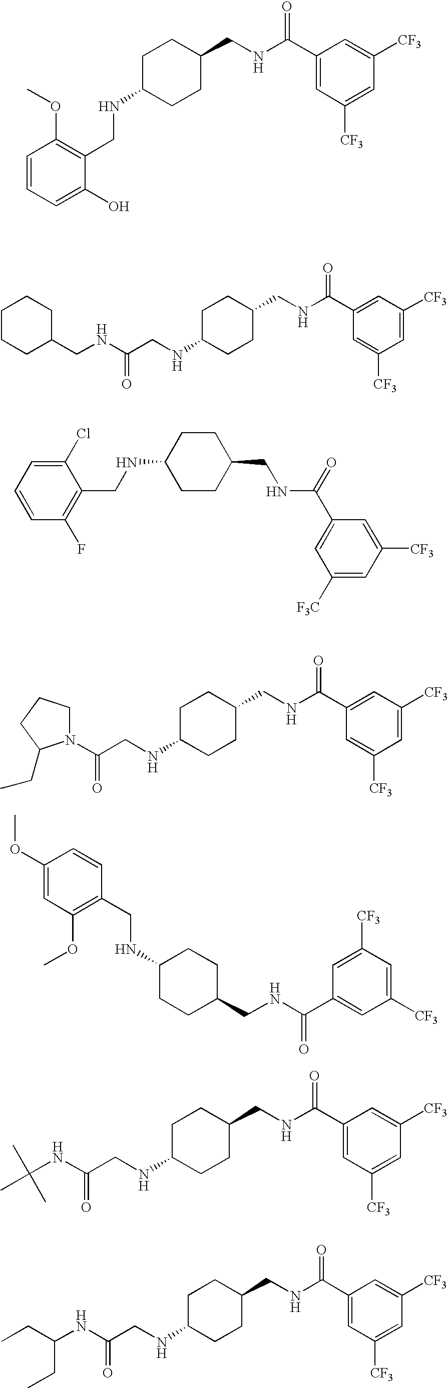 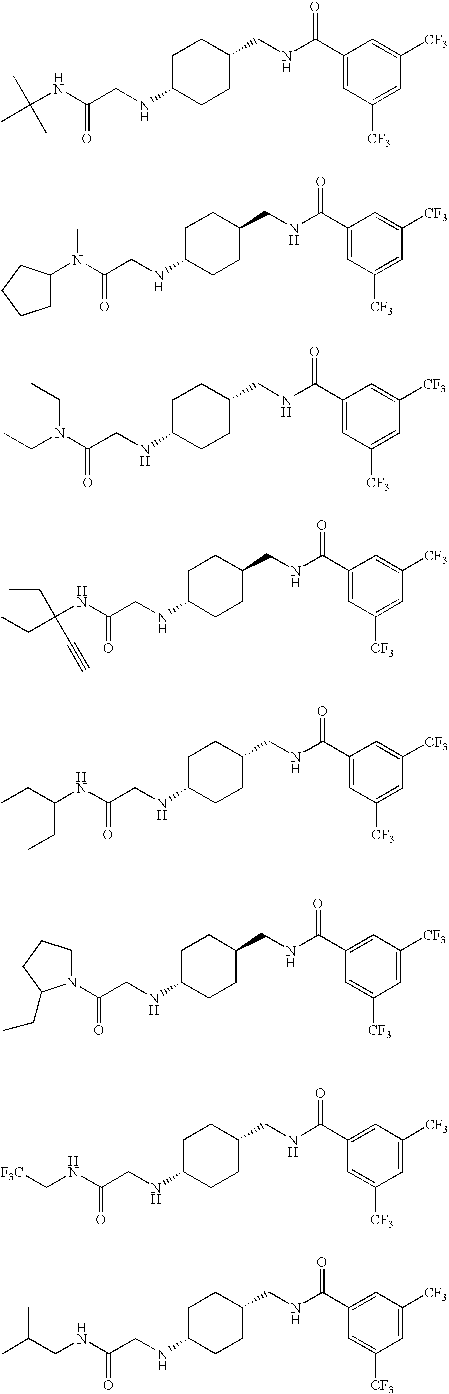 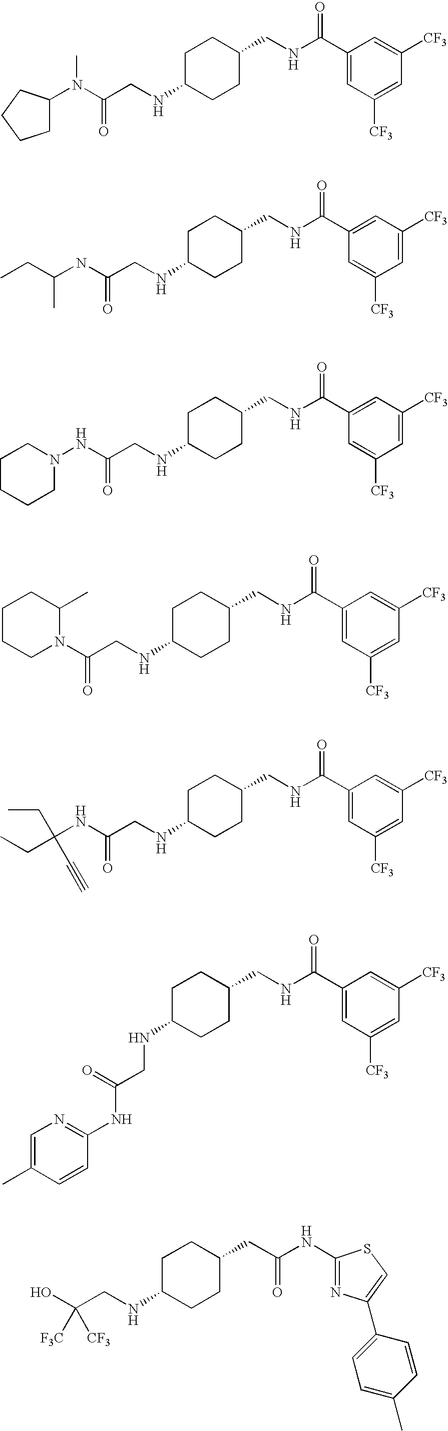 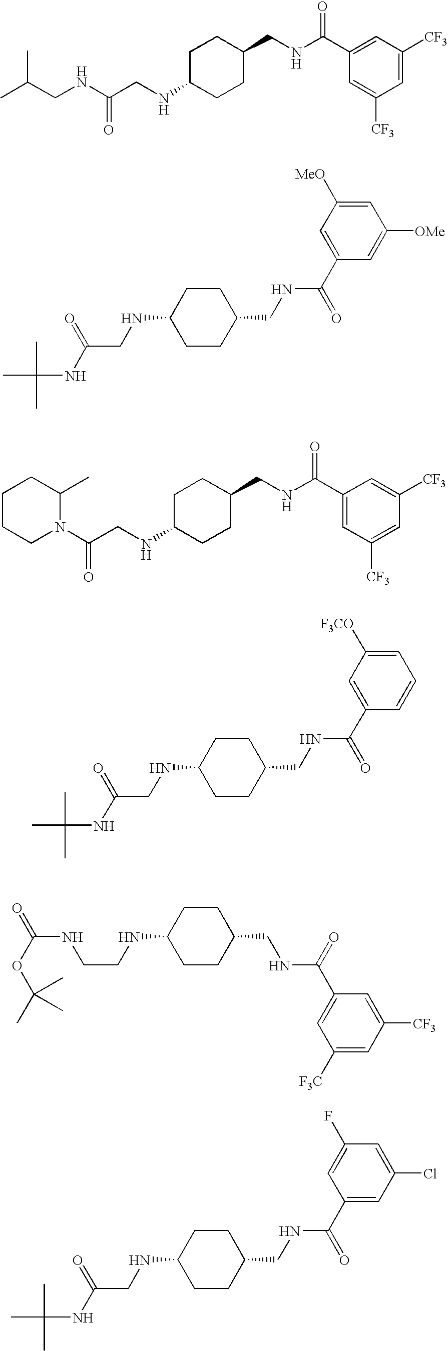 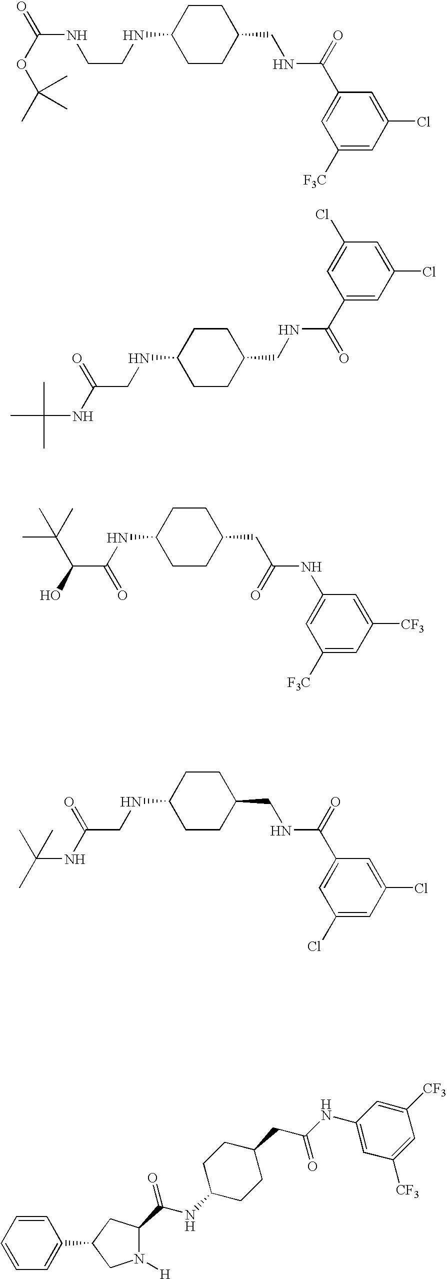 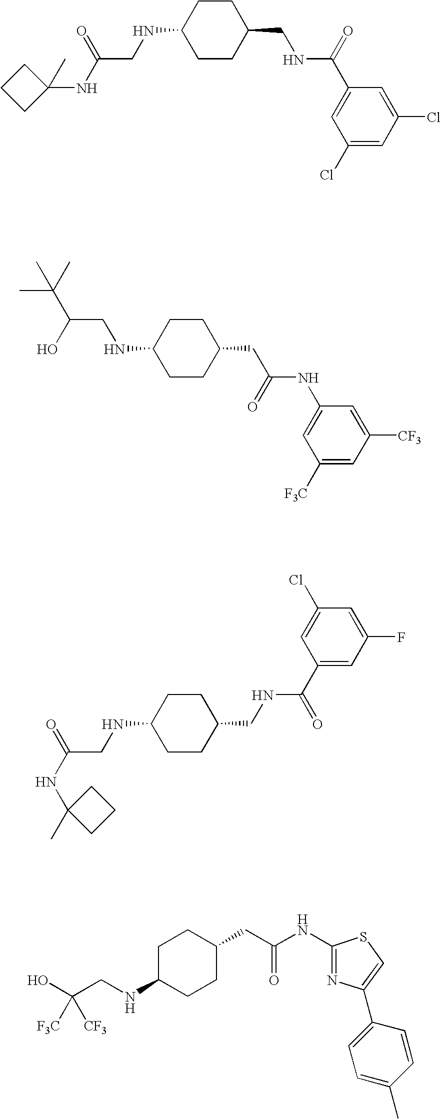 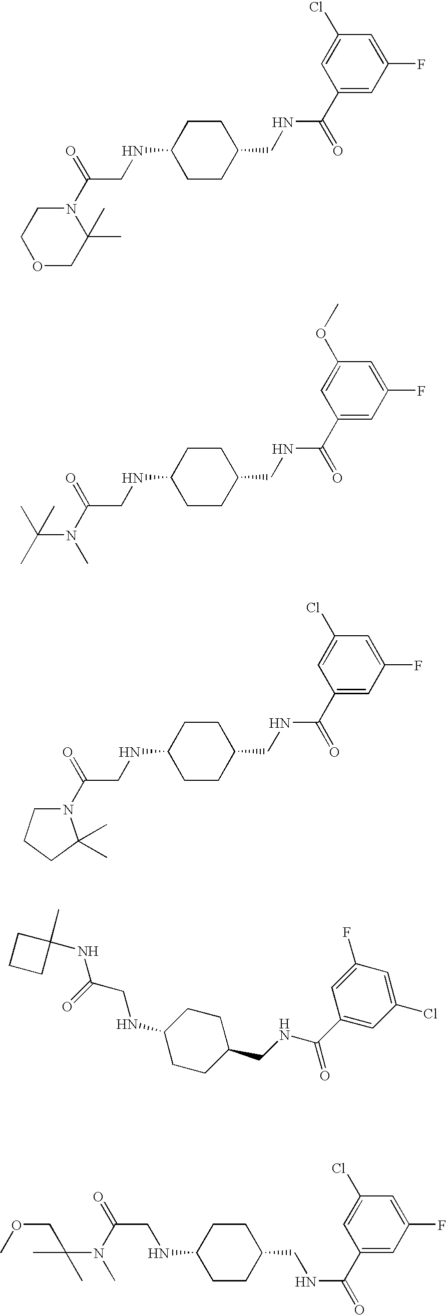 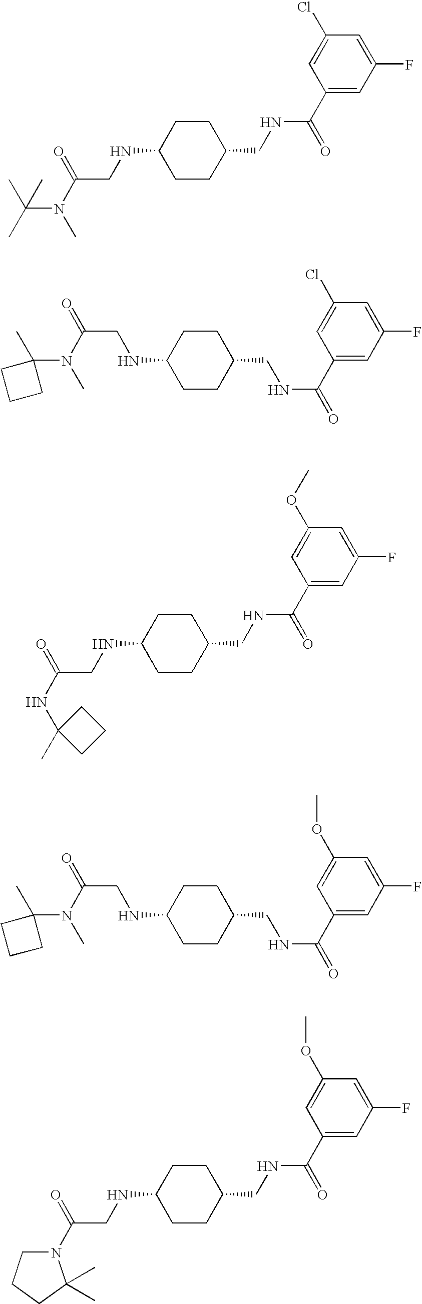
or a pharmaceutically acceptable salt of one of these.

21. A pharmaceutical composition comprising a compound of formula (1): 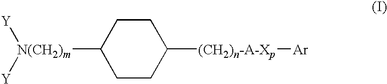
or a pharmaceutically acceptable salt or conjugate thereof, wherein
A is C(O)NH or NHC(O);
X is an optionally substituted alkylene (1-4C), heteroalkylene (2-4C), alkenylene (2-4C), or heteroakenylene (2-4C);
m, n and p are independently 0 or 1;
Ar is an optionally substituted aryl (6-10C) or heteroaryl (5-12 ring members);
each Y is independently H, SR′, SOR′, SO₂R′, wherein each R′ is independently H or an optionally substituted group selected from alkyl (1-6C), alkenyl (2-6C), alkynyl (2-6C), heteroalkyl (2-6C), heteroalkenyl (2-6), heteroalkynyl (2-6C); or each Y is an optionally substituted group selected from alkyl (1-10C), alkenyl (2-10C), alkynyl (2-10C), heteroalkyl (2-10C), heteroalkenyl (2-10C), heteroalkynyl (2-10C), aryl (6-12C)-alkyl (1-6C) or heteroaryl (5-12 ring members)-alkyl (1-6C); or two Y may together form an optionally substituted heterocyclic ring (4-6 ring members);
wherein the optional substituents on X, Y and Ar may be one or more halo, CN, NO₂, CF₃, OCF₃, COOR′, CONR′₂, OR′, SR′, SOR′, SO₂R′, NR′₂, NR′(CO)R′, NR′C(O)OR′, NR′C(O)NR′₂, NR′SO₂NR′₂, NR′SO₂R′, wherein each R′ is independently H or an optionally substituted group selected from alkyl (1-6C), alkenyl (2-6C), alkynyl (2-6C), heteroalkyl (2-6C) heteroalkenyl (2-6), heteroalkynyl (2-6C); or each substituent is alkyl (1-6C), alkenyl (2-6C), alkynyl (2-6C), heteroalkyl (2-6C), heteroalkenyl (2-6C), heteroalkynyl (2-6C); aryl (6-10C), heteroaryl (5-12 ring members), O-aryl (6-10C), O-heteroaryl (5-12 ring members), aryl (6-12C)-alkyl (1-6C) or heteroaryl (5-12 ring members)-alkyl (1-6C); and wherein optional substituents on X and Y may be additionally selected from ═O, ═NOR′;
with the provisos that Ar is not naphthyl and the two Y groups do not together form a pyrrolidin-2-onyl ring.

22. The pharmaceutical composition of claim 21 wherein Ar is an optionally substituted phenyl, oxadiazolyl, thiazolyl, pyridinyl, or isoxazolyl.

23. The pharmaceutical composition of claim 21 wherein Ar is an optionally substituted phenyl.

24. The pharmaceutical composition of claim 21 wherein at least one of m and n is 1.

25. The pharmaceutical composition of claim 21 wherein A is NHC(O) if n is 0.

26. The pharmaceutical composition of claim 21 wherein p is 0.

27. The pharmaceutical composition of claim 21 wherein p is 1.

28. The pharmaceutical composition of claim 27 wherein X is an optionally substituted alkylene (1-2C) or an optionally substituted alkenylene (2C).

29. The pharmaceutical composition of claim 27 wherein X is methylene.

30. The pharmaceutical composition of claim 27 wherein the optional substituents on Ar are independently selected from fluoro, chloro, trifluoromethyl, methyl, ethyl, trifluoromethoxy, t-butyl, t-butyloxy, methoxy, phenyl, or tolyl.

31. The pharmaceutical composition of claim 21 wherein at least one Y is H.

32. The pharmaceutical composition of claim 21 wherein one Y is an optionally substituted alkyl (1-10C), heteroalkyl (2-10C), aryl(6-10C)alkyl(1-6C), or heteroaryl (5-12 ring members)-alkyl (1-6C).

33. The pharmaceutical composition of claim 21 wherein Y is an optionally substituted alkyl (1-10C), or heteroalkyl (2-10C).

34. The pharmaceutical composition of claim 21 wherein Y is an optionally substituted aryl(6-10C)alkyl(1-3C) or heteroaryl(5-12 ring members)-alkyl (1-3C).

35. The pharmaceutical composition of claim 21 wherein two Y together form an optionally substituted heterocyclic ring (4-6 ring members).

36. The pharmaceutical composition of claim 21 wherein the compound is of formula 2: 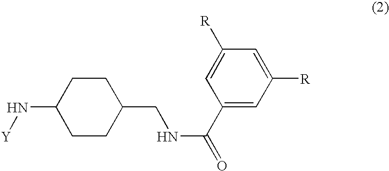
or a pharmaceutically acceptable salt or conjugate thereof, wherein Y is as defined in claim 21 and each R is independently H, fluoro, chloro, trifluoromethyl, methyl, ethyl, trifluoromethoxy, t-butyl, t-butyloxy or methoxy.

37. The pharmaceutical composition of claim 21 wherein the compound is: 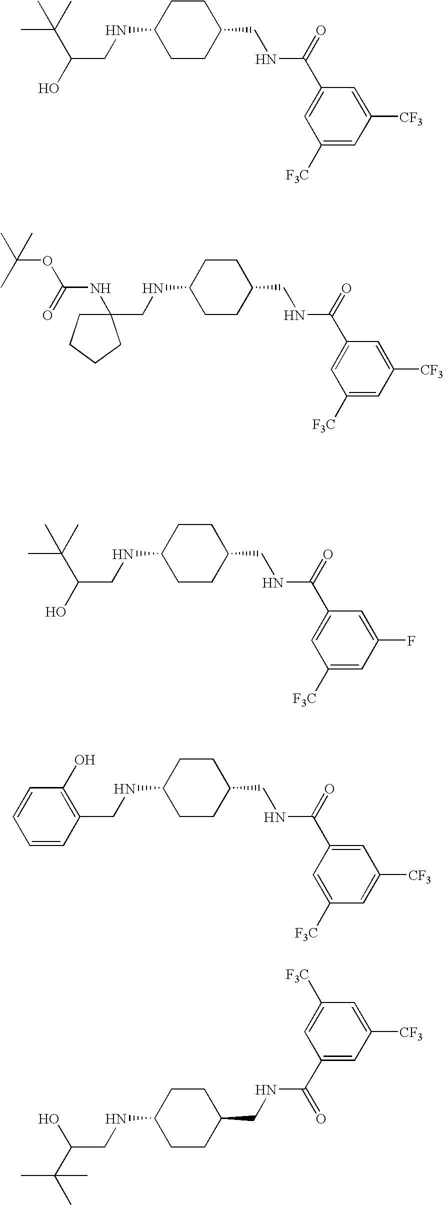 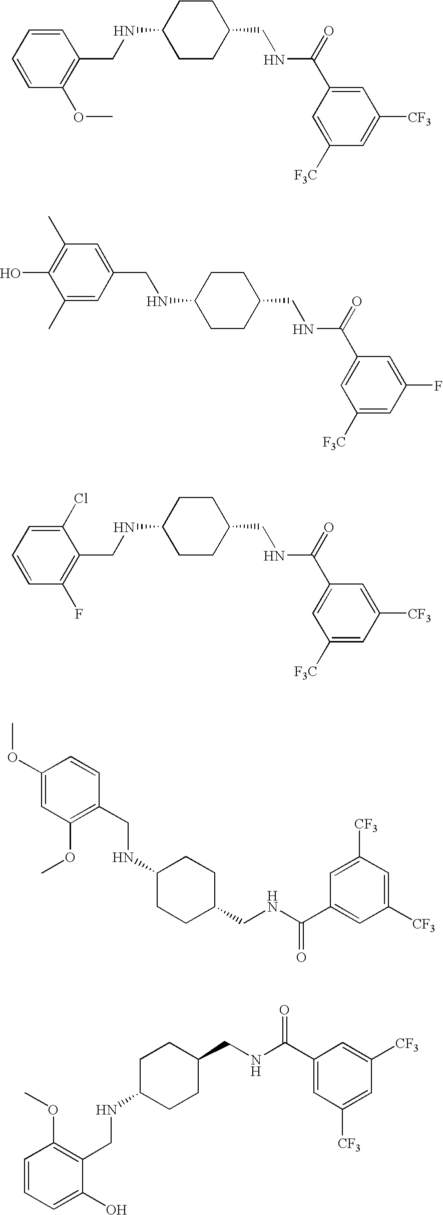 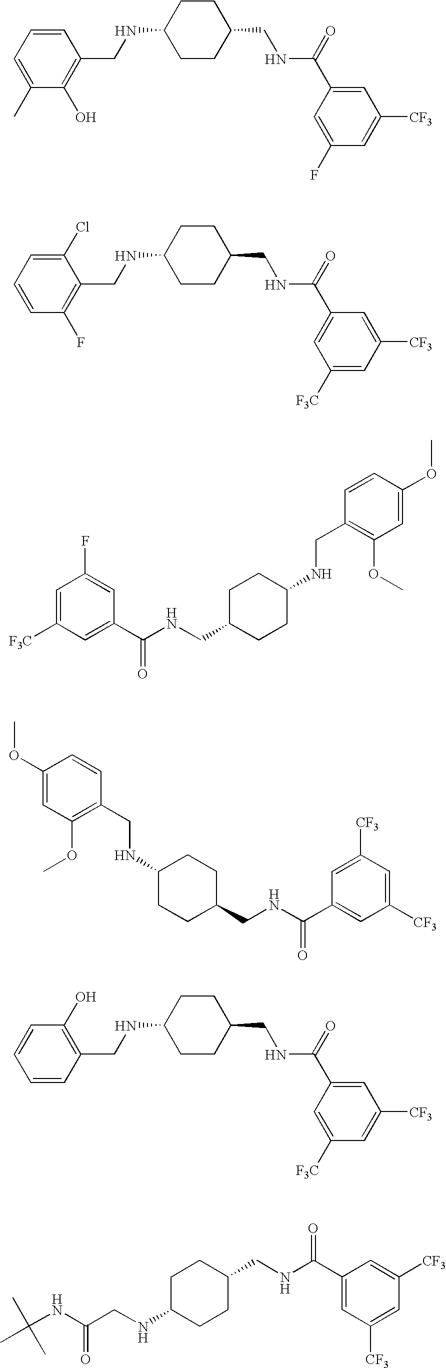 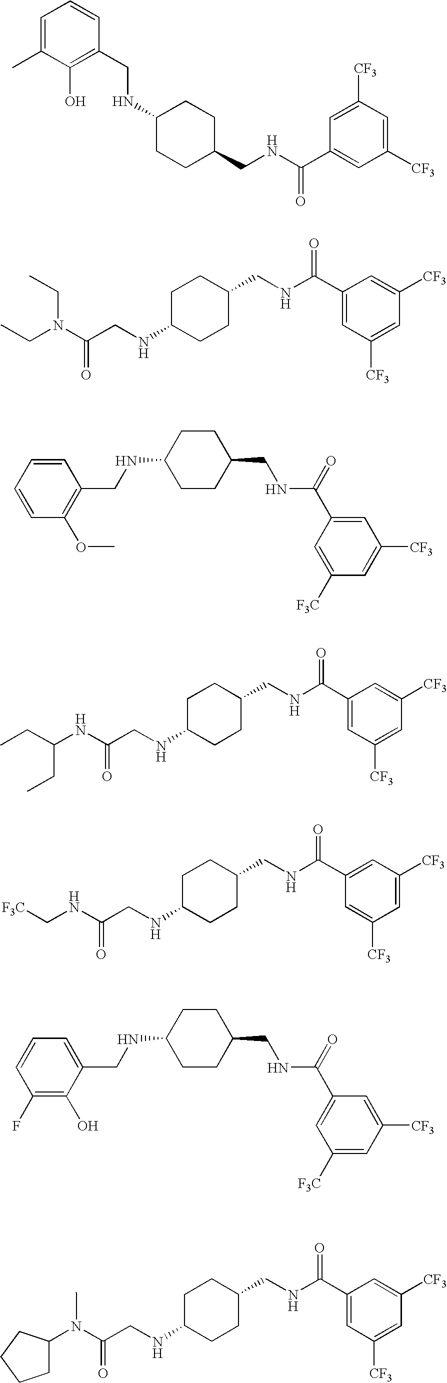 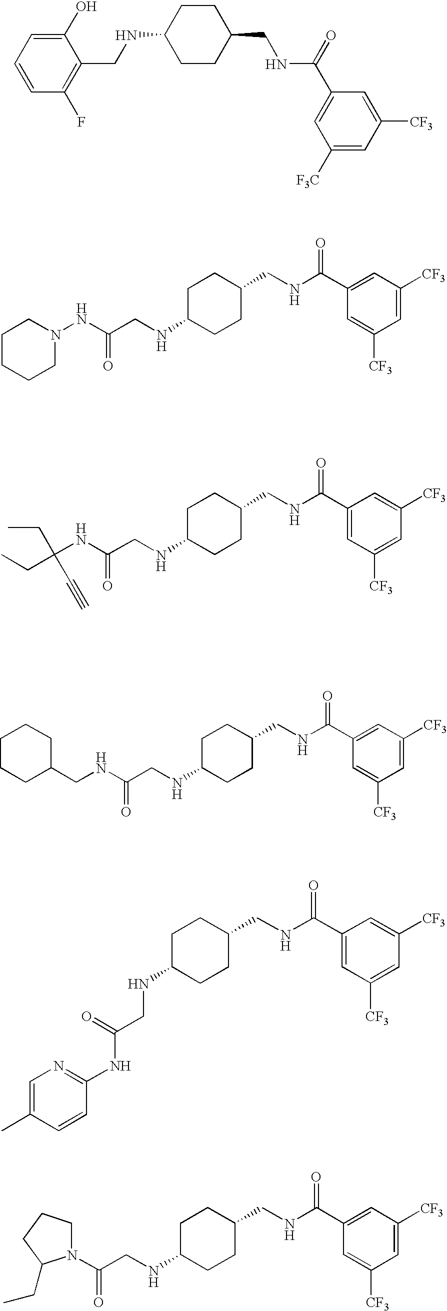 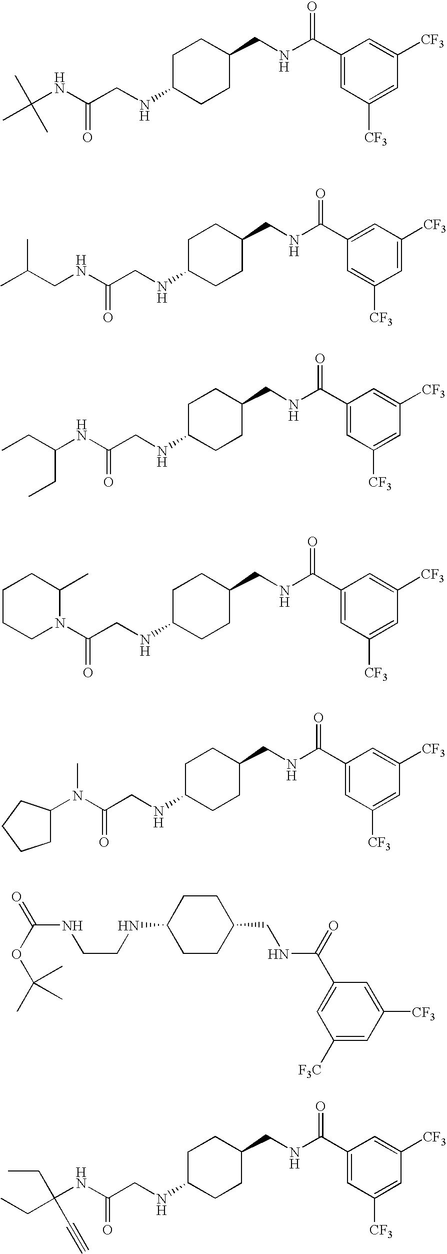 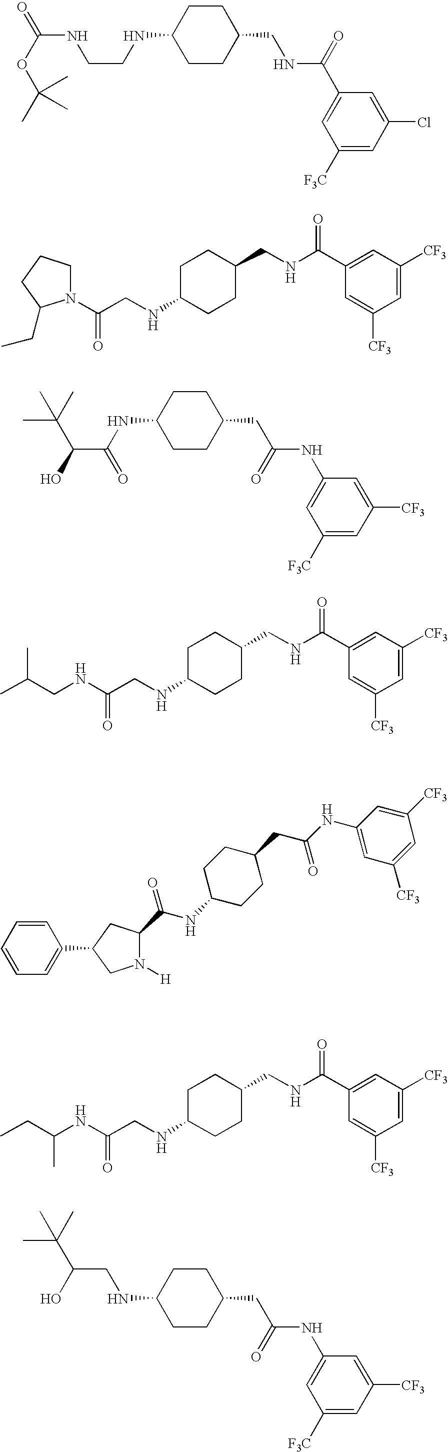 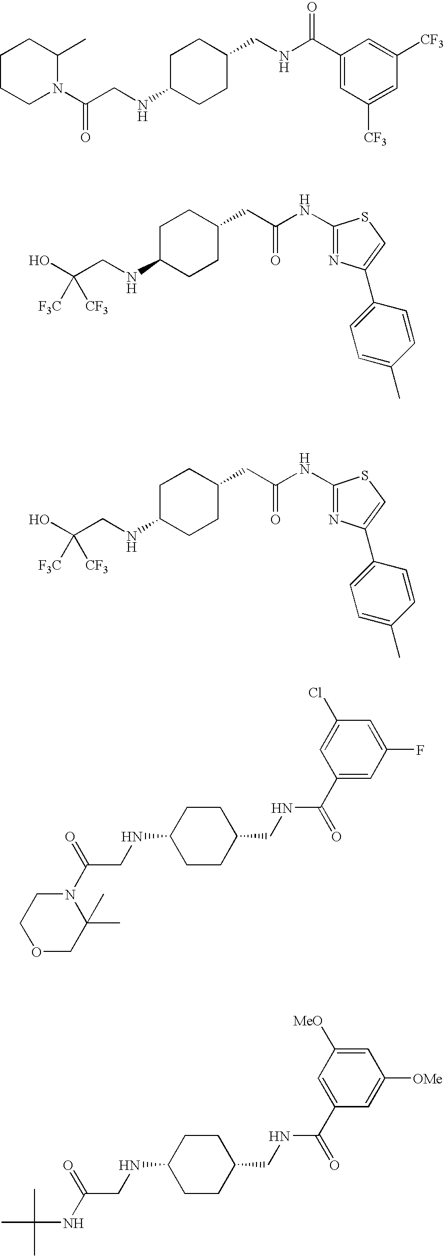 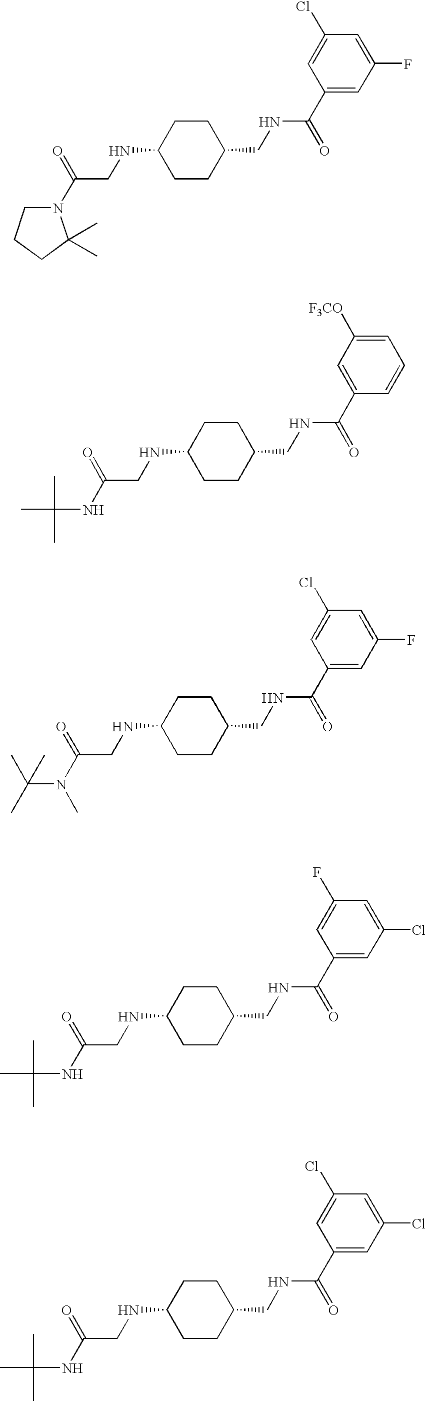 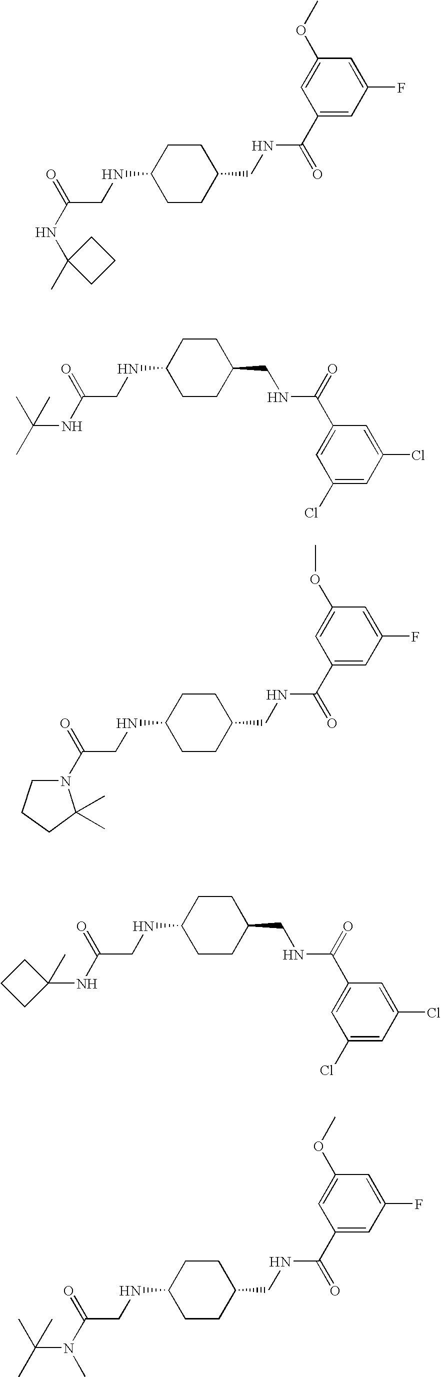 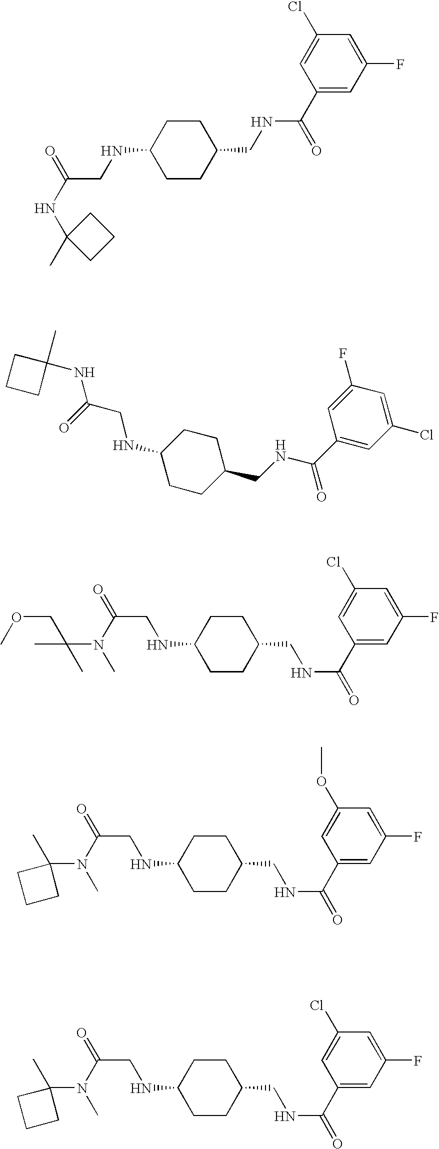
or a pharmaceutically acceptable salt of one of these.
